(19) United States
(12) Patent Application Publication
(10) Pub. No.: US 2017/0056524 A1
(43) Pub. Date: Mar. 2, 2017

(54) COMPOSITIONS AND METHODS FOR TARGETED EPIGENETIC MODIFICATION

(71) Applicant: THE TRUSTEES OF THE UNIVERSITY OF PENNSYLVANIA, PHILADELPHIA, PA (US)

(72) Inventor: KLAUS H. KAESTNER, Swarthmore, PA (US)

(21) Appl. No.: 15/261,663

(22) Filed: Sep. 9, 2016

Related U.S. Application Data
(63) Continuation of application No. PCT/US2015/020405, filed on Mar. 13, 2015.
(60) Provisional application No. 61/952,317, filed on Mar. 13, 2014.
(60) Provisional application No. 62/110,216, filed on Jan. 30, 2015.

(51) Int. Cl. A61K 48/00; A61K 45/06

(52) U.S. Cl. CPC A61K 48/00; A61K 45/06

(57) ABSTRACT

The present disclosure is directed to compositions and methods for targeting and modulating the epigenetic “state” (e.g., methylation state) of one or more genes. For example, the present disclosure is directed, in part, to transcription activator-like effector (TALE) fusion protein compositions and methods of their use in targeting and modulating the epigenetic state of one or more genes.

### CROSS-REFERENCE TO RELATED APPLICATIONS

The application is a continuation of International Patent Application No. PCT/US2015/020405, filed Mar. 13, 2015, which claims priority to U.S. Provisional Application Ser. No. 61/952,317, filed Mar. 13, 2014, and U.S. Provisional Application Ser. No. 62/110,216, filed Jan. 30, 2015, each of which are incorporated by reference in their entirety.

### STATEMENT REGARDING FEDERALLY-SPONSORED RESEARCH

This invention is made with government support under DK089529 and DK088383 awarded by the National Institute of Health. The government has certain rights in the invention.

### SEQUENCE LISTING

The instant application contains a Sequence Listing which has been submitted electronically in Text format and is hereby incorporated by reference in its entirety. Said ASCII copy, created on Sep. 9, 2016, is named 081406_0264SEQLIST.txt and is 30,293 bytes in size.

### BACKGROUND

Diabetes mellitus is a lifelong chronic disease currently affecting more than 336 million people worldwide, with healthcare costs relating to diabetes and its complications of up to $612 million per day in the U.S. alone. The incidence of diabetes mellitus is increasing in other parts of the world, and it is considered a worldwide epidemic. Diabetes is associated with a variety of physiologic disorders such as obesity, hypertension, dyslipidemia and cardiovascular disease. In addition, diabetes can cause long term microvascular and macrovascular complications, such as nephropathy, neuropathy, retinopathy and atherosclerosis. The high mortality rate and debilitating neuropathies associated with diabetes underline the importance of active medical intervention.

There are two main forms of diabetes. Type 1 (insulin-dependent diabetes mellitus), the second most common chronic disease in children, is due primarily to autoimmune-mediated destruction of pancreatic β-cells, resulting in absolute insulin deficiency. In contrast, Type 2 diabetes (non-insulin dependent diabetes mellitus) is characterized by insulin-resistance and inadequate insulin secretion by β-cells. A significant fraction of individuals originally diagnosed with Type 2 diabetes evolve with time to a Type 1 state. Common to both types of diabetes is the failure of pancreatic islet β-cells, which is defined as a loss of both function, i.e., glucose stimulated insulin secretion, and inadequate β-cell mass, either by increased apoptosis or a failure to proliferate in response to metabolic demand.

The islets of Langerhans, miniature endocrine organs within the pancreas, are essential regulators of blood glucose homeostasis and play a key role in the pathogenesis of diabetes. While for decades insulin deficiency was considered the sole issue relating to diabetes, recent studies emphasize excess glucagon as an important part of diabetes etiology, making diabetes a ‘bihormonal disease’.

There are currently several methods aimed at treating or managing the symptoms of diabetes. The first is lifestyle adjustments aimed at improving endogenous insulin production. This can be achieved by increasing physical activity and bodyweight reduction with diet and behavioral modifications. Unfortunately, most people with Type 2 diabetes never receive sufficient nutritional education or are not capable of complying with a strict diet regimen. Other therapeutic methods involve the use of pharmacological agents to manage the disease.

These pharmaceutical drugs fall into three categories: pancreatic stimulators, insulin sensitizers and exogenously supplied insulin or insulin analogs. Pancreatic stimulators are used to stimulate the pancreas to increase endogenous secretion of insulin. This stimulation of insulin release has been shown to be detrimental during long term use. In particular, it can lead to exhaustion of the pancreatic islets, more specifically, exhaustion of β-cells. Insulin sensitizers, which are commonly used to treat Type II diabetes, are used to improve the cell's sensitivity to the presence of insulin, thereby improving the uptake of glucose into the cells, leading to better blood sugar control. The third group, insulin or insulin analogs, are exogenously supplied to patients suffering from both Type I and Type II diabetes. The most frequently encountered adverse effect of insulin and insulin analogs is hypoglycemia, i.e., low blood sugar.

As diabetes is a chronic, long-duration disease, the pharmacological drugs currently available are taken on a long term basis, which results in severe side effects and can contribute to the complications of the disease. Additionally, there are currently no treatments that target β-cell expansion and regeneration. Accordingly, there exists a need for diabetes treatments that do not result in such adverse effects, and for treatments that can prevent or minimize β-cell failure.

### SUMMARY

The present disclosure is directed, in part, to targeting and modulating the epigenetic “state” (e.g., methylation state) of one or more genes. For example, but not by way of limitation, certain embodiments of the present disclosure are based, in part, on the discovery that pancreatic islet cells display cell-type specific epigenomic plasticity, and that epigenetic manipulation of α-cells allows for cell reprogramming of these cells into functional β-cells. Reprogramming of α-cells into functional β-cells represents a cell-replacement-based therapy for diabetes.

In certain embodiments, the presently disclosed subject matter provides methods of treating or preventing diabetes in a subject, including administering to the subject a therapeutically effective amount of a pharmaceutical composition including an epigenetic modifier that is capable of reprogramming α-cells cells into functional β-cells. In one embodiment, an α-cell from the subject can be treated with the epigenetic modifier in vitro to reprogram the cell to a functional β-cell, and the reprogrammed cell can be reintroduced into the subject, to thereby treat diabetes. In another embodiment, the presently disclosed subject matter provides methods of treating or preventing diabetes in a subject, including administering to the subject a therapeutically effective amount of a pharmaceutical composition including an epigenetic modifier that is capable of reversing the quiescent phenotype of aged β-cells, resulting in increased insulin production and secretion.

In certain embodiments, the epigenetic modifier is administered in conjunction with one or more additional agents for the treatment or prevention of diabetes.

The epigenetic modifier can be, for example, a small molecule, protein, or a nucleic acid molecule, including, for example, a histone methylation inhibitor (such as a H3K27me3-specific inhibitor).

An epigenetic modifier nucleic acid molecule can be expressed by a therapeutic vector. In certain embodiments, the nucleic acid molecule encodes a TALE fusion protein, which includes one or more coding sequences operably linked to a promoter sequence, where the one or more coding sequences encode at least a first polypeptide domain comprising a TALE (Transcription activator like) effector DNA binding domain and at least a second polypeptide domain having epigenetic modifying activity.

In certain embodiments, the first polypeptide domain is specifically directed towards binding to one or more nucleic acid sequences in a target gene that are involved in the control of gene expression. In certain embodiments, the target gene is selected from the group consisting of: Pdx1, Pax4, Arx, Dpp4, Ptprd, and MafA.

In certain embodiments, the second polypeptide domain encodes a catalytic domain of a histone-modifying protein. In certain embodiments, the second polypeptide domain encodes a catalytic domain selected from the group consisting of: a histone methyltransferase; a histone demethylase; a histone acetyltransferase; a histone deacetylase; a nucleic acid methyltransferase; and a nucleic acid demethylase. For example, the second domain can be a domain that is capable of methylating a lysine residue located at position 27 in the tail region of histone H3 (H3K27me3).

Compositions including a vector encoding a nucleic acid molecule encoding the TALE fusion protein are also included in the presently disclosed subject matter.

In certain embodiments, the TALE fusion is a TALE-DMNT fusion.

In certain embodiments, the TALE fusion will comprise a TALE domain that has been modified such that the fusion is compatible with a lentiviral vector. In certain embodiments, the lentiviral vector compatible TALE domain is the result of modifying the TALE nucleic acid sequence to reduce the number of tandem repeats by utilizing the degenercy of the genentic code. In certain embodiments, the lentiviral vector compatible TALE domain is the result of modifying the TALE nucleic acid sequence to reduce the number of tandem repeats by deleting repeates, e.g., reducing the number of repeats from 24 to 18. In certain embodiments, the lentiviral vector compatible TALE domain is the result of modifying the TALE nucleic acid sequence to reduce the number of tandem repeats by utilizing the degenercy of the genentic code and by reducing the number of repeats via deletion.

In certain embodiments the TALE fusion can increase cell replication by targeting a cell cycle regulatory gene. In certain embodiments, the cell cycle regulartory gene is p16 (CDKN2A) locus

### BRIEF DESCRIPTION OF THE FIGURES

FIG. 1A-1D. Study design for determination of the transcriptome and differential histone marks in sorted human islet cells. (FIG. 1A) Human islets were dispersed and subjected to FACS to obtain cell populations highly enriched for α-, β-, and exocrine (duct and acinar) cells. Chromatin was prepared and precipitated with antibodies for H3K4me3 and H3K27me3 followed by high-throughput sequencing (ChIP-Seq) (H3K4me3: n=4α, n=4β, n=2exo, H3K27me3: n=3α, n=3β, n=2exo). RNA-Seq analysis was performed to determine mRNA and mRNA levels (n=3α, n=3β, n=2exo). (FIG. 1B) Sample purity assessment. Normalized insulin and glucagon expression levels of the individual α- and β-cell populations were obtained by qRT-PCR to calculate the contamination by the opposite cell population, revealing high sample purity (2.5-10.3% contamination in the α- and 2-13.1% contamination in the β-cell populations; details in supplemental methods). (FIG. 1C) Analysis pipeline for H3K4me3 and H3K27me3 ChIP-Seq data. Peak-calling (H3K4me3: GLITR; H3K27me3: STAR) on individual replicates, followed by signal-pooling, was employed to assess histone modification profiles of α-, β-, and exocrine cells. Heatmap analysis confirmed reproducibility of replicates. (FIG. 1D) Genome browser image of the PDX1 locus showing H3K4me3 enrichment in α-, β-, and exocrine cells, and H3K27me3 enrichment only in α-cells (defined as monovalent H3K4me3 enrichment in β- and exocrine cells, bivalent mark in α-cells; CpG islands: bars).

FIG. 2A-2B. Genome-wide transcriptome analysis using RNA-Seq confirms high purity of sorted cell populations and reveals cell-type specific gene expression. (FIG. 2A) Principal component analysis displays distinct cell populations and clustering of replicates (n=3α, n=3β, n=2exocrine), which confirms the high purity of the sorted cell populations (dots: replicates, crosses: averages). (FIG. 2B) Heatmap analysis shows groups of genes with distinct expression patterns across cell types (columns: cell types, rows: genes). The top, middle, and bottom bars on the left side of the heatmap indicate α-, β-, and exocrine specific gene clusters, respectively. The darker portion of these bars indicate stronger cell-type specificity of the gene cluster. Important genes are highlighted, including genes found to be associated with diabetes in genome-wide association studies (marked with asterisks). The complete gene lists of α-, β-, and exocrine-specific genes are provided in Branswig et al. J. Clin. Invest. 2013 123(3):1275-84.

FIG. 3A-3G. Human α-, β-, and exocrine cells exhibit convergent monovalent H3K4me3 and H3K27me3 profiles, which correlate highly with genome-wide expression data. (FIG. 3A) The majority of H3K4me3-marked genes are shared between α-, β-, and exocrine cells (their overlap is indicated in the lower portion of the bars, 83-95%). (FIG. 3B) H3K27me3-modification patterns are similar among pancreatic cell types (73-83%, lower portion of the bars). (FIG. 3C and FIG. 3D) Heatmap analysis (columns: individual samples, rows: genes) confirms low inter-individual variability for all H3K4me3 (FIG. 3C) and H3K27me3 (FIG. 3D) peaks identified from the pooled data (peaks called by algorithms are indicated by the solid bars on the left of the heatmaps). All pairs of columns in every heatmap are significantly correlated with p-value <2.2E-16 (cor.test function in R). (FIG. 3E, FIG. 3F, and FIG. 3G) Expression values obtained by RNA-Seq for genes grouped by their histone modification status in each cell type are shifted significantly above or below 0 on the log 2 scale (corresponding to RPKM=1 on the non-log scale; R ‘wilcox.test’ function, asterisks indicate p<2.2-16). A shift above this value indicates highly expressed genes and was observed for gene groups marked solely by H3K4me3 in all cell types. A shift below this value indicates low or non-expressed genes and was observed in all bivalently marked gene groups and monovalently H3K27me3-marked genes in all cell types. Therefore, the histone modification states are significantly correlated with gene expression levels.

FIG. 4A-4D. Human α-cells demonstrate a higher number of bivalently marked genes than β- and exocrine cells. (FIG. 4A) α-cells display more bivalently marked loci than β- and exocrine cells. Nearly half of the genes bivalently marked in α-cells carry a monovalent mark in β-cells (top two portions of the left bar corresponding to H3K4me3 and H3K27me3 marks in β-cells, respectively). (FIG. 4B) 406 genes are marked bivalently in β-cells, but monovalently by H3K4me3 in α-cells, and gene ontology analysis for these genes shows three modestly enriched categories: regulation of RNA metabolic process, regulation of transcription, and transcription. (FIG. 4C) Genes marked bivalently in α-, but monovalently by H3K27me3 in β-cells are significantly enriched for developmental processes. For detailed GO analysis see table 3. (FIG. 4D) Comparison of transcriptional regulators marked bivalently in hESC (22) to the histone modification signatures of human α- and β-cells reveals a higher overlap between α-cells and hESC (44%, right pie chart) than between β-cells and hESC (26%, left pie chart). Many of the 31 genes marked bivalently both in α-cells and hESC carry the repressive mark in β-cells (43%, darkest portion of inset).

FIG. 5A-5C. Human α-cells display higher bivalency in genes encoding for β-cell transcriptional regulatory proteins. (FIG. 5A) α-cells display a higher degree of bivalency for genes important in regulation of transcription than for genes implicated in ion transport. β-cell signature genes obtained from RNA-Seq analysis of sorted α-, β-, and exocrine cells in FIG. 2B (marked by dark portion of middle box in FIG. 2B) were grouped by gene ontology analysis into those functioning in ion transport or regulation of transcription. Next, their epigenetic status was analyzed separately for α-, β-, and exocrine specific genes. Of the β-cell enriched ion transport genes, only 6% and 15% were marked bivalently in exocrine and β-cells, respectively, while 29% carried this mark in α-cells. For β-cell signature genes involved in transcriptional regulation, 42% were marked as bivalent in α-cells, but only 16% and 13% in β-cells and exocrine cells, respectively. (FIG. 5B and FIG. 5C) Schematic representation of the histone modification status of a relevant subset of human α- and β-cell signature genes, as determined by RNA-Seq analysis (FIG. 2B), known to be important for endocrine cell function. The histone modification status of α-cells is depicted above, the histone modification status of β-cells is shown below each gene of interest. (FIG. 5B) The α-cell expressed genes HNF1A, PCSK2, IRX2, GCG, and DPP4 are marked monovalently by H3K4me3 in α-cells. Many α-specific gene loci carry a monovalent H3K27me3 mark in β-cells, however, the two GWAS loci DPP4 and PTPRD are marked bivalently in β-cells. Interestingly, the loci of 32 IRX1 and the α-cell specific transcription factor ARX are marked bivalently in α-cells, but monovalently by H3K27me3 in β-cells. (FIG. 5C) Within this subgroup of genes, β-cell expressed genes are marked monovalently by H3K4me3 in β-cells, with the exception of HDAC9 which is marked bivalently. Remarkably, many β-cell expressed genes are marked bivalently in α-cells, including the crucial insulin-synthesis enzyme PCSK1, the GLP1-receptor (GLP1R), and two essential β-cell specific transcription factors MAFA and PDX1.

FIG. 6A-6F. Inhibition of histone methyltransferases leads to partial endocrine cell-fate conversion. (FIG. 6A) H3K27me3 ChIP-Seq analysis of human islets shows decreased H3K27me3 levels at the ARX, MAFA and PDX1 loci following treatment of human islets with the histone methyltransferase inhibitor Adox. (FIG. 6B) Adox-treatment of human islets results in co-localization of glucagon and insulin granules within the same cell (arrow) indicating partial endocrine-cell fate conversion, which was not seen in vehicle-treated islets (control). Original magnification 63×. (FIG. 6C) Treatment of human islets with Adox results in co-localization of the β-cell specific transcription factor Pdx1 and glucagon, further indicating endocrine reprogramming (white arrows: glucagon-positive, Pdx1-negative cells; gray arrows: glucagon-positive, Pdx1-positive cells). The images on the right correspond to the area within box. Original magnification 63×. (FIG. 6D) Quantification of glucagon-positive, Pdx1-positive cells in untreated and Adox treated human islets reveals many double-positive cells after Adox-treatment, indicating 33 initiation of reprogramming events in α-cells. (FIG. 6E) Adox-treatment of human islets leads to a decrease in NKX6-1 and MAFA levels in β-cells (n=3α, n=3β, n=2 treated α, n=2 treated β), an increase in PDX1-levels, and no change in INS, GCG, and PDX1 levels. (FIG. 6F) In Adox-treated α-cells we observe no change in INS and GCG expression, a slight decrease in NKX6-1 and MAFA levels, and an increase of ARX and PDX1 expression.

FIG. 7: Separate analysis of bivalent marks in one donor (CITH068) confirms the higher number of bivalent marks in α-cells.

FIG. 8A-8B: Integrative analysis of α- and β-cell signature genes using the ChIP-Seq data sets. (FIG. 8A) Quantitative analysis of H3K4me3 and H3K27me3 levels in the strongly cell-type specific genes (α-strong (left), β-strong (middle), exo-strong (left), as bars in FIG. 2B). As expected, signature genes show increased H3K4me3 levels in their respective cell type. H3K4me3 enrichment of alpha specific genes were found at comparable levels in α- and β-cells, but increased levels in α-cells and decreased levels in β-cells were found in β-cell specific genes. This indicates higher prevalence of H3K4me3 levels in α-cells, repressing β-cell signature genes, and not vice versa. (FIG. 8B) Analysis of the histone modification landscape in α-cell signature genes revealed comparable percentage of bivalently marked genes across α-, β-, and exocrine cells. Of the α-cell enriched ion transport genes, 21%, 28% and 31% were marked bivalently in α-, β-, and exocrine cells. For the α-cell signature genes involved in transcriptional regulation, 17%, 14% and 21% were marked as bivalent in α-, β-, and exocrine cells, respectively.

FIG. 9A-9D: Treatment of human islets with Adox. (FIG. 9A) Treatment of human islets with histone methyltransferase inhibitor Adox leads to decrease in H3K4me3-enrichment. (FIG. 9B) Treatment of human islets with histone methyltransferase inhibitor Adox leads to co-localization of glucagon and insulin granules in pancreatic cells (arrows). Original magnification 63×. (FIG. 9D) Adox-treatment of islets from GlucagonCre; Rosa26EYFP mice results in the occurrence of insulin granules in YFP+ cells (box), indicating partial α- to β-cell fate conversion, which was not observed in control islets (FIG. 9C). Original magnification 60×.

FIG. 10. Base resolution CpG methylation status of the p16 (Cdkn2a) locus in old (24 months, top) and young (6 weeks, middle) β-cells. The height of the bars indicates the percent methylation each CpG. Note that the central CpGs are demethylated as the mice age, indicating activation of the p16 locus, correlating with decreased proliferation and increased p16 expression.

FIG. 11A-11B. Schema for TALE-mediated epigenetic targeting. (FIG. 11A) A hypothetical cell cycle gene is bivalently marked in old, but monovalently marked by H3K4me3 in young β-cells. The catalytic domain of the JMJD3-histone demethylase is targeted specifically to the promoter of this gene using TALE repeats (indicated as boxes in FIG. 11B). Removal of the H3K27me3 repressive mark allows for reactivation of the gene, and promotion of replicative ability in aged j-cells. The lentiviral vector for the expression of the TALE-JMJD3 effector encodes a bicistronic message, allowing for expression of eGFP using the viral 2A sequence.

FIG. 12. Single-cell assay of glucose responsiveness of human I-cells. Left panel: bright-field image of single islet cells captured on cover slip. Middle panel: Fura2 fluorescence, indicative of intracellular [Ca] at high glucose. Up to 50 cells can be recorded simultaneously. Right panel: Cytosolic calcium of cell number 8 from middle panel. Note the rapid response to elevated glucose levels.

FIG. 13. Strategy for construction of GlucagonCreER BAC transgene.

FIG. 14A-14B. Schema for TALE-mediated epigenetic targeting. (FIG. 14A) A β-cell regulator such as Pdx1 or MafA is bivalently marked in α-cells, but monovalently marked by H3K4me3 in β-cells. The catalytic domain of the JMJD3-histone demethylase is targeted specifically to the promoter of either or both genes using TAL repeats (indicated as boxes in FIG. 14B). Removal of the H3K27me3 repressive marks allows for reactivation of the gene in α-cells, and promotion of reprogramming towards β-cells. The lentiviral vector for the expression of the TAL-JMJD3 effector encodes a bicistronic message, allowing for expression of eGFP using the viral 2A sequence.

FIG. 15A-15C. Targeted CpG methylation of the p16 (CDKN2A) locus using TALE-DNMT fusion proteins. (FIG. 15A) TALE-DNMT strategy for altering the epigenetic state of the p16 (CDKN2A) promoter. Locus-specific TALEs were fused to the catalytic domain of DNA methyltransferase (p16 TALE-DNMT), or a catalytically inactive DNA methyltransferase with the point mutation E752A (p16 TALE-DNMT Mut). (FIG. 15B) Detailed diagram of TALE-DNMT construct and target site in the p16 (CDKN2A) locus. Black boxes indicate the three exons of the p16 transcript, and green boxes indicate CpG islands. The TALE-DNMT was targeted to the CpG island at the promoter just before the transcription start site. Legend on the right side of the diagram indicates which nucleotide is targeted by each of the four different TALE repeat monomers, which are color-coded. (FIG. 15C) Percent methylation of individual CpGs within the CDKN2A promoter in FACS-sorted GFP-positive populations compared to untreated HeLa cells. HeLa cells were transfected with the p16 TALE-DNMT wild-type or p16 TALE-DNMT mutant construct and cultured for 48 hours. Cells were then FACS sorted for GFP to isolate transfected populations. DNA methylation was quantified by sequencing of PCR-amplified bisulfite-converted genomic DNA. Graphs reflect percent DNA methylation at each CpG and its position relative to the transcription start site (mean±SEM; n=3). Diagram below the graph illustrates the region of the p16 (CDKN2A) promoter that was analyzed. Data points outlined in black are significantly elevated in the p16 TALE-DNMT population compared to the p16 TALE-DNMT mutant population (P<0.05).

FIG. 16A-16C. Minimizing direct repeats permits lentiviral expression of TALE fusion proteins. HeLa cells were infected with p16 jumbled TALE-DNMT, p16 jumbled TALE-DNMT mutant, or GFP control lentiviruses and harvested after four days. (FIG. 16A) Western blot of HeLa cells infected with p16 jTALE-DNMT or p16 jTALE-DNMT mutant lentivirus showing production of the fulllength protein. (FIG. 16B) PCR amplification of the full-length TALE repeat moiety from genomic DNA (gDNA), demonstrating integration of the intact construct into the host genome, and from cDNA, demonstrating transcription of full length mRNA, in infected HeLa cells. jTALE, jumbled TALE; WPRE, Woodchuck hepatitis virus posttranscriptional regulatory element. (FIG. 16C) Percent DNA methylation of the p16 (CDKN2A) locus in HeLa cells infected with p16 jTALE-DNMT wild-type and p16 jTALE-DNMT mutant lentivirus (mean±SEM; n=3). Data points outlined in black indicate CpGs in which DNA methylation is significantly elevated in p16 jTALE-DNMT wild-type infected cells compared to p16 jTALE-DNMT mutant infected cells (P<0.05).

FIG. 17A-17E. Targeted CpG methylation at the p16 (CDKN2A) locus results in decreased gene expression in primary human cells. Primary human fibroblasts were transduced with p16 jTALE-DNMT wild-type or p16 jTALE-DNMT mutant lentiviruses and incubated for four days. (FIG. 17A) Percent DNA methylation of CpGs within the p16 (CDKN2A) promoter region. Graphs reflect percent DNA methylation at each CpG (mean±SEM; n=3) and position relative to the transcription start site. Data points outlined in black are significantly elevated in the p16 jTALE-DNMT population compared to the p16 jTALE-DNMT mutant population (P<0.05). (FIG. 17B) p16 transcript expression in fibroblasts treated with p16 jTALE-DNMT wild-type or mutant lentiviruses relative to the mutant negative control. Expression levels were normalized to HPRT1 mRNA levels (mean±SEM; n=3) *, P<0.05. (FIG. 17C) Average percent DNA methylation of CpGs at each CpG island within the p16 (CDKN2A) locus and at β-actin (ACTB), a housekeeping gene located on a different chromosome. The diagram below the graph illustrates the position of CpG islands at the p16 (CDKN2A) locus. (mean±SEM; n=3) *, P<0.05; **, P<0.01. (FIG. 17D) mRNA expression of genes adjacent to p16 (CDKN2A) in lentivirally transduced human fibroblasts, determined as described in (FIG. 17B). The diagram above the graph indicates the position of each gene relative to p16 (CDKN2A). (FIG. 17E) Average percent methylation at genes adjacent to p16 (CDKN2A).

FIG. 18A-18E. Alterations in p16 levels due to p16 TALE-DNMT results in increased proliferation in primary human cells. (FIG. 18A) EdU incorporation of human fibroblasts infected with wild-type or mutant p16 jTALE-DNMT lentivirus. Fibroblasts were plated in chamber slides and infected for 72 hours. Cells were then incubated with EdU for 1 hour and stained for EdU incorporated into newly replicated DNA by immunofluorescence. Cell nuclei are stained blue (DAPI) and EdU positive nuclei are stained red. (FIG. 18B) Percent EdU incorporation of cells infected with p16 jTALE-DNMT wild-type or mutant lentivirus, with or without co-infection of CMV-p16 lentivirus. Percent EdU incorporation was calculated as the number of EdU positive cells divided by the total number of cells. Three random images were counted for each biological replicate (mean±SEM; n>4). ***, P<0.001; n.s., not significant. (FIG. 18C) Population doubling time of human fibroblasts infected with p16 jTALE-DNMT wild-type or mutant lentivirus. Initial cell number was determined prior to plating cells, and final cell number 4 days post-infection (mean±SEM; n=4). Population doubling time (DT) in days was calculated as DT=T ln 2/ln(Xf/Xi). *, P<0.05; T, incubation time (days); Xi, initial cell number; Xf, final cell number. (FIG. 18D) Transcript levels of cell cycle regulators in human fibroblasts transduced with wild-type or mutant p16 jTALE-DNMT lentivirus. Total RNA was extracted 4 days post-infection and mRNA levels determined by qRT-PCR as described in the Methods section. mRNA levels are expressed as relative to mutant p16 jTALE-DNMT transduced cells, which was set to 1 (mean±SEM; n=3). (FIG. 18E) Average percent DNA methylation of CpGs at the nearest CpG island of cell cycle regulators evaluated in (FIG. 18D). Average DNA methylation was measured by PCR amplification of bisulfite converted genomic DNA followed by high-throughput sequencing (mean±SEM; n=3). **, P<0.01; ***, P<0.001.

FIG. 19. PCR primers for amplification of bisulfite converted genomic DNA. Primers were designed to PCR amplify regions of interest from bisulfite converted genomic DNA. Each primer pair amplifies an approximately 250-300 base pair region within the CpG island closest to the transcription start site of the gene analyzed. In instances where there was not a CpG island near the gene, the promoter was evaluated. Amplicons were subsequently used to prepare DNA sequencing libraries for DNA methylation analysis.

FIG. 20. qPCR primers for gene expression analysis.

FIG. 21. Coding sequence of p16 TALE-DNMT3α-3L.

FIG. 22. Jumbled p16 TALE repeat domain. Degeneracy of the genetic code was used to minimize direct nucleotide repeats in the TALE repeat domain without affecting the coding sequence.

FIG. 23A-23B. p16 TALE-DNMT strategy can be employed in primary human coronary artery smooth muscle cells to decrease p16 expression. Primary human coronary artery smooth muscle cells were infected with p16 jTALEDNMT wild-type or mutant lentivirus. After 4 days of infection, cells were harvested and assessed for DNA methylation (FIG. 23A) and p16 (CDKN2A) expression (FIG. 23B) as described in the Example 6.

FIG. 24A-24B. Multiple p16 TALE-DNMT constructs can be designed to decrease p16 expression. An additional TALE-DNMT (p16 jTALE-DNMT.2) was designed to target the p16 (CDKN2A) promoter region 118 to 139 base pairs upstream of the transcription start site. Wild-type and mutant infected with p16jTALE-DNMT.2 were evaluated for DNA methylation (FIG. 24A) and p16 (CDKN2A) gene expression (FIG. 24B) as described in prior human fibroblast experiments.

### DETAILED DESCRIPTION

### 1. Introduction

The present disclosure is directed, in part, to targeting and modulating the epigenetic “state” (e.g., methylation state) of one or more genes. For example, but not by way of limitation, certain embodiments of the present disclosure are based, at least in part, on the discovery that pancreatic islet cells display cell-type specific epigenomic plasticity, and that epigenomic manipulation of α-cells allows for cell reprogramming of these cells into functional β-cells.

In particular, the present inventors have performed epigenomic profiling of human cell populations highly enriched for α, β, and exocrine (duct and acinar) cells and have determined their H3K4me3 and H3K27me3 histone modification patterns genome-wide. They have found an α-cell specific histone modification pattern that preserves α-cells in a plastic epigenomic state, with numerous genes (approximately 3,000) bivalently marked by the activating H3K4me2 histone modification and the repressing H3K27me3 histone modification. Nearly half (approximately 1,400) of these genes are resolved to a monovalent state in β-cells, which thus exhibit a more fixed epigenetic state. Moreover, treatment of pancreatic islets with a histone methyltransferase inhibitor caused co-expression of both glucagon and insulin in humans, and partial α- to β-cell fate conversion in mice. Therefore, the inventors discovered that the identified epigenetic plasticity of α-cells can be exploited by targeted reprogramming of α-cells into functional β-cells.

The use of epigenetic manipulation to reprogram α-cells into functional β-cells is a cell-replacement-based therapy for diabetes. In particular, in Type II diabetes, conversion of α-cells into functional β-cells results in decreased glucagon and increased insulin production. With respect to Type I diabetes, donor eyelet cells are often transplanted into a patient, along with immunosuppressants. Due to a low number of organ donors, additional sources of cells are needed. In certain embodiments, the present disclosure provides for transplanting reprogrammed eyelet cells into a patient, which therefore results in an increased number of transplanted β-cells without reliance on donors.

Accordingly, in certain embodiments, the present disclosure provides compositions and methods for treating or preventing diabetes in a subject by administering a therapeutically effective amount of an epigenetic modulator that is capable of reprograming α-cells into functional β-cells, to the subject, e.g., a mammal. The present disclosure also provides methods for treating α-cells cells ex vivo with an epigenetic modulator capable of reprograming α-cells into functional β-cells, and reintroducing the treated cells into a subject to treat or prevent diabetes.

In addition, in certain embodiments, the present disclosure also provides methods and compositions for the removal of repressive DNA methylation or histone marks in β-cells using an epigenetic modifier, resulting in reversal of the quiescent phenotype of aged β-cells to restore proliferative potential to mature β-cells, and thereby treat or prevent diabetes in a subject.

In certain embodiments, the epigenetic modifier can be a small molecule or a protein. In certain embodiments, the epigenetic modifier is a histone methylation inhibitor, e.g., an H3K27me3-specific inhibitor.

In certain embodiments, the epigenetic modifier is a locus-specific epigenetic modifier. For example, the epigenetic modifier can target a regulatory gene such as Pdx1, Pax4, Arx, Dpp4, Ptprd, or MafA, or one or more additional regulatory genes that have been shown to be bivalently marked in α-cells, but monovalently marked in β-cells. The nucleotide and amino acid sequences of these target regulatory genes are well known in the art.

DNA methylation, demethylation, histone demethylation, acetylation, and histone acetylation can be targeted, for example, using a TAL (Transcription activator-like) effector fusion protein engineered to target specific promoters and enhancers of a target regulatory gene.

In addition to the diabetes-relarted aspects described herein, in certain embodiments, the present disclosure is directed compositions and methods relating to modulating the epigenetic state (e.g., methylation state) of one or more genes that are not necessarily related to diabetes or pancreatic cell reprogramming. For example, but not by way of limitation, the present disclosure relates to compositions and methods where the TALE fusion is a TALE-DMNT fusion.

In certain embodiments, the TALE fusion will comprise a TALE domain that has been modified such that the fusion is compatible with a lentiviral vector. In certain embodiments, the lentiviral vector compatible TALE domain is the result of modifying the TALE nucleic acid sequence to reduce the number of tandem repeats by utilizing the degenercy of the genentic code. In certain embodiments, the lentiviral vector compatible TALE domain is the result of modifying the TALE nucleic acid sequence to reduce the number of tandem repeats by deleting repeates, e.g., reducing the number of repeats from 24 to 18. In certain embodiments, the lentiviral vector compatible TALE domain is the result of modifying the TALE nucleic acid sequence to reduce the number of tandem repeats by utilizing the degenercy of the genentic code and by reducing the number of repeats via deletion.

For example, but not by way of limitation, in certain embodiments the TALE fusion is capable of increasing cell replication. In certain embodiments, such increase in cell replication is achieved by targeting a cell cycle regulatory gene. In certain embodiments, the cell cycle regulartory gene is p16 (CDKN2A) locus.

### 2. Definitions

The term “reprogramming” as used herein, refers to the altering or removing of epigenetic modifications from the nucleus of a cell. Reprogramming facilitates a reduction in cell fate commitment and, thus, the differentiation state of the cell as a whole. In essence, reprogramming includes returning a somatic differentiated or committed nucleus to a gene expression, epigenetic, and functional state characteristic of an embryonic, germ, or stem cell, or its conversion into the epigenetic state of a different type of differentiated somatic cell.

The term “epigenetic modification” refers to the chemical marking of the genome by an epigenetic modifier. Epigenetic marks can include DNA methylation (imprints) as well as methylation and acetylation of proteins associated with DNA, such as histones. Parent-of-origin-specific gene expression (either from the maternal or paternal chromosome) is often observed in mammals and is due to epigenetic modifications. In the parental germlines, epigenetic modification can lead to stable gene silencing or activation. Other modifications such as the histone marks lead to a stable or semi-stable expression state of a cell, defining the properties of a differentiated somatic cell.

As used herein, the term “diabetes” is intended to mean all diabetic conditions, including, without limitation, diabetes mellitus, genetic diabetes, type 1 diabetes, type 2 diabetes, and gestational diabetes. The term “diabetes” also refers to the chronic disease characterized by relative or absolute deficiency of insulin that results in glucose intolerance. Type 1 diabetes is also referred to as insulin dependent diabetes mellitus (IDDM) and also includes, for example, juvenile-onset diabetes mellitus. Type 1 is primarily due to the destruction of pancreatic .beta.-cells. Type 2 diabetes mellitus is also known as non-insulin dependent diabetes mellitus (NIDDM) and is characterized, in part, by impaired insulin release following a meal. Insulin resistance can also be a factor leading to the occurrence of type 2 diabetes mellitus. Genetic diabetes is due to mutations which interfere with the function and regulation of .beta.-cells.

Diabetes, as used herein, is characterized as a fasting level of blood glucose greater than or equal to about 130 mg/dl or as a plasma glucose level greater than or equal to about 180 mg/dl as assessed at about 2 hours following the oral administration of a glucose load of about 75 g or following a meal. As understood by the skilled artisan, characteristics used in identifying diabetes are subject to change and the latest standards, such as those disclosed by the World Health Organization, can be used to define diabetes as provided in the present disclosure.

The term “diabetes” is also intended to include those individuals with hyperglycemia, including chronic hyperglycemia, hyperinsulinemia, impaired glucose homeostasis or tolerance, and insulin resistance. Plasma glucose levels in hyperglycemic individuals include, for example, glucose concentrations greater than normal as determined by reliable diagnostic indicators. Such hyperglycemic individuals are at risk or predisposed to developing overt clinical symptoms of diabetes.

An “individual” or “subject” herein is a vertebrate, such as a human. Mammals include, but are not limited to, humans, primates, farm animals, sport animals, rodents and pets.

An “effective amount” of a substance as that term is used herein is that amount sufficient to effect beneficial or desired results, including clinical results, and, as such, an “effective amount” depends upon the context in which it is being applied. In the context of administering a composition to treat or prevent diabetes, an effective amount of an epigenetic modifier is an amount sufficient to treat and/or ameliorate diabetes as well as decrease the severity or prevent a particular diabetes-related complication (i.e., retinopathy, glaucoma, cataracts, heart disease, stroke, hypertension, neuropathy, dermopathy, gum disease, etc.). The decrease can be a 10%, 20%, 30%, 40%, 50%, 60%, 70%, 80%, 90%, 95%, 98% or 99% decrease in severity of complications. An effective amount can be administered in one or more administrations.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of disease, stabilized (i.e., not worsening) state of disease, prevention of disease, delay or slowing of disease progression, and/or amelioration or palliation of the disease state. “Treatment” can also mean decreasing the severity or preventing a particular diabetes-related complication (i.e., retinopathy, glaucoma, cataracts, heart disease, stroke, hypertension, neuropathy, dermopathy, gum disease, etc.). The decrease can be a 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

The term “expression vector” is used to denote a DNA molecule that is either linear or circular, into which another DNA sequence fragment of appropriate size can be integrated. Such DNA fragment(s) can include additional segments that provide for transcription of a gene encoded by the DNA sequence fragment. The additional segments can include and are not limited to: promoters, transcription terminators, enhancers, internal ribosome entry sites, untranslated regions, polyadenylation signals, selectable markers, origins of replication and such like. Expression vectors are often derived from plasmids, cosmids, viral vectors and yeast artificial chromosomes; vectors are often recombinant molecules containing DNA sequences from several sources.

The term “operably linked,” when applied to DNA sequences, for example in an expression vector, indicates that the sequences are arranged so that they function cooperatively in order to achieve their intended purposes, i.e., a promoter sequence allows for initiation of transcription that proceeds through a linked coding sequence as far as the termination signal.

A “nucleic acid molecule” is a single or double stranded covalently-linked sequence of nucleotides in which the 3′ and 5′ ends on each nucleotide are joined by phosphodiester bonds. The polynucleotide can be made up of deoxyribonucleotide bases or ribonucleotide bases. Polynucleotides include DNA and RNA, and can be manufactured synthetically in vitro or isolated from natural sources.

The term “promoter” as used herein denotes a region within a gene to which transcription factors and/or RNA polymerase can bind so as to control expression of an associated coding sequence. Promoters are commonly, but not always, located in the 5′ non-coding regions of genes, upstream of the translation initiation codon. The promoter region of a gene can include one or more consensus sequences that act as recognizable binding sites for sequence specific DNA binding domains of DNA binding proteins. Nevertheless, such binding sites can also be located in regions outside of the promoter, for example in enhancer regions located in introns or downstream of the coding sequence.

A “regulatory gene” is a gene involved in controlling the expression of one or more other genes.

### 3. Epigenetic Modifiers

An epigenetic modifier can include, for example, small molecule, polypeptide, or nucleic acid molecule modifiers.

In particular, certain embodiments, small molecule inhibitors can be useful in the ex vivo application of the methods of the present disclosure, as there is no danger in producing side-effects related to systemic administration. Small molecule histone deacetylase (HDAC) inhibitors are examples of small molecule epigenetic modifiers that can be used in the methods of the present disclosure. Vorinostat (Merck) is an FDA-approved HDAC inhibitor. Romidepsin (Celgene), is another HDAC inhibitor, approved for the treatment of cutaneous T cell lymphoma (CTCL) patients. Two additional HDAC inhibitors (Panobinostat (Novartis) and CI-994 (Pfizer)) are currently being tested in clinical phase III trials for the treatment of cancers. In addition, Chen and Shiaff (Cell Research (2013) 23:326-328; incorporated herein by reference), describe a KDM6 inhibitor that inhibits demethylation mediated by the two related histone H3 lysine 27 demethylases, KDM6A and 6B (UTX and JMJD3). Other small molecule epigenetic modifiers are described in, for example, Piekarz et al. Clin Cancer Res 2009; 15(12) Jun. 15, 2009, incorporated herein by reference.

In certain embodiments, an epigenetic modulator used in the compositions and methods of the present disclosure includes a nucleic acid molecule including first domain encoding a TALE (Transcription activator-like) effector binding domain fused to a second domain encoding the catalytic binding domain of a protein that targets a regulatory gene (e.g., Pdx1, Pax4, Arx, Dpp4, Ptprd, or MafA, or one or more other regulatory genes that are bivalently marked in α-cells and monovalently marked in (3-cells), that is involved in the control of gene expression.

The catalytic binding domain can include, for example, a histone methyltransferase, a histone demethylase, a histone acetyltransferase, a histone deacetylase, a nucleic acid methyltransferase, or a nucleic acid demethylase. In one embodiment, the catalytic binding domain is from a protein capable of methylating a lysine residue located at position 27 in the tail region of histone H3 (H3K27me3) (e.g., histone H3K27me3 demethylase JMJd3).

In certain embodiments, targeted de novo DNA methylation may be accomplished by tethering the catalytic domain of a DNA methyltransferase (DNMT) to DNA binding proteins, e.g., a TALE domain, designed to bind specific gene loci, thereby affecting gene expression

TALE effectors can be designed to uniquely and specifically recognize any 24 bp sequence in the human genome, and can be tethered to the catalytic domains of enzymes, such as transcriptional repressors or nucleases (Boch, 2011; Scholze and Boch, 2011). TALEs are natural type III effector proteins secreted by numerous species of Xanthomonas to modulate gene expression in host plants and to facilitate bacterial colonization and survival (Boch et al., Annu Rev Phytopathol 2010; Bogdanove et al., Curr Opin Plant Biol 2010). Studies have revealed an elegant code linking the repetitive region of TAL effectors with their target DNA-binding site (Boch et al., Science 2009; Moscou et al., Science 2009). Common among the entire family of TAL effectors is a highly conserved and repetitive region within the middle of the protein, consisting of tandem repeats of mostly 33 or 34 amino acid segments. Repeat monomers differ from each other mainly in amino acid positions 12 and 13 (repeat variable di-residues), and computational and functional analyses have revealed a strong correlation between unique pairs of amino acids at positions 12 and 13 and the corresponding nucleotide in the TAL effector-binding site: NI to A, HD to C, NG to T, NN to G (and to a lesser degree A) (Boch et al., Science 2009; Moscou et al., Science 2009; Miller et al., Nat. Biotech 2011; Zhang et al., Nat. Biotech 2011).

In certain embodiments, the TALE fusion protein epigenetic modifier is administered to treat or prevent diabetes, by way of gene therapy. Gene therapy refers to therapy performed by the administration to a subject of an expressed or expressible nucleic acid.

Any of the methods for gene therapy available in the art can be used according to the present disclosure. Exemplary methods are described below. For general reviews of the methods of gene therapy, see Kron and Kreppel, Curr Gene Ther 12(5):362-73 (2012); Yi et a. Curr Gene Ther 11(3):218-28 (2011); Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, N Y (1993); and Kriegler, Gene Transfer and Expression, A Laboratory Manual, Stockton Press, NY (1990).

In certain embodiments, compositions disclosed herein include nucleic acid sequences encoding a TALE fusion protein epigenetic modifier, said nucleic acid sequences being part of expression vectors that express the TALE fusion protein epigenetic modifier or functional fragments thereof in a suitable host. In certain embodiments, such nucleic acid sequences have promoters operably linked to the TALE coding region, said promoter being inducible or constitutive, and, optionally, tissue-specific. Because of their universal activity, viral promoters were components of many first-generation vectors. However, many of the viral promoters, such as the cytomegalovirus (CMV) promoter, are attenuated or completely shut-off in specific organs. In comparison to viral or housekeeping promoters, tissue-specific promoters direct higher levels of expression in vivo. (Atta, World J Gastroenterol. 2010 Aug. 28; 16(32): 4019-4030). Specific promoters to be used for the targeting of pancreatic alpha cells include that of the pre-proglucagon gene (Nian M, Gu J, Irwin D M, Drucker D J. Am. J. Physiol. Regul. Integr. Comp. Physiol. 2002 January; 282(1): R173-83).

Delivery of nucleic acid into a subject or cell, e.g., a pancreatic cell, can be either direct, in which case the subject or cell, e.g., pancreatic cell, is directly exposed to the nucleic acid or nucleic acid-carrying vectors, or indirect, in which case, cells, e.g., pancreatic cells, are first transformed with the nucleic acids in vitro, then transplanted into the patient. These two approaches are known, respectively, as in vivo or ex vivo gene therapy.

In certain embodiments, the nucleic acid may be directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. (1987); 262:4429-4432). The nucleic acid-ligand complexes can also be formed in which the ligand includes a fusogenic viral peptide to disrupt endosomes, allowing the nucleic acid to avoid lysosomal degradation. In addition, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA (1989); 86:8932-8935; Zijlstra et al., Nature (1989); 342:435-438).

In certain embodiments, a viral vector that contains a nucleic acid TALE fusion protein epigenetic modifier can be used. For example, a retroviral vector can be used (see Miller et al., Meth. Enzymol. (1993); 217:581-599). These retroviral vectors contain the components necessary for the correct packaging of the viral genome and integration into the host cell DNA. More detail about retroviral vectors can be found in Boesen et al., Biotherapy (1994); 6:291-302. Other references illustrating the use of retroviral vectors in gene therapy are: Anson, Genet Vaccines Ther 13; 2(1):9 (2004); Clowes et al., J. Clin. Invest. (1994); 93:644-651; Kiem et al., Blood (1994); 83:1467-1473; Salmons and Gunzberg, Human Gene Therapy (1993); 4:129-141; and Grossman and Wilson, Curr. Opin. in Genetics and Devel. (1993); 3:110-114.

In certain embodiments, adenoviruses are especially attractive vehicles for delivering genes. Adenoviruses have the advantage of being capable of infecting non-dividing cells. Kron and Kreppel, Curr Gene Ther 12(5):362-73 (2012) and Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995).

In certain embodiments, adeno-associated virus (AAV) can be used (Zhong et al. J Genet Syndr Gene Ther January 10; S1. pii:008; High, K A, Blood, 120(23):4482-7 (2012); Walsh et al., Proc. Soc. Exp. Biol. Med. 204:289-300 (1993); U.S. Pat. No. 5,436,146). In certain embodiments, AAV vectors are used. Vectors that can be used in gene therapy are discussed below in detail below.

Another approach to gene therapy involves transferring a gene to pancreatic cell in tissue culture by such methods as electroporation, lipofection, calcium phosphate mediated transfection, or viral infection. Usually, the method of transfer includes the transfer of a selectable marker to the pancreatic cells. The cells are then placed under selection to isolate those pancreatic cells that have taken up and are expressing the transferred gene. Those pancreatic cells are then delivered to a patient.

In certain embodiments, the nucleic acid can be introduced into cells, e.g., pancreatic cells, prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and can be used in accordance with the present disclosure, provided that the necessary developmental and physiological functions of the recipient cells are not disrupted.

The resulting recombinant cells can be delivered to a patient by various methods known in the art. The amount of cells envisioned for use depends on the desired effect, patient state, etc., and can be determined by one skilled in the art.

Recombinant cells can also be used in gene therapy, where nucleic acid sequences encoding a TALE fusion protein epigenetic modifier, are introduced into the cells such that they are expressible by the cells or their progeny, and the recombinant cells are then administered in vivo for therapeutic effect. For example, stem or progenitor cells can be used. Any stem and/or progenitor cells which can be isolated and maintained in vitro can potentially be used (see e.g. PCT Publication WO 94/08598; Porada and Porada, J. Genet Syndr Gene Ther., May 25; S1. p 11:011 (2012); Stemple and Anderson, Cell 71:973-985 (1992); Rheinwald, Meth. Cell Bio. 21A:229 (1980); and Pittelkow and Scott, Mayo Clinic Proc. 61:771 (1986)). Specific promoters can be used for targeting of pancreatic α-cells. For example, the promoter of the a-cell specific pre-proglucagon gene can be used (Nian M, Gu J, Irwin D M, Drucker D J. Am. J. Physiol. Regul. Integr. Comp. Physiol. 2002 January; 282(1): R173-83).

In certain embodiments, the TALE fusion protein epigenetic modifier can be delivered to cells using aptamers. Examples of drug delivery using cell-specific aptamers have been previously described (see Subramanian N et al., Mol Vis. 2012, 18: 2783-95; Li L L, Yin Q, Cheng J, Lu Y. Adv. Healthc. Matter. 2012 Sep. 1(5): 567-72; Zhou J, Bobbin M L, Burnett J C, Rossi J J, Front. Genet. 2012: 3:234, the contents of which are incorporated herein by reference)

### 4. Vectors

The terms “vector” and “expression vector” mean the vehicle by which a DNA or RNA sequence (e.g., a foreign gene) can be introduced into a host cell, so as to transform the host and promote expression (e.g., transcription and translation) of the introduced sequence. Vectors include plasmids, phages, viruses, etc.; they are discussed in greater detail below. A “therapeutic vector” as used herein refers to a vector which is acceptable for administration to an animal, and particularly to a human.

Vectors typically include the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA can be from the same gene or from different genes, and can be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET plasmids (Invitrogen, San Diego, Calif.), pCDNA3 plasmids (Invitrogen), pREP plasmids (Invitrogen), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g., antibiotic resistance, and one or more expression cassettes.

Suitable vectors include viruses, such as adenoviruses, adeno-associated virus (AAV), vaccinia, herpesviruses, baculoviruses and retroviruses, parvovirus, lentivirus, bacteriophages, cosmids, plasmids, fungal vectors, naked DNA, DNA lipid complexes, and other recombination vehicles typically used in the art which have been described for expression in a variety of eukaryotic and prokaryotic hosts, and can be used for gene therapy as well as for simple protein expression.

Lentiviral vectors have been reported to deliver genes to cells, e.g., pancreatic cells, efficiently and permanently, (Ravet et al. Cancer Gene Therapy 2010 May; 17(5):315-24). Lentiviral vectors are described in, for example, Choi et al (2001, Stem Cells 2001; 19(3):236-46) or in U.S. Pat. No. 6,218,186.

Viral vectors, especially adenoviral vectors can be complexed with a cationic amphiphile, such as a cationic lipid, polyL-lysine (PLL), and diethylaminoethyldextran (DELAE-dextran), which provide increased efficiency of viral infection of target cells (See, e.g., PCT/US97/21496 filed Nov. 20, 1997, incorporated herein by reference). AAV vectors, such as those disclosed in Zhong et al., J. Genet Syndr Gene Therapy 2012 Jan. 10; S1. pii: 008, U.S. Pat. Nos. 5,139,941, 5,252,479 and 5,753,500 and PCT publication WO 97/09441, the disclosures of which are incorporated herein, are also useful since these vectors integrate into host chromosomes, with a minimal need for repeat administration of vector. For a review of viral vectors in gene therapy, see McConnell et al., 2004, Hum Gene Ther. 15(11):1022-33; Mccarty et al., 2004, Annu Rev Genet. 38:819-45; Mah et al., 2002, Clin. Pharmacokinet. 41(12):901-11; Scott et al., 2002, Neuromuscul. Disord. 12(Suppl 1):523-9.

### 5. Pharmaceutical Compositions

In certain embodiments, the present disclosure provides pharmaceutical compositions which include an epigenetic modifier, alone or in combination with at least one other agent, such as a stabilizing compound or additional therapeutic agent, and can be administered in any sterile, biocompatible pharmaceutical carrier, including, but not limited to, saline, buffered saline, dextrose, and water. The composition can be in a liquid or lyophilized form and includes a diluent (Tris, citrate, acetate or phosphate buffers) having various pH values and ionic strengths, solubilizer such as Tween or Polysorbate, carriers such as human serum albumin or gelatin, preservatives such as thimerosal, parabens, benzylalconium chloride or benzyl alcohol, antioxidants such as ascrobic acid or sodium metabisulfite, and other components such as lysine or glycine. Selection of a particular composition will depend upon a number of factors, including the condition being treated, the route of administration and the pharmacokinetic parameters desired. A more extensive survey of components suitable for pharmaceutical compositions is found in Remington's Pharmaceutical Sciences, 18th ed. A. R. Gennaro, ed. Mack, Easton, Pa. (1980).

In certain embodiments, the methods and compositions of the present disclosure find use in treating diabetes. Peptides can be administered to the patient intravenously in a pharmaceutically acceptable carrier such as physiological saline. Standard methods for intracellular delivery of peptides can be used (e.g., delivery via liposome). Such methods are well known to those of ordinary skill in the art. The formulations of the present disclosure, are useful for parenteral administration, such as intravenous, subcutaneous, intramuscular, and intraperitoneal. Therapeutic administration of a polypeptide intracellularly can also be accomplished using gene therapy. The route of administration eventually chosen will depend upon a number of factors and can be ascertained by one skilled in the art.

In certain embodiments, the pharmaceutical compositions of the present disclosure can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in the present disclosure include, in certain embodiments, compositions where the active ingredients are contained in an effective amount to achieve the intended purpose. The amount will vary from one individual to another and will depend upon a number of factors, including the overall physical condition of the patient, e.g., severity and the underlying cause of the diabetes.

In certain embodiments, the formulations of the present disclosure can be administered for prophylactic and/or therapeutic treatments. For example, in alternative embodiments, pharmaceutical compositions of the present disclosure are administered in an amount sufficient to treat, prevent and/or ameliorate a disease, e.g., diabetes. As is well known in the medical arts, dosages for any one patient depends upon many factors, including stage of the disease or condition, the severity of the disease or condition, the patient's size, body surface area, age, the particular compound to be administered, sex, time and route of administration, general health, and interaction with other drugs being concurrently administered.

Accordingly, in certain embodiments, epigenetic modifiers can be administered to a patient alone, or in combination with one or more other drugs, nucleotide sequences, lifestyle changes, etc. used in the treatment or prevention of disease, e.g., diabetes, or symptoms thereof (for example, in the case of diabetes, insulin pancreatic stimulators, insulin sensitizers and exogenously supplied insulin or insulin analogs) or in pharmaceutical compositions where it is mixed with excipient(s) or other pharmaceutically acceptable carriers.

In certain embodiments, the pharmaceutically acceptable carrier is pharmaceutically inert. In certain embodiments of the present disclosure, epigenetic modifiers can be administered alone to individuals subject to or suffering from a disease, e.g., diabetes. The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the present disclosure are correct and appropriate.

Single or multiple administrations of formulations can be given depending on the dosage and frequency as required and tolerated by the patient. In certain embodiments, the formulations should provide a sufficient quantity of active agent to effectively treat, prevent or ameliorate the disease to be treated, e.g., diabetes, or symptoms or complications thereof as described herein. For example, in certain embodiments, an exemplary pharmaceutical formulation for oral administration can be in a daily amount of between about 0.1 to 0.5 to about 20, 50, 100 or 1000 or more μg per kilogram of body weight per day of protein. In certain embodiments, dosages are from about 1 mg to about 4 mg per kg of body weight per patient per day of protein are used. For example, in certain embodiments, a therapeutically effective amount of a polypeptide of this disclosure is a dosage of between about 0.025 to 0.5 milligram per 1 kilogram of body weight of the patient; or, a therapeutically effective amount is a dosage of between about 0.025 to 0.2 milligram, or 0.05 to 0.1 milligram, or 0.075 to 0.5 milligram, or 0.2 to 0.4 milligram, of the compound per 1 kilogram of body weight of the patient. In certain embodiments, a single dose is sufficient to achieve the desired results.

In certain embodiments, the epigenetic modifiers of the present disclosure are administered once, twice, or three times per week, by intravenous (IV) or subcutaneous (SC) injection to reach a suggested target therapeutic endpoint. Once the target has been achieved, a maintenance dosing schedule is established which will vary depending upon the patient.

Toxicity and therapeutic efficacy of such compounds can be determined by standard pharmaceutical procedures in cell cultures or experimental animals (LD50, the dose lethal to 50% of the population; and ED50, the dose therapeutically effective in 50% of the population). The dose ratio between toxic and therapeutic effects is the therapeutic index, and it can be expressed as the ratio LD50/ED50. Compounds that exhibit large therapeutic indices are advanageous in certain embodiments. The data obtained from these cell culture assays and additional animal studies can be used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The following examples are offered to more fully illustrate the present disclosure, but are not to be construed as limiting the scope thereof.

### EXAMPLES

### Example 1

### Epigenomic Plasticity Enables Human Pancreatic α- to β-Cell Reprogramming

Increasing the number of insulin-producing β-cells while decreasing the number of glucagon-producing α-cells, either in vitro in donor pancreatic islets before transplantation into type 1 diabetics, or in vivo in type 2 diabetics, is a promising therapeutic avenue.

Epigenetic studies have shown that manipulation of rodent histone acetylation signatures can alter embryonic pancreatic differentiation and composition (3, 4). Recent studies in rodent models have indicated that under extreme conditions, such as enforced ‘paired box gene 4’ (Pax4) or ‘pancreatic and duodenal homeobox gene 1’ (Pdx1) over-expression, or near complete β-cell ablation, α-cells can be reprogrammed towards the β-cell fate (5-7). However, the molecular basis of this reprogramming potential is unknown. The results contained herein address whether epigenetic mechanisms play a role in this process, and if α-cells exist in a metastable epigenetic state that facilitates their reprogramming.

While previous studies have characterized histone methylation signatures, open chromatin sites, and CTCF (CCCTC-binding factor)-binding sites of whole human islets (8-10), cell-type-specific epigenetic signatures and RNA-Seq analysis of human pancreatic cells have not been determined.

A previously published sorting strategy of human islets to obtain cell populations highly enriched for α-, β-, and exocrine (duct and acinar) cells (11) was employed to assess their cell-type-specific histone methylation profiles. Investigation of the trimethylation of the fourth amino acid (lysine) on histone H3 (H3K4me3) and the H3K27me3 mark was chosen because of their known association with cell fate determination and important roles in the regulation of transcription (12-14). While the H3K4me3 modification is associated with gene activation, the H3K27me3 mark is linked to repression of transcription (14). The presence of both marks at the same gene is referred to as a ‘bivalent mark’, and is more common in pluripotent, undifferentiated cells than in terminally differentiated cells (12). The bivalent mark keeps genes poised in an activatable state and is usually resolved during differentiation.

The human pancreatic cell-type-specific analysis of the activating and repressive histone methylation landscape, and the parallel determination of the complete transcriptome using RNA-Seq, is important in understanding the diabetes to facilitate treatment. Detailed analysis of these histone marks, their integration with gene expression data, and in vitro manipulation of their epigenomic signature provides a new pathway to reprogram α- to β-cells, as described herein.

Methods

FACS Sorting of Human Islet Cells and Experimental Setup

Fluorescent activated cell sorting (FACS) was performed on dispersed human islets using cell surface antibodies (1:20) and secondary antibodies (1:200, 115-116-075, 115-135-164, Jackson-ImmunoResearch) as described (11) to obtain cell populations highly enriched for α-, β- and exocrine (duct and acinar) cells. Total RNA was isolated from whole islets, and sorted α-, β-, and exocrine cells using the Ambion® mirVana™ miRNA Isolation Kit (AM1560), reverse transcribed to cDNA using SuperScript™ II reverse transcriptase (Invitrogen) and mRNA levels (normalized to β-Actin, GAPDH) were measured by qPCR analysis. Sample purity of the sorted α- and β-cell populations was calculated as percentage of contamination by the opposite cell type.

Assessment of Sample Purity

Sample purity of the sorted α- and β-cell populations was calculated as percentage of contamination by the opposite cell type using qPCR expression values for cell-specific markers as follows:

% α-cell contamination (in β-cell population)=purity(α)×[Gcg mRNA (β-cells)/Gcg mRNA (α-cells)]

% β-cell contamination (in α-cell population)=purity(β)×[lns mRNA (α-cells)/lns mRNA (β-cells)]

The maximum purity is 100%, and therefore an upper bound can be computed on the contamination without knowing the purity of the other cell population:

% α-cell contamination (in β-cell populations)≦[Gcg mRNA (β-cells)/Gcg mRNA (α-cells)]

% β-cell contamination (in α-cell population)≦[lns mRNA (α-cells)/lns mRNA β-cells)]

ChIP-Seq Analysis

Chromatin immunoprecipitation and preparation of ChIP-Seq libraries was performed on individual cell sorts for each cell type and donor (H3K4me3: Abcam 8580, H3K27me3: Upstate 07-449).

Libraries for H3K4me3, H3K27me3, and input were sequenced on an Illumina GA-IIx to 36 bp. Reads were aligned to hg18 using ELAND. Up to two mismatches were allowed and only reads with a best alignment to a single location were used for further analysis. Enrichment of H3K4me3 was performed on individual donors and cell types using GLITR (FDR=5%) (32) and a pool of human input from various tissues. GLITR was also run on islet cell input samples and any peaks identified in the inputs were filtered from the H3K4me3 enriched regions. Enriched regions in the H3K27me3 data were identified using the program STAR (33) with a sliding window of 5,000 bp, step size of 1,000 bp, and a FDR=0.5%. To ensure fair comparison, H3K4me3 and H3K27me3 profiles from CD4-positive T-cells (23) were determined using the same algorithms and settings.

Histone Modification Classification

To classify genes as H3K4me3-only, H3K27me3-only, bivalent, or unmarked pools of all regions identified as enriched for either H3K4me3 or H3K27me3 for the individual cell types were considered. Overlapping regions for the same mark were merged to produce a set of regions enriched for that mark in at least one donor. A gene was considered to have the H3K4me3 mark when a merged enriched region overlapped the region 2 kb downstream of the transcription start site, by at least 500 bp. A gene was considered to have the H3K27me3 mark when a merged enriched region overlapped the whole gene by at least 500 bp. A gene was considered bivalently marked if it was marked by both H3K4me3 and H3K27me3 using these definitions.

Summarizing multiple transcript annotations for each gene was done using these rules: 1. Genes with any transcript marked bivalent are considered bivalent; 2. Genes with a transcript marked H3K4me3, and another transcript marked H3K27me3 are considered “Ambiguous”; 3. Genes with at least one transcript marked H3K4me3, and no transcripts marked H3K27me3, are considered “H3K4me3-only”; 4. Genes with at least one transcript marked H3K27me3, and no transcripts marked H3K4me3, are considered “H3K27me3-only”; 5. All remaining genes are considered “Unmarked”.

A very small number (α-cells: 25 genes, β-cells: 30 genes, exocrine cells: 59 genes out of 21,457 genes) of genes were considered “Ambiguous” by these criteria, and were excluded from subsequent analysis.

Quantitative ChIP-Seq Analysis

Heatmap analysis was performed to assess whether the individual samples were correlated and equally contributing to the calls. H3K4me3 heatmaps were generated as follows. For each TSS in RefGene, the number of reads were counted from each sample in the region 2 kb downstream of the TSS (same region used for assigning H3K4me3 GLITR peaks to genes), and normalized to reads per million per kb (RPKM). Alternate TSSs for the same gene were combined by taking the maximum normalized signal within each sample. H3K4me3 RPKM signals for each gene were then transformed to the log 2 scale, and median normalized across samples. For each sorted cell population, normalized signals were plotted in a heatmap, with columns corresponding to individual samples, and rows corresponding to genes. The row order was determined by first grouping the genes called as H3K4me3 for the corresponding cell population (GLITR analysis, indicated by the solid blue bar), and then ordering genes by the average signal across all individual samples. H3K27me3 heatmaps were generated by a similar method, except that reads were counted and normalized across the entire gene body, and the H3K27me3 gene calls used for row grouping were based on STAR calls (indicated by the solid red bar). P-values were determined for the correlation between each pair of samples using the cor.test function in R. Normalized RPKM values were then averaged together for samples of the same cell type and used for comparisons of histone mark enrichments between cell-specific expression groups.

RNA-Sequencing

After total RNA extraction (see above), RNA-Seq libraries were prepared from sorted alpha, beta, and exocrine cells. The detailed protocol can be found on http://ngsc.med.upenn.edu (Lab protocol, Library preparation for RNA-Seq from total RNA). Libraries were single-end sequenced to 100 bp on an Illumina hiSeq2000. Reads from ribosomal RNA and genomic repeats were identified by aligning the 5′ 50 bp of each read to ribosomal sequences and the human repeats in RepBase (version 14.10) using Bowtie (Langmead B., Curr. Protoc. Bioinformatics. 201, December Chapter 11: Unit 117) and allowing for up to three mismatches. The remaining reads were processed with RUM (Grant et al., Bioinformatics 2011. Sep. 15, 27(18): 2518-28) and aligned to the set of known transcripts included in RefSeq, UCSC known genes, and ENSEMBL transcripts, and the human genome (hg18, NCBI build 36.1). Transcript-, exon-, and intron-level quantification was done using only the uniquely aligning reads. This process was repeated separately for each cell type and donor.

Gene Expression Analysis

To analyze global gene expression profiles from each sample, RNA-seq read counts aligning to exons of mRNA transcripts in RefSeq were extracted, and these values were normalized to total uniquely aligning reads and transcript length (RPKM). Data for individual genes was summarized by selecting a “representative transcript” with the highest RPKM value for each gene symbol (total=18,822 genes). Gene-level data was then quantile-normalized across all samples to remove bias from the variable expression levels of highly expressed genes, e.g., insulin, glucagon, and highly expressed ncRNAs, which dampen the relative signal of all other genes. Finally, quantile-normalized gene expression values were averaged together for samples of the same cell type to produce a single average gene profile for each cell type. These values were used for all subsequent analyses, including expression comparisons for individual genes of interest.

For the heatmap of cell-type specific expression, all genes below 0.5 RPKM normalized average expression were removed in all three cell types. The remaining 14,003 genes were each normalized to percentage of their maximum expression across the three cell type to focus the analysis on the cell-type specificity of each gene, rather than the absolute expression level. These values were clustered using the R package ‘mclust’ v4.0 (Yeung, et al. 2001). K=20 was chosen as the number of clusters, as Bayesian Information Content did not improve substantially with additional clusters. Clusters were grouped together based on their overall cell-type specificity.

For Principal Component Analysis (PCA) of the cell types, the quantile normalized average values (including those below 0.5 RPKM) were used to build three principle components (linear transformations of the gene expression profile in each sample), using the ‘prcomp’ function in R. The first two principle components captured 99% of the variance across the cell types, and were used to map the average and individual replicate profiles onto a 2D plot.

For the comparison between marked and unmarked genes in the same cell type, the average quantile-normalized values for the representative transcript of each gene was used, and further transformed these values to log 2 scale to visualize expression for both low and high-expression genes. For each group of genes based on histone marks in a single cell type, a one-sample Wilcoxon test (R ‘wilcox.test’ function) was used to assess whether the distribution of expression values was significantly shifted above or below 0 on the log 2 scale (corresponding to RPKM=1 on the non-log scale). A shift above this value indicates a preference for highly expressed genes, while a shift below this value indicates a preference for weakly or non-expressed genes.

Identification of Cell Type-Specific Novel Transcripts

A candidate was selected for regions for novel transcription based on the genomic coverage data output from RUM (number of RNA-seq reads overlapping each base in the genome). Specifically, all regions in each alpha or β-cell RNA-seq sample where the read coverage was >1 RPM continuously for at least 200 bp was selected. These regions were then merged together into a master set of candidates regions, and filtered out all regions that overlapped any known exon in the feature quantification table used by RUM (contains RefSeq, UCSC Known Genes, and Ensembl transcripts) to rule out all known transcribed regions. This method still allows for any candidate region that falls entirely in a known intronic region, as some lncRNAs have been observed in these regions. Any candidate region overlapping a repeat region from the UCSC Repeat Masker track were removed as these regions are more likely to be mapping or amplification artifacts. The remaining candidate regions were compared to the H3K4me3 peak calls from both alpha and β-cells, and limited the subsequent analysis to only those regions within 5 kb of an H3K4me3 peak, as this histone mark provides additional evidence of active transcription. The candidate selection process above resulted in 317 candidate regions with evidence for active transcription, based on both the RNA-seq and H3K4me3 data, which do not overlap known transcripts or repeat regions. Expression levels of each of these candidate regions were then quantified the by counting the number of uniquely aligning reads overlapping these regions, normalized to transcript length and total number of reads in each samples (RPKM). The mean normalized expression value was computed for each novel transcript in the three α-cell RNA-seq samples and the three β-cell RNA-seq samples, and computed a fold-change between these two cell-types. Alpha-specific transcripts were defined to be those with >2× higher mean expression in α-cells, and beta-specific transcripts were defined to be those with >2× higher mean expression in β-cells.

Histone Modification Analysis of Functional Gene Categories within Alpha- and Beta-Cell Signature Genes

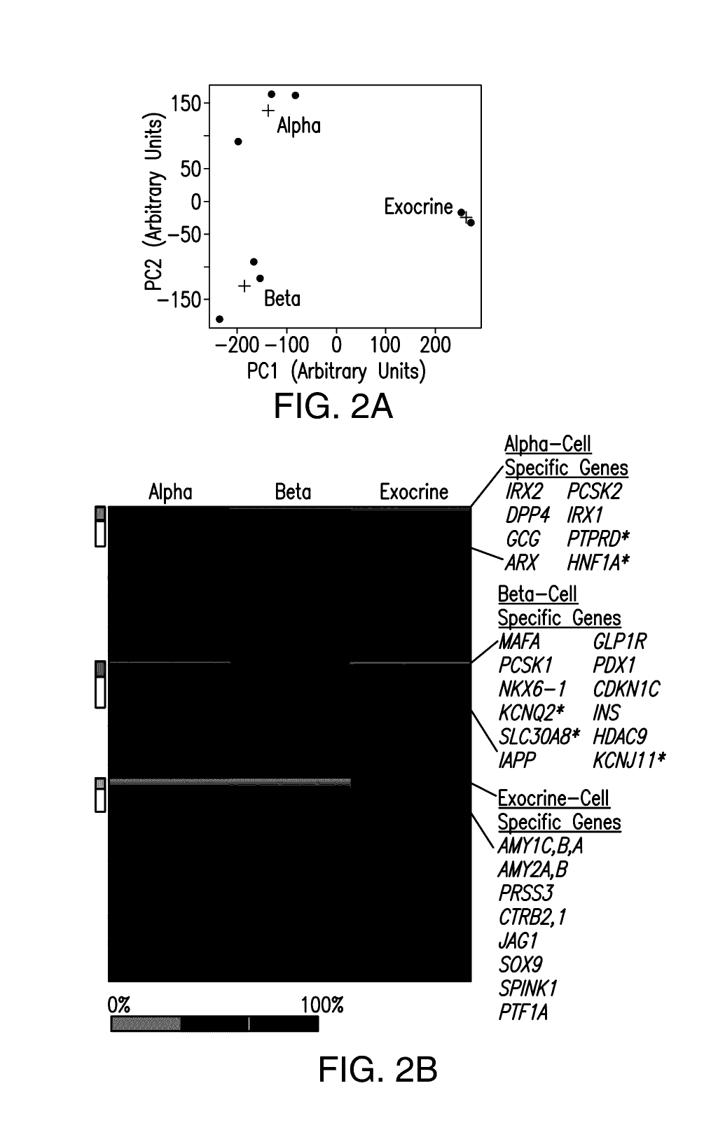

Lists of strongly cell-type specific genes (FIG. 2B, indicated by darker portion of bars) were analyzed for their likely gene function using DAVID. The two functional gene categories ‘ion transport’ and ‘regulation of transcription, DNA dependent’ comprised similar numbers of genes (ion transport: 29 alpha-specific and 34 beta-specific genes; regulation of transcription, DNA-dependent: 29 alpha-specific and 31 beta-specific genes). These genes were further analyzed for their histone modification status in alpha-, beta-, and exocrine cells and the percentage of histone modification (bivalent, monovalent H3K4me3, monovalent H3K27me3, none of the above) within each gene group was calculated.

Computational Analysis

The GLITR algorithm (32) was utilized for detection of H3K4me3 enrichment and the STAR algorithm (33) for detection of H3K27me3 enrichment, as the broad architecture of the repressive H3K27me3 mark requires more sensitive peak-calling algorithms for precise analysis (34).

Adox Treatment and Immunostaining of Human and Mouse Islets

Human islets and islets from GlucagonCre; Rosa26EYFP bigenic mice (Herrera et al., 2000; Srinivas et al., 2001) were cultured, treated with 50μM adenosine dialdehyde (Adox, Sigma A7154) for 72 h, FACS sorted as described previously (11), or prepared for immunostaining.

Immunofluorescent confocal analysis was performed as described previously (Gao et al., Genes Dev. 2010, Jun. 15, 24(12): 1295-305). Sections were blocked with CAS-Block (Invitrogen, 00-8120) and stained using guinea pig anti-insulin 1:500 (Abcam, ab7842), rabbit anti-glucagon 1:250 (Santa Cruz, sc13091), guinea-pig anti-Pdx1 1:1000 (gift from Dr. C. Wright), mouse anti-E-Cadherin 1:500 (BD Transduction Laboratories, 610181), goat anti-GFP (Abcam, ab6673), guinea pig Cy2, guinea pig Cy5, rabbit Cy3, mouse Cy2, mouse Cy5, goat Cy3 (1:500, Jackson-Immunoresearch) antibodies. For quantification of double PDX1+/GCG+ cells, Pdx1-, Glucagon-, E-Cadherin and DAPI staining were performed on untreated (control) and Adox-treated islets. All glucagon-positive cells and all double PDX1+/GCG+ cells were counted manually (8 slides) in each condition and the percentage of PDX1+/GCG+ double-positive cells of all glucagon-positive cells was calculated.

### Results

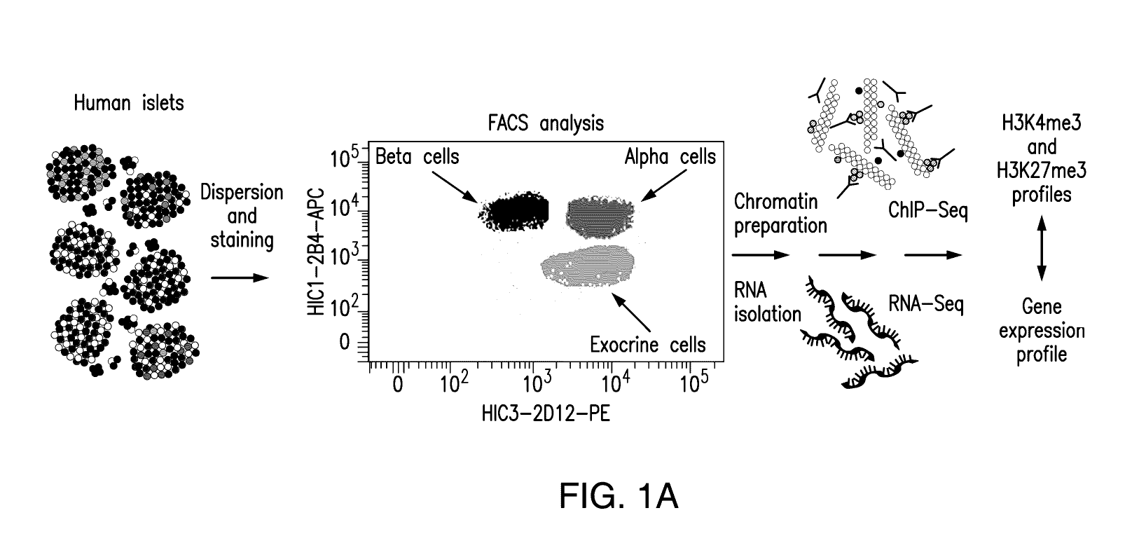

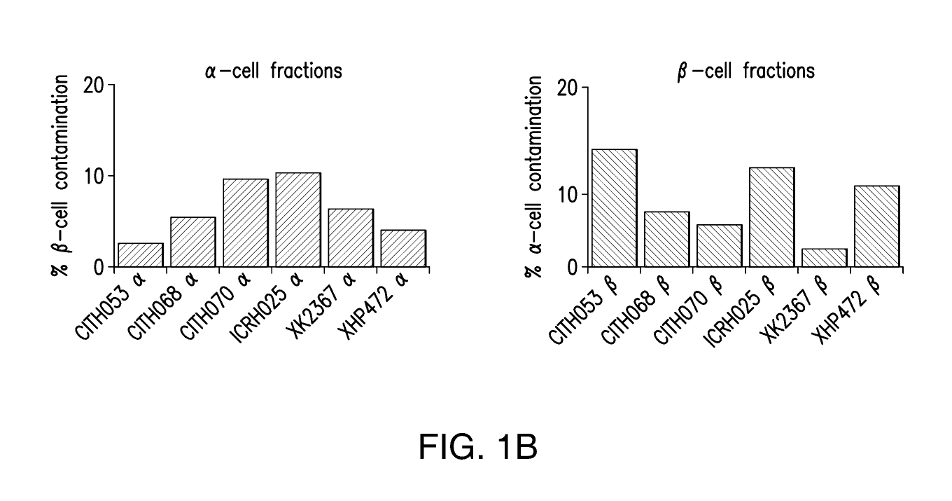

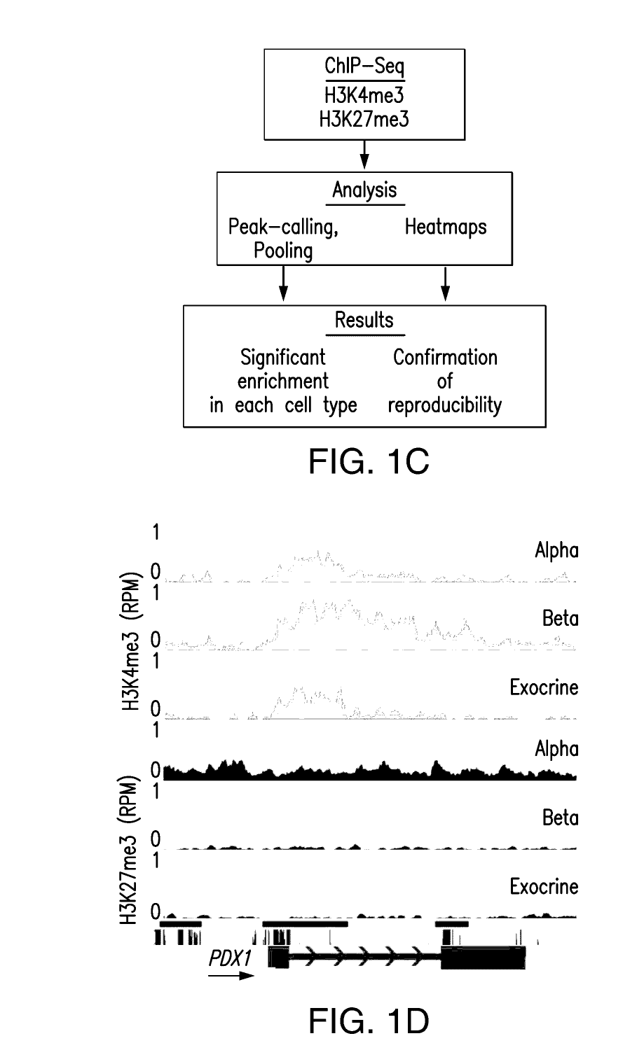

Human pancreatic islets from deceased organ donors (n=6, Table 1) were sorted into highly enriched α-, β-, and exocrine (duct and acinar) cell fractions using a cell surface antibody panel (11) and the additional antibody 2D12 (FIG. 1A). Sample purity of the sorted α- and β-cell populations was validated by qRT-PCR for relevant marker genes. The sample purity was calculated as percentage of contamination by the opposite cell type and found that the α- and β-cell fractions were on average 94% and 92% pure (FIG. 1B). Next, the transcriptomes and histone methylation profiles of the sorted cell fractions were determined by RNA-Seq and chromatin immunoprecipitation/ultra high-throughput sequencing (ChIP-Seq) (FIG. 1A). The histone methylation profiles of each donor and cell type were analyzed individually, the H3K4me3 and H3K27me3 calls of each cell type to obtain cell-type-specific histone methylation profiles were pooled, and validated this approach by confirming the enrichment calls and their low inter-individual variability in a heatmap analysis (FIG. 1C). As an example, the enrichment profiles for H3K4me3 and H3K27me3 for the diabetes gene PDX1 in α-, β-, and exocrine cells are shown in FIG. 1D. PDX1 is expressed in mature β-cells and at lower levels in exocrine cells, but not in α-cells (15, 16), which is clearly reflected by the histone modifications, with H3K4me3 enrichment in all cell fractions, but an additional, repressive H3K27me3 mark present only in α-cells. Thus, the PDX1 locus is marked monovalently by H3K4me3 in β- and exocrine cells, but carries a bivalent mark (H3K4me3 and H3K27me3) in α-cells.

TABLE 1
Islet Donor Information.

| | | | | Cause of | | |
|---|---|---|---|---|---|---|
| ID | Sex | BMI | Age | death | Race | Experiments |
| CITH053 | F | 24.5 | 49 | CVA, ICH | AA | H3K4me3, |
| | | | | | | RNA-Seq |
| CITH068 | M | 26.8 | 52 | HT | C | H3K4me3, |
| | | | | | | H3K27me3, |
| | | | | | | RNA-Seq |
| XHP472 | M | 25.9 | 52 | CVA | H | H3K4me3 |
| CITH070 | M | 29.2 | 28 | Anoxia | C | H3K4me3, |
| | | | | | | RNA-Seq |
| ICRH025 | M | 28.3 | 47 | CVA, ICH | AA | H3K27me3 |
| XK2367 | F | 22 | 54 | GSWH | C | H3K27me3 |
| CITH090 | M | 33.8 | 22 | CVA | C | Adox treatment of |
| | | | | | | cultured islets, IF |
| ZAN423 | M | 33 | 27 | HT | C | Adox treatment of |
| | | | | | | cultured islets, sort |
| ZIQ120 | F | 25.4 | 57 | CVA | H | Adox treatment of |
| | | | | | | cultured islets, IF |

Abbreviations: AA, African American; C, Caucasian; H, Hispanic; CVA, Cerebrovascular accident; GSWH, Gunshot wound to the head; HT, Head trauma; and ICH, Intracerebral hemmorage.

RNA-Seq analysis was performed to assess the genome-wide transcriptional landscape in the sorted cell populations, and to analyze the purity of the cell populations on a genome-wide scale. Principal component analysis showed that the sorted cell populations are distinct and that the replicates (n=3α, n=3β, n=2exocrine) cluster together tightly (FIG. 2A). Next, cluster analysis was performed to identify groups of genes with distinct expression patterns across cell types, to focus on the cell-type-specific transcriptional differences, and to classify α-, β-, and exocrine-specific signature genes. The results are presented in a heat map, in which the three cell populations are displayed in their respective columns, and clusters of α-, β-, or exocrine-specific signature genes were identified and are marked as boxes next to the heatmap (FIG. 2B). Among the α-cell-specific genes are, as expected, the α-cell-specific transcription factor ‘aristaless related homeobox’ (ARX) and the α-cell hormone glucagon (GCG). In addition, the enzymes ‘prohormone convertase 2’ (PCSK2) and ‘dipeptidyl peptidase-4’ (DPP4) are expressed specifically in α-cells, the latter an important target of the commonly used group of oral drugs for type 2 diabetes, the DPP4-inhibitors or gliptins. The exocrine-cell-specific genes include many digestive enzymes, their inactive precursors and their inhibitors, such as various amylase isoforms (AMY1A, AMY1B, AMY1C, AMY2A, and AMY2B), ‘pancreatic trypsinogen III’ (PRSS3), ‘chymotrypsinogen I and II’ (CTRB1 and CTRB2), and the trypsin inhibitor ‘serine peptidase inhibitor, kazal type 1’ (SPINK). In addition, ‘jagged 1’ (JAG1), the transcription factor SOX9, and the ‘pancreas-specific transcription factor 1a’ (PTF1A) are also among the exocrine-specific genes. The RNA-Seq based list of β-cell-enriched genes includes the β-cell-specific transcription factors MAFA, NKX6-1, and PDX1, as well as the β-cell hormone insulin (INS) and one of the key enzymes for its synthesis, ‘prohormone convertase 1’ (PCSK1). The β-cell specific cluster also included ‘histone deacetylase 9’ (HDAC9), which has previously been shown to be enriched in murine β-cells (4). Many genes identified were found in genome-wide association studies for non-autoimmune forms of diabetes (17) among the α-cell and β-cell-specific genes, such as the ‘hepatocyte nuclear transcription factor 1-alpha’ (HNF1A) and the ‘protein-tyrosine phosphatase delta’ (PTPRD) in α-cells, and the potassium channels KCNQ2 and KCNJ11, and the zinc transporter SLC30A8 in β-cells. See Branswig et al. J. Clin. Invest. 2013 123(3):1275-84 (expressly incorporated herein by reference), for the complete gene lists of α-, β-, and exocrine-specific clusters. In addition, the transcriptomes of the α-, β-, and exocrine cell populations are presented in Branswig et al. J. Clin. Invest. 2013 123(3):1275-84, including normalized expression values of every gene.

To extend the analysis of the human α- and β-cell specific transcription atlas, searches were performed for novel, cell-type-specific long non-coding transcripts. Long non-coding RNA molecules (lncRNAs) have been implicated as important developmental regulators, cell lineage allocators, and contributors to disease development (18). Recently described human islet lncRNAs were regulated during development and dysregulated in type 2 diabetic islets (19). Therefore, discovery of novel lncRNAs and evaluation of their function can provide insight into diabetes pathogenesis. Twelve β-cell specific, and five α-cell-specific non-coding transcripts were found, indicative of the valuable research resource represented by the unique transcriptome data (Table 2).

TABLE 2
Identification of non-coding human endocrine
cell-type specific transcripts.

| Region | Beta | Alpha | Beta/Alpha |
|---|---|---|---|
| chr22: 45828637-45828942 | 14.54 | 2.46 | 5.91 |
| chr15: 32834228-32834476 | 11.05 | 3.13 | 3.53 |
| chr7: 98085143-98085366 | 43.10 | 12.86 | 3.35 |
| chr7: 98085583-98085801 | 47.10 | 16.87 | 2.79 |
| chr16: 546970-547176 | 7.28 | 2.78 | 2.62 |
| chr7: 97681668-97681869 | 14.41 | 5.77 | 2.50 |
| chr22: 17401525-17401730 | 12.20 | 4.99 | 2.45 |
| chr7: 97680610-97680816 | 11.32 | 4.72 | 2.40 |
| chr22: 28447815-28448044 | 11.87 | 5.41 | 2.20 |
| chr10: 71888615-71888815 | 14.43 | 7.05 | 2.05 |
| chr12: 107438050-107438287 | 19.60 | 9.63 | 2.04 |
| chr2: 91141148-91141355 | 17.53 | 8.65 | 2.03 |
| chr10: 100010310-100010623 | 4.48 | 39.59 | −8.83 |
| chrX: 129343875-129344132 | 2.63 | 13.44 | −5.11 |
| chr1: 146614868-146615074 | 8.57 | 18.98 | −2.22 |
| chr13: 57102889-57103187 | 5.25 | 11.11 | −2.12 |
| chrX: 110229332-110229577 | 15.61 | 31.49 | −2.02 |

Analysis of the RNA-seq data revealed twelve β-cell specific and five α-cell specific non-coding RNA transcripts not previously annotated in the genome (>2× higher mean expression than the opposite cell type). Stringent filtering criteria included a minimum length of 200 bp with at least 1 RPM (read per million) coverage and removal of all regions overlapping a repeat region from the UCSC Repeat Masker track, which includes other non-coding RNAs, such as ribosomal RNAs, and small nucleolar RNAs. In addition, regions were required to be within 5 kb of a H3K4me3 peak for additional evidence for transcription.

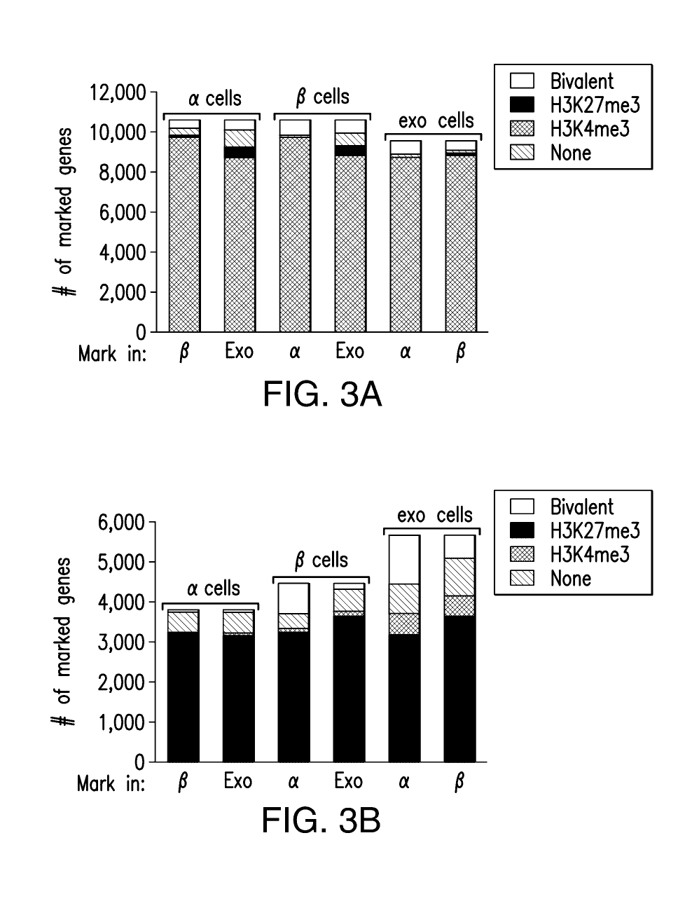

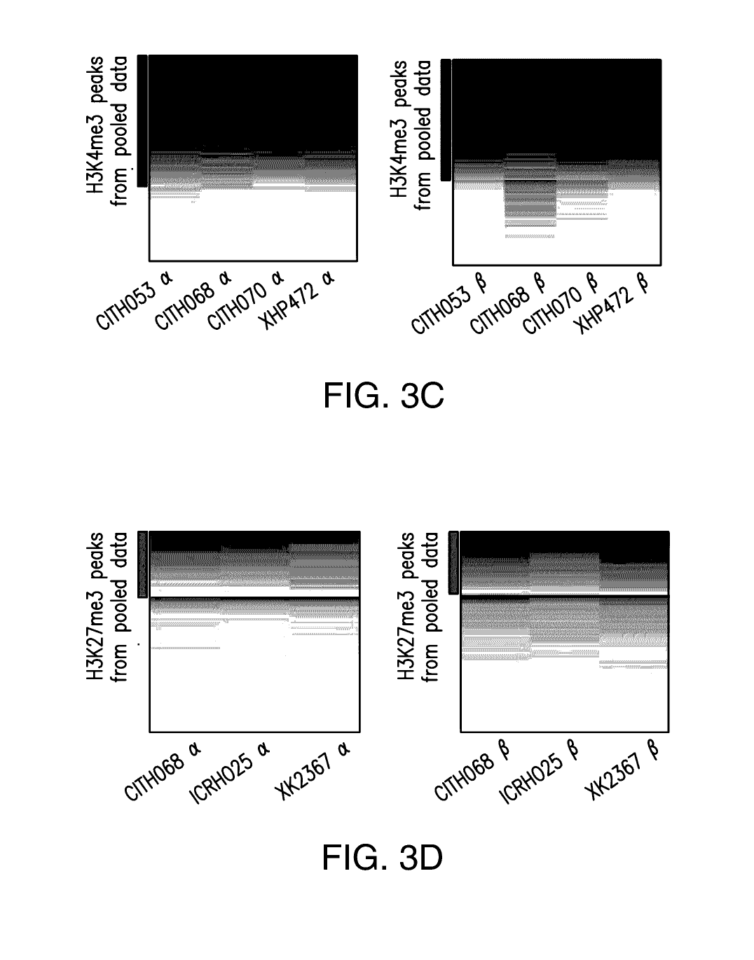

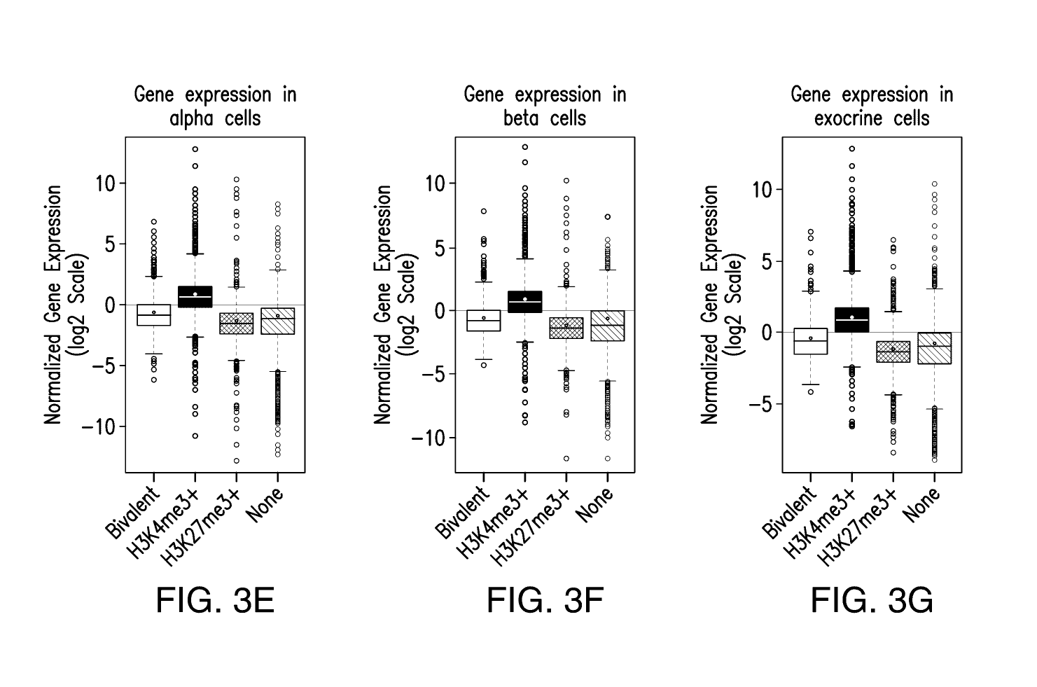

Next, genome-wide, monovalent histone modification landscapes of the sorted pancreatic cell populations was analyzed. Monovalent H3K4me3-enriched regions in α-, β-, and exocrine cells were identified and compared among the three cell types (FIG. 3A). The vast majority of monovalently H3K4me3-marked genes were shared among the three pancreatic cell lineages (83-95%), reflecting both their related function in protein secretion and common embryonic descent (FIG. 3A).

To investigate the landscape of repressive histone modifications, H3K27me3 ChIP-Seq analysis was performed, and monovalent H3K27me3 enrichment at 3,755 gene regions in α-, 4,420 gene regions in β-, and 5,628 gene regions in exocrine cells were detected (FIG. 3B). Similar to the H3K4me3 modification, a high degree of overlap of monovalently H3K27me3-marked genes between the three cell populations was found (73-83%, FIG. 3B). These H3K4me3- and H3K27me3-enrichment calls were validated by heat map analysis of the biological α- and β-cell replicates showing low inter-individual variability (FIGS. 3, C and D). The ‘box-and-whisker’ plots display the gene expression levels of bivalently marked, H3K4me3 marked, H3K27me3 marked and ‘unmarked’ genes in each cell population (FIGS. 3, E, F, and G). These data show that the bivalent, the monovalent H3K4me3, and the monovalent H3K27me3 enrichment calls were correlated genomewide with their respective mRNA levels at high statistical significance.

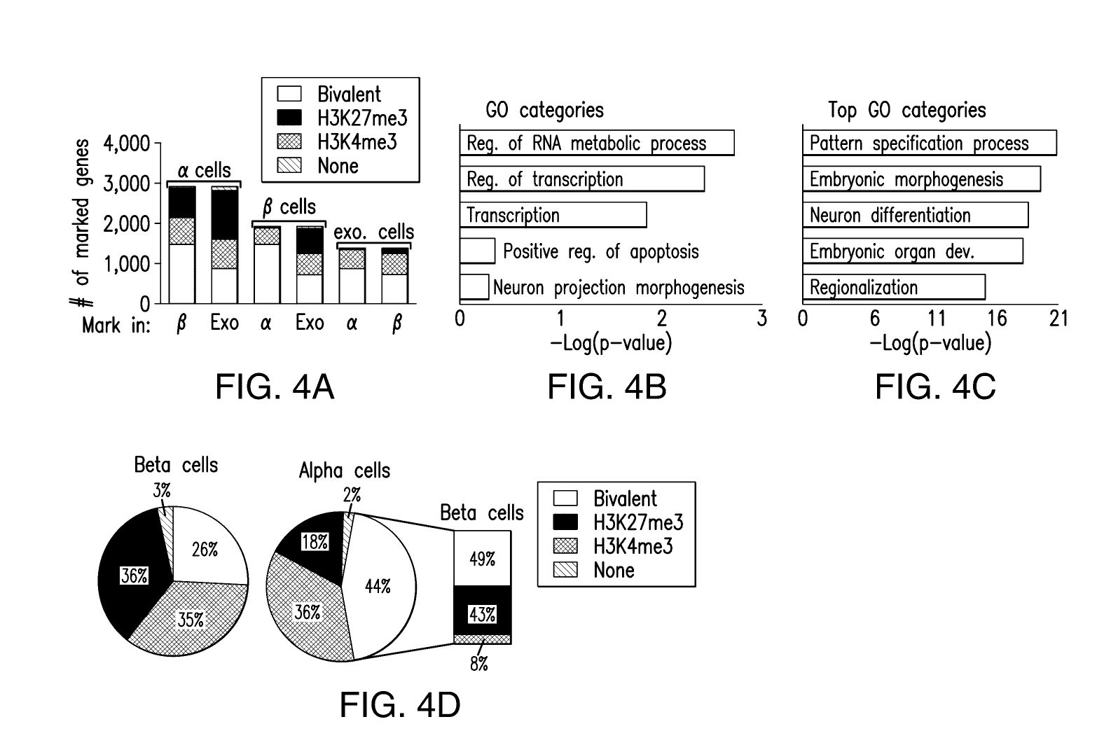

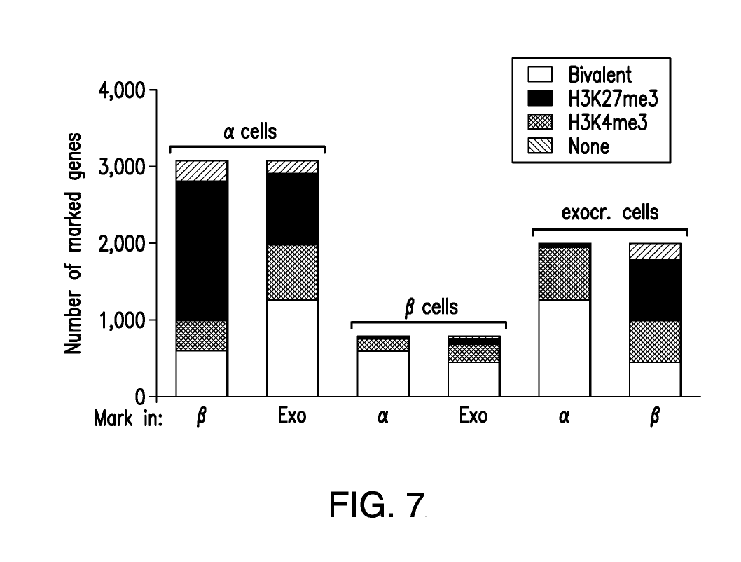

Bivalent marks have been observed to be common in undifferentiated cells, such as ES cells and pluripotent progenitor cells, and in most cases, one of the histone modification marks was lost during differentiation, accompanying lineage specification (12-14). Consequently, most genes in differentiated cells are marked by either H3K4me3 or H3K27me3, corresponding to an expressed or repressed state, respectively. Preserving the bivalent state in a subset of genes has been suggested to maintain higher plasticity (12-14). Interestingly, α-cells showed the highest incidence of bivalent marks (2,915 gene regions), followed by β-cells (1,914) and exocrine cells (1,368) (FIG. 4). As an internally controlled data set, the bivalent domains for all three cell types in one individual donor (CITH068) were detected and the higher number of bivalent marks in α-cells was confirmed (FIG. 7).

Analysis of genes carrying a bivalent mark in β-cells showed that the majority of these genes were also marked bivalently in α-cells (1474 genes, 77%), while 26 genes carried a monovalent H3K27me3 mark and 406 genes carried a monovalent H3K4me3 mark in α-cells. Gene ontology analysis (20, 21) of the 406 genes marked bivalently in 3-cells, but monovalently by H3K4me3 in α-cells, revealed three significantly enriched categories (FIG. 4B). However, no significantly enriched categories were identified in the 523 genes marked bivalently in exocrine, but monovalently by H3K4me3 in β-cells, or the 467 genes marked bivalently in exocrine, but monovalently by H3K4me3 in α-cells (data not shown). The histone methylation status (H3K4me3, H3K27me3, bivalent, none of the above) of every gene in α-, β-, and exocrine cells is provided in Branswig et al. J. Clin. Invest. 2013 123(3):1275-84.

Nearly half of the genes that displayed a bivalent mark in α-cells were marked only by H3K4me3 or H3K27me3 in β-cells (48%, 1,406 genes) (FIG. 4A, left bar) in contrast to 434 genes (22%) marked bivalently in β-cells, but monovalently in α-cells. Gene ontology analysis of genes that were marked bivalently in α-cells, but only by the repressive H3K27me3 mark in β-cells, displayed highly significant enrichment for genes involved in developmental processes (FIG. 4C and Table 3), indicating a more plastic epigenetic state of developmental genes in α-cells and a more fixed epigenetic condition in β-cells. To further confirm these observations, transcriptional regulators marked bivalently in human embryonic stem cells (hESC) (22) were compared to their histone profile in α- and β-cells. Only 26% of all transcriptional regulators marked bivalently in hESC were found to also show a bivalent mark in β-cells (FIG. 4D, left pie chart), while nearly half of them were marked bivalently in α-cells (44%) (FIG. 4D, right pie chart). Further analysis of genes marked bivalently in both hESC and α-cells showed that 43% of these genes were H3K27me3-modified in β-cells (FIG. 4D, inset). These findings support the enhanced epigenetic plasticity of human α-cells.

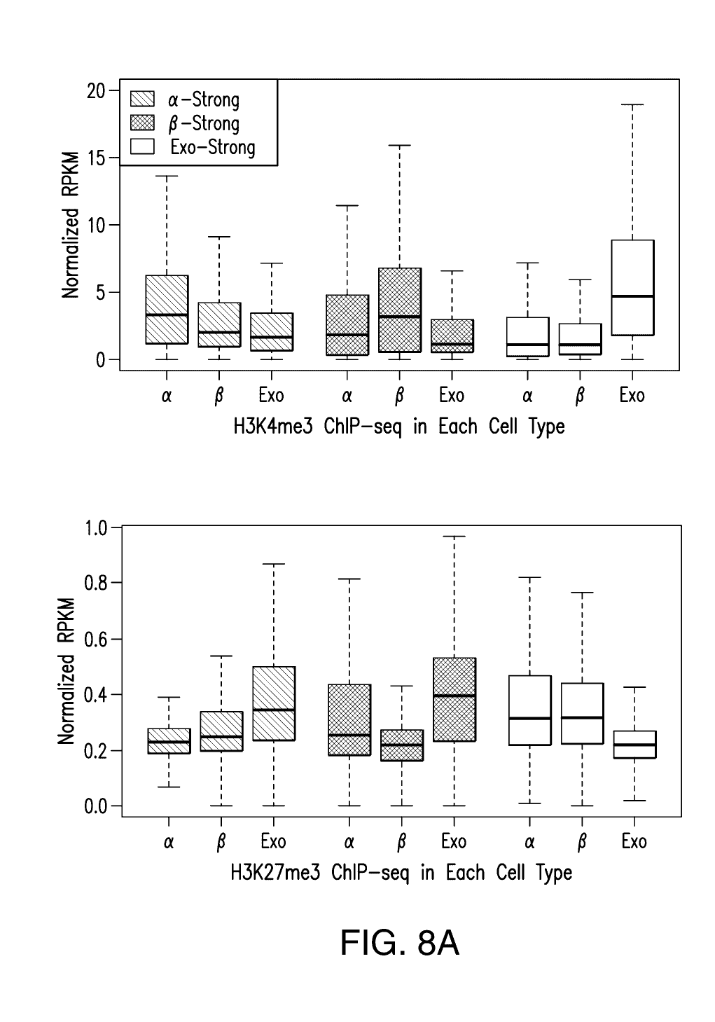

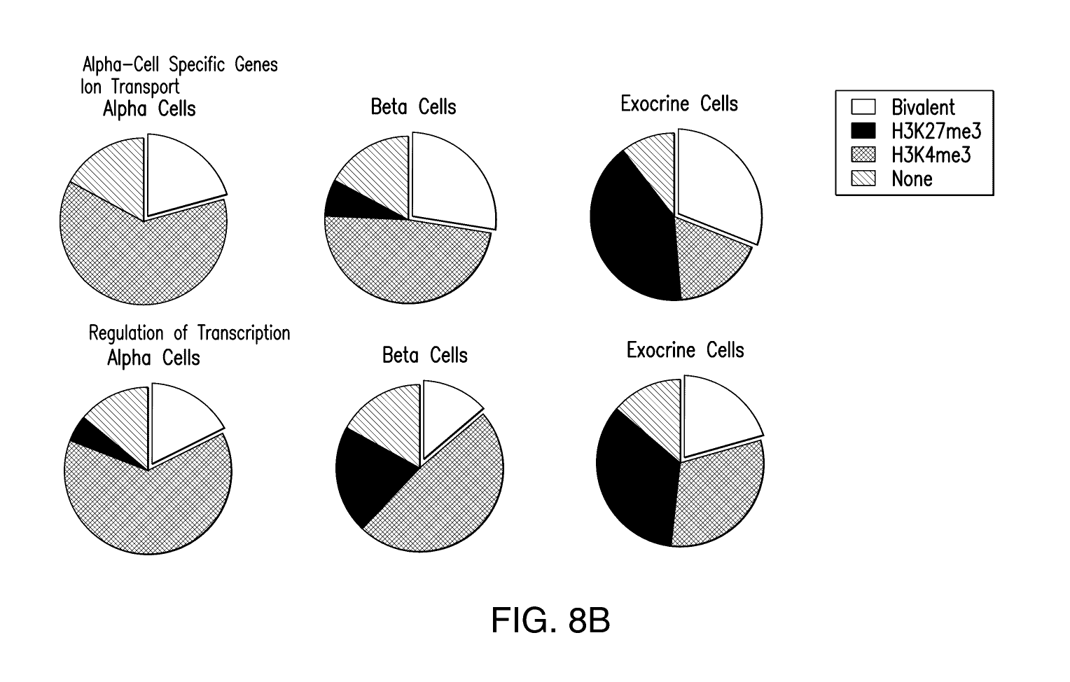

To extend these studies, the histone modification ChIP-seq data sets were integrated with the specific transcriptional signatures of α-, β-, and exocrine cells identified from the RNA-seq analysis (FIG. 2B). Quantitative analysis of H3K4me3 and H3K27me3 enrichment at α-, β-, and exocrine-specific signature genes in each of these cell types showed increased H3K4me3 levels in their respective signature gene group, as expected (FIG. 8). Interestingly, H3K27me3 levels of α-specific genes were comparable between α- and β-cells, whereas H3K27me3 levels of β-cell specific genes were increased in α-cells and decreased in β-cells, supporting higher prevalence of the H3K27me3 mark repressing β-signature genes in α-cells, rather than vice versa.

TABLE 3
Top GO categories from DAVID analysis on the genes marked
bivalently by H3K4me3 and H3K27me3 in α-cells and marked
monovalently by the repressive H3K27me3 mark in β-cells
show strong enrichment in developmentally relevant processes.

| | Gene | Benjamini p- | |
|---|---|---|---|
| GO category | count | value | FDR |
| Pattern specification process | 57 | 1.3E−21 | 9.0E−22 |
| Embryonic morphogenesis | 59 | 2.5E−20 | 3.3E−20 |
| Neuron differentiation | 69 | 2.4E−19 | 4.7E−19 |
| Embryonic organ development | 43 | 7.4E−19 | 2.0E−18 |
| Regionalization | 42 | 9.7E−16 | 3.2E−15 |
| Embryonic organ morphogenesis | 35 | 8.4E−16 | 3.4E−15 |
| Sensory organ development | 44 | 5.7E−15 | 2.7E−14 |
| Neuron development | 52 | 1.1E−13 | 5.9E−13 |
| Skeletal system development | 48 | 3.0E−12 | 1.8E−11 |
| Tube morphogenesis | 30 | 4.7E−12 | 3.2E−11 |
| Tube development | 39 | 5.8E−12 | 4.3E−11 |
| Cell morphogenesis involved in | 41 | 6.6E−12 | 5.3E−11 |
| differentiation | | | |
| Cell morphogenesis | 50 | 8.4E−12 | 7.3E−11 |
| Cell fate commitment | 30 | 4.0E−11 | 3.8E−10 |

Assessment of whether the increased H3K27me3 mark in α-cells was indicative of higher bivalency in functionally relevant β-cell specific genes, such as the transcriptional regulators that control cell-type specific gene expression, was performed. Functional gene categories within the strongly α- and β-specific genes were analyzed (dark colored boxes in FIG. 2B). Analysis of β-specific genes implicated in ‘ion transport’ (34 genes) showed that 29% of the genes were marked bivalently in α-, 15% in β-, and 6% in exocrine cells. Analysis of β-specific genes implicated in ‘regulation of transcription’ (31 genes) displayed a much higher percentage of bivalently marked genes in α-cells (42%) than in 13- or exocrine cells in reverse (16% and 13%), which was not observed in any of these functional categories in α-specific signature genes (FIG. 8B). Therefore, a large fraction of β-cell specific transcriptional regulatory genes are in a bivalent state in α-cells.

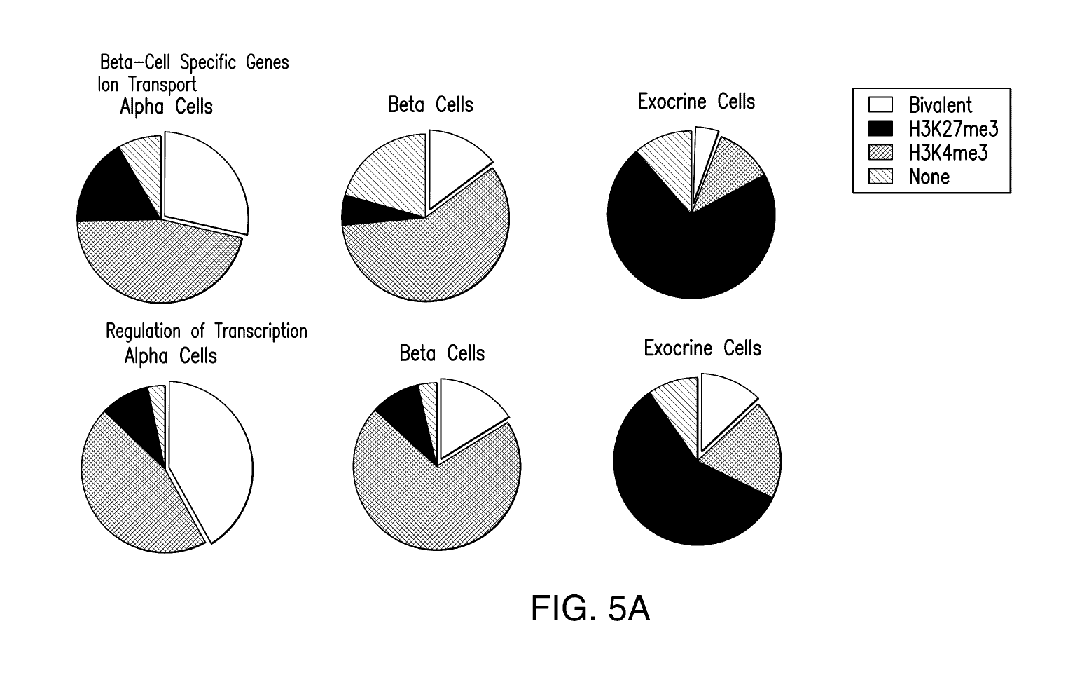

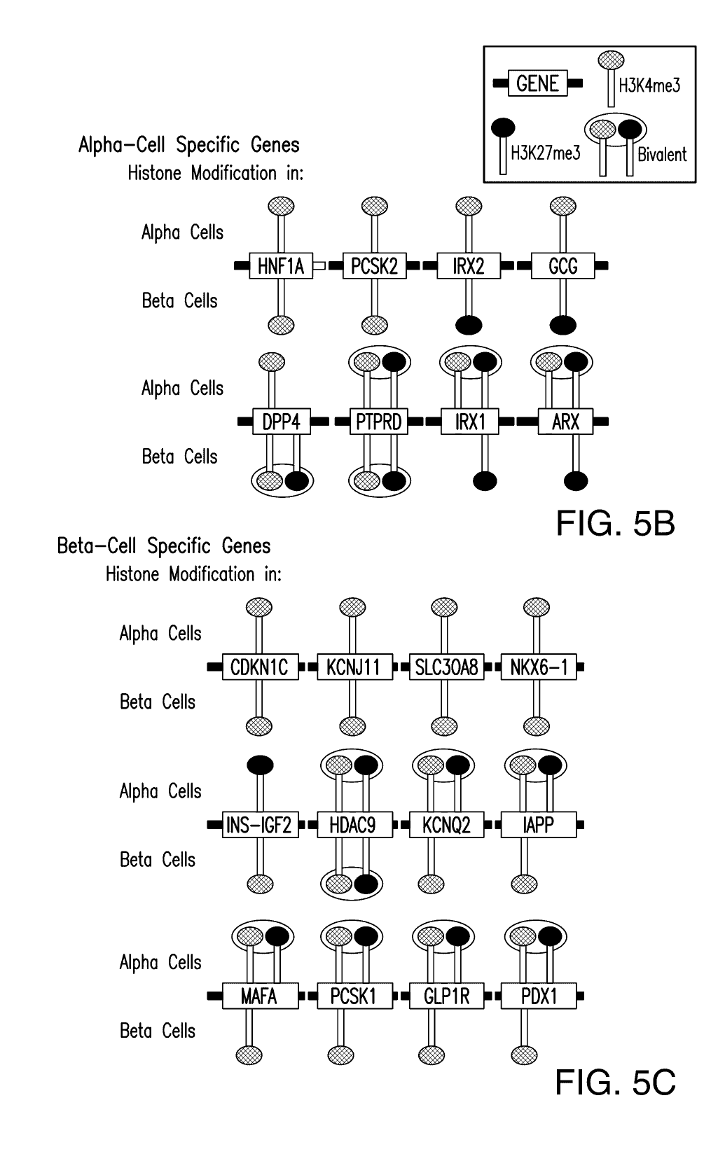

The cell-type specific analysis of the histone modification landscape was focused on α- and β-specific genes known to be important for pancreatic development and endocrine cell function. Analysis of the histone marks of α-specific genes in β-cells identified many as being marked monovalently by H3K4me3 (HNF1A, PCSK2) or by H3K27me3 (‘Iroquois related homeobox 2’ (IRX2), GCG, IRX1, ARX), whereas only two genes showed a bivalent histone modification profile (DPP4, PTPRD) (FIG. 5B). As expected, most α-cell-specific genes were marked only by H3K4me3 in α-cells. However, the genes PTPRD, IRX1, and the locus encoding the α-cell specific transcription factor ARX were marked bivalently (FIG. 5B). Most β-cell specific genes important for β-cell function displayed monovalent H3K4me3 enrichment (with the exception of HDAC9) in β-cells (FIG. 5C), while seven of twelve β-cell specific loci were marked bivalently in α-cells, including the functionally relevant genes PCSK1 and GLP1R and the genes encoding the crucial β-cell specific transcription factors MAFA and PDX1. These findings were extended by utilizing previously published H3K4me3 and H3K27me3 data of CD4-positive T-cells (23) and comparing the pancreatic histone modification profiles described herein to the histone modification landscape of this extra-pancreatic cell type. The histone modification profiles of all strongly α-cell, β-cell, and exocrine-specific genes in α-, β-, exocrine, and CD4+ T-cells can be found in Branswig et al. J. Clin. Invest. 2013 123(3):1275-84. Significant, monovalent enrichment for the activating H3K4me3 mark was found in only 4 of the 34 selected α-, β-, and exocrine-cell specific genes (See Table 8, below) as these pancreatic genes are not active in lymphoid cells. Therefore, α-cells preserve high bivalency in many genes known to be crucial for endocrine cell development and function.

The high incidence of bivalent marks in α-cells, the interesting bivalent pattern of β-cell and α-cell-specific transcription factors in α-cells, and the large overlap with bivalently-marked transcriptional regulators in hESC raised the likelihood that epigenomic manipulations could be exploited to reprogram human α-cells towards the β-cell phenotype. Several drugs, such as adenosine dialdehyde (Adox) and 3-Deazaneplanocin A (DZNep) interfere with histone methylation (24). The general histone methyltransferase inhibitor Adox, which amongst others decreases H3K27me3 levels (24), was employed to test whether modulation of the histone methylation status of human pancreatic islets could promote reprogramming. To validate the effectiveness of the histone methyltransferase inhibitor Adox in human islet tissue, the H3K27me3 modification landscape after Adox-treatment was investigated. The small numbers of cells recovered after Adox-treatment and FACS analysis did not allow cell-type specific analysis of the H3K27me3 profiles, so the H3K27me3 profiles of whole human islets cultured in the absence or presence of Adox were compared. This experiment allowed assessment of whether H3K27me3 levels of repressed genes are decreased after Adox-treatment.

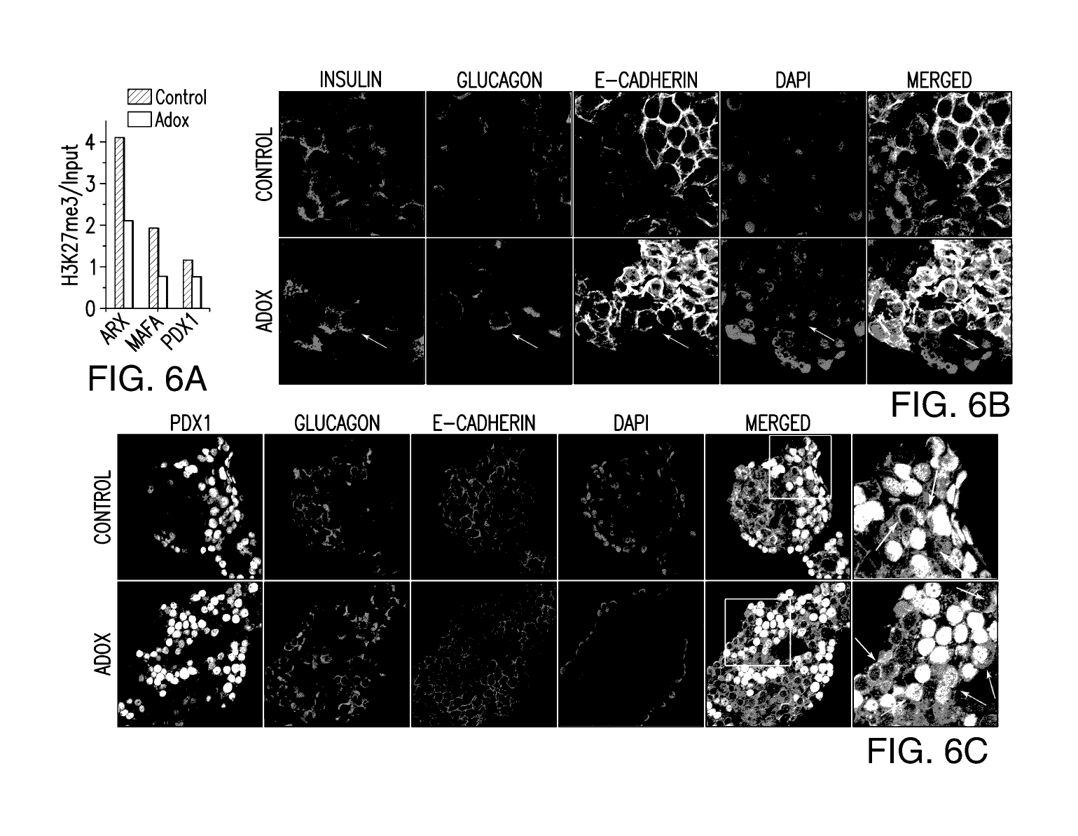

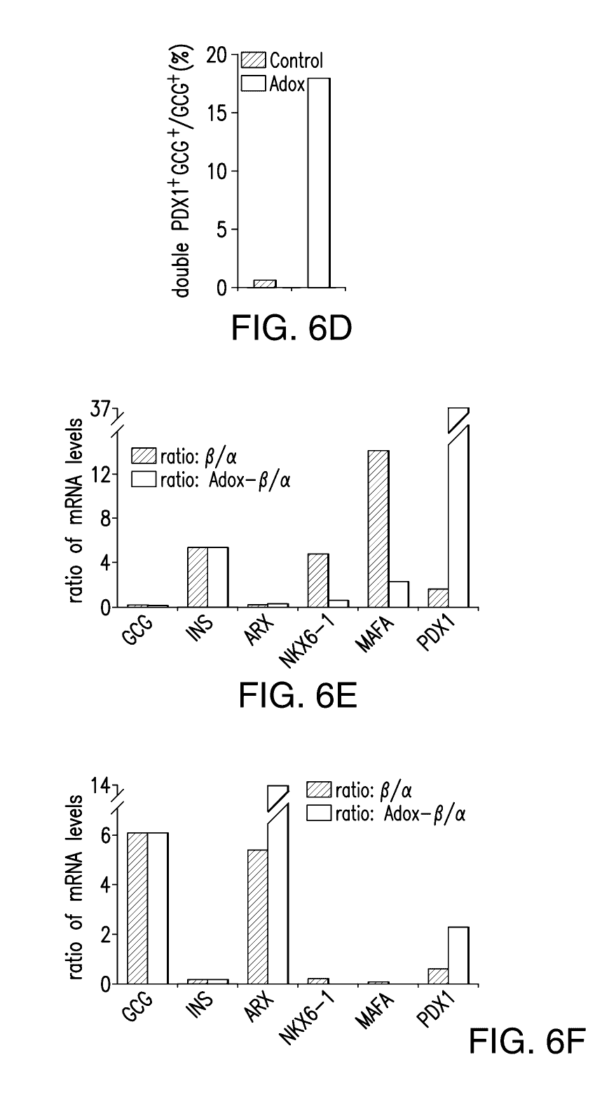

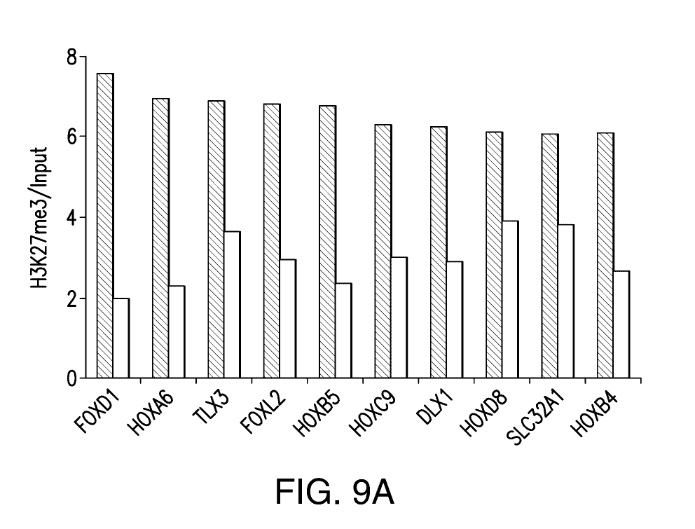

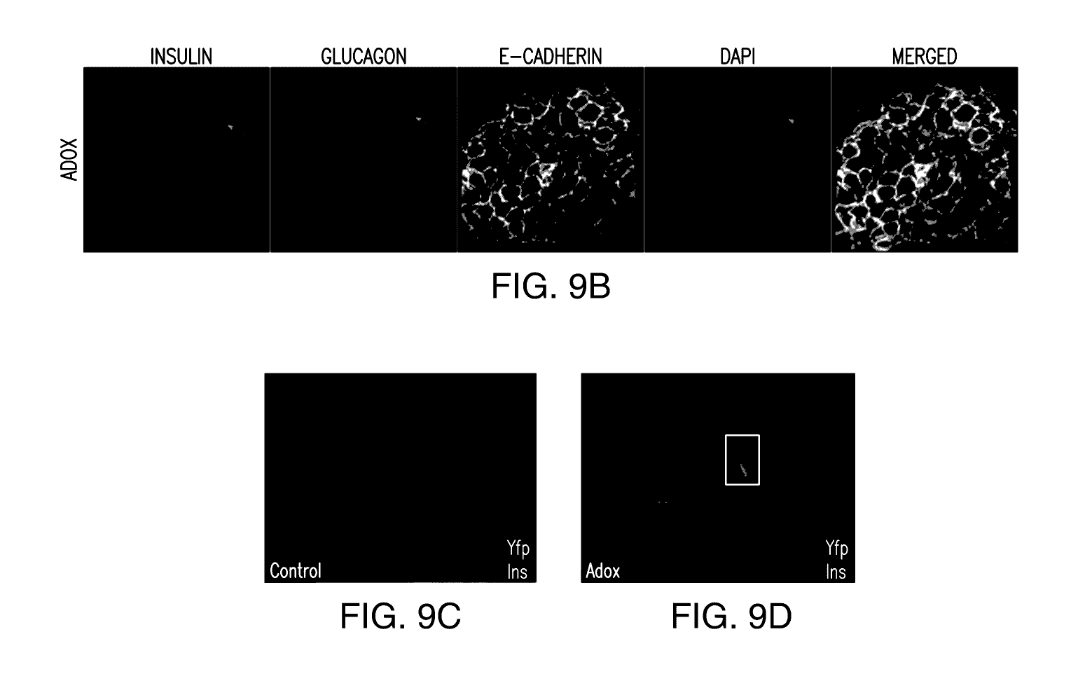

Indeed, analysis of genes that carry a bivalent mark or a monovalent H3K27me3 mark in all pancreatic cell populations displayed a strong decrease in H3K27me3 enrichment after Adox-treatment (FIG. 9A). In addition, this experiment approximated a cell-type-specific analysis of the H3K27me3 profiles of a subset of β-cell specific pancreatic transcription factors, namely those which are marked bivalently in α-cells, but monovalently by H3K4me3 in β-cells (MAFA and PDX1). Since α- and β-cells comprise the vast majority (approximately 90%) of the human islet (25) and exocrine cells do not survive in culture (26), any change in H3K27me3 enrichment levels at these loci is thus indicative of changes in α-cells at the MAFA and PDX1 loci, as there is no H3K27me3 present at these genes in β-cells to begin with. In addition, the H3K27me3 profile of the α-cell specific transcription factor ARX, which is marked bivalently in α-cells, but monovalently by H3K27me3 in β-cells, was investigated. The expected decrease of H3K27me3 enrichment at these three gene loci after Adox-treatment was confirmed by H3K27me3 ChIP-Seq analysis (FIG. 6A).

Treatment of human islets with Adox resulted in the occasional co-occurrence of glucagon and insulin granules within the same islet cell, which was not observed in untreated islets (FIG. 6B, FIG. 9B). A priori, the co-localization of glucagon with insulin could be due to α- to β-cell fate or β- to α-cell fate conversion. As lineage tracing is not possible in human samples, a murine genetic lineage tracing model was employed to assess the origin of the dual hormone positive cells. For this purpose, islets from GlucagonCre; Rosa26EYFP mice were treated, in which α-cells are permanently marked by yellow fluorescence protein (YFP) expression (27, 28) with Adox in vitro. Adox-treated islets showed insulin granules in YFP+ cells, which were not observed in untreated control islets (Supplemental FIGS. 3, C and D), supporting partial conversion of α-cells to the β-cell fate. Unfortunately, at the present time no inducible Glucagon-Cre ER line exists, and the possibility that Adox-treated β-cells activated the Glucagon-Cre promoter cannot be excluded. Since the co-localization of glucagon and insulin granules was observed in a small number of cells, further investigation was directed at whether the important β-cell marker PDX1 was present in glucagon-positive cells, indicating the initiation of partial reprogramming events. Strikingly, it was found that Adox-treatment caused nuclear PDX1-expression in many glucagon-positive cells (FIGS. 6, C and D).

TABLE 8
Histone modification profiles of selected α-, β-, and exocrine-specific
genes in α-, β-, exocrine and CD4+ T-Cells.

| Gene | Alpha | Beta | Exocrine | T-Cells |
|---|---|---|---|---|
| HNF1A | H3K4me3 | H3K4me3 | H3K4me3 | — |
| PCSK2 | H3K4me3 | H3K4me3 | H3K27me3 | H3K27me3 |
| IRX2 | H3K4me3 | H3K27me3 | H3K27me3 | H3K27me3 |
| GCG | H3K4me3 | H3K27me3 | H3K27me3 | H3K27me3 |
| DPP4 | H3K4me3 | Bivalent | Bivalent | H3K4me3 |
| PTPRD | Bivalent | Bivalent | H3K27me3 | H3K27me3 |
| ARX | Bivalent | H3K27me3 | H3K27me3 | H3K27me3 |
| IRX1 | Bivalent | H3K27me3 | H3K27me3 | H3K27me3 |
| MAFA | Bivalent | H3K4me3 | H3K27me3 | H3K27me3 |
| PCSK1 | Bivalent | H3K4me3 | H3K27me3 | H3K27me3 |
| KCNQ2 | Bivalent | H3K4me3 | H3K27me3 | H3K27me3 |
| IAPP | Bivalent | H3K4me3 | H3K27me3 | — |
| GLP1R | Bivalent | H3K4me3 | H3K4me3 | H3K27me3 |
| PDX1 | Bivalent | H3K4me3 | H3K4me3 | Bivalent |
| HDAC9 | Bivalent | Bivalent | H3K27me3 | H3K4me3 |
| INS | H3K27me3 | H3K4me3 | H3K27me3 | — |
| INS-IGF2 | H3K27me3 | H3K4me3 | H3K27me3 | H3K27me3 |
| CDKN1C | H3K4me3 | H3K4me3 | H3K4me3 | H3K4me3 |
| KCNJ11 | H3K4me3 | H3K4me3 | H3K4me3 | H3K4me3 |
| SLC30A8 | H3K4me3 | H3K4me3 | H3K27me3 | H3K27me3 |
| NKX6-1 | H3K4me3 | H3K4me3 | Bivalent | H3K27me3 |
| AMY1A | — | — | — | — |
| AMY1B | — | — | — | — |
| AMY1C | — | — | — | — |
| AMY2A | H3K27me3 | H3K27me3 | — | — |
| AMY2B | H3K27me3 | H3K27me3 | H3K27me3 | — |
| PNLIP | H3K27me3 | H3K27me3 | — | H3K27me3 |
| CTRB1 | — | — | H3K4me3 | — |
| CTRB2 | — | — | H3K4me3 | — |
| SPINK1 | — | H3K27me3 | H3K4me3 | H3K27me3 |
| PRSS3 | H3K4me3 | Bivalent | — | H3K27me3 |
| JAG1 | H3K4me3 | Bivalent | H3K4me3 | Bivalent |
| SOX9 | Bivalent | Bivalent | H3K4me3 | H3K27me3 |
| PTF1A | Bivalent | H3K27me3 | H3K4me3 | H3K27me3 |

To investigate the transcriptional changes in Adox-treated human islets and confirm increased PDX1 expression, FACS analysis after Adox-treatment was performed using the same antibody panel as described above, followed by RNA-Seq analysis (n=2 treated α, n=2 treated β, technical replicates) and focused on cell-type-specific hormones and transcription factors. To assess whether Adox-treated β-cells become more α-cell like or vice versa, the ratio of expression levels in untreated β-cells and untreated α-cells (β/α) were first compared to the ratio in Adox-treated β-cells and untreated α-cells (Adox-β/α) (FIG. 6E). A decrease in NKX6-1 and MAFA levels, an increase in PDX1 levels and no change in insulin expression were found and no change in the expression levels of the α-cell specific genes glucagon or ARX were observed, giving no indication for a gain of α-cell identity. Second, the ratio of expression values in untreated α-cells and untreated β-cells (α/β) were compared to the ratio in Adox-treated α-cells and untreated β-cells (Adox-α/β) to elucidate whether Adox-treated α-cells become more ‘β-cell like’ (FIG. 6F). This analysis revealed no change in insulin and glucagon levels and an increase in ARX levels. Interestingly, a slight decrease in NKX6-1 and MAFA levels and an increase in PDX1 expression were detected. Taken together, these results confirm the observations of PDX1 expression in glucagon-positive cells by immunofluorescent staining and favor partial α- to β-cell fate conversion over the alternative.

### Discussion

Epigenetic modifications play important roles in the differentiation and functional maintenance of cell-types and contribute to the development of complex diseases (29). Therefore, it is crucial to determine the specific epigenomic and transcriptional landscape of disease-relevant cell-types and understand the epigenetic mechanisms involved in maintaining or converting their cell-type specific identity. Previous studies have shown that reprogramming of α-towards the β-cell fate can occur under extreme conditions in mice (5-7). As described herein, using ChIP-Seq and RNA-Seq analysis, the cell-type-specific, genome-wide H3K4me3 and H3K27me3 profiles and transcriptional landscapes of human α- and β-cells were investigated and the cell-type specific differences were determined, and these results show that there is an epigenomic basis for this reprogramming potential.

The transcriptome analysis revealed clusters of α-, β- and exocrine-specific genes, and determined genes that are associated with an increased risk for non-autoimmune forms of diabetes in genome-wide association studies among α- and β-cell specific genes, which stresses the relevance of α- and β-cells in diabetes development. HDAC9 is also among the human β-cell specific genes (FIG. 2B). Previous studies observed β-cell specific expression of the histone deacetylase 9 (Hdac9) in mice and detected an increase in β-cell mass in Hdac9−/− mice (4). HDAC-inhibitors, more specifically HDAC9-inhibitors, might show a similar effect in human islets, and if so, HDAC9 is a target for diabetes treatment.

This Example shows that human α-, β-, and exocrine cells share very similar monovalent H3K4me3 and H3K27me3 histone modification maps. This does not support previously published murine H3K4me3 modification profiles, in which β-cells clustered with neural tissues, but not acinar cells (30). This discrepancy could be due to the different experimental setup. While van Arensbergen and colleagues investigated the H3K4me3 profiles using promoter arrays with limited coverage and sensitivity, genome-wide H3K4me3 ChIP-Seq analysis was performed in this study resulting in the analysis of a larger number of genes.

The results discussed herein show that pancreatic α-cells carry hundreds of bivalent marks on developmental regulatory genes, and display a bivalent modification profile remarkably similar to hESC, indicating a more plastic epigenomic state for α-cells than for β- and exocrine cells. Importantly, many β-cell specific signature genes involved in gene regulation are bivalently marked in α-cells. Therefore, these results indicate that this plastic epigenomic state of the α-cell explains, in part, the relative ease in which α-cells have been reprogrammed towards to β-cell fate in various mouse models (5-7). In contrast, exocrine cells display the smallest number of bivalent marks (FIG. 4A), which could explain the necessity of enforced expression of multiple key transcription factors to achieve limited conversion of exocrine cells towards the β-cell fate in vivo (31).

Remarkably, the simple treatment of islets with the unspecific histone methyltransferase inhibitor Adox results in partial reprogramming of endocrine cell fates. After Adox-treatment, co-localization of insulin and glucagon in a small number of cells was observed, but co-expression of the β-cell marker PDX1 and glucagon was observed at a much higher frequency. The discrepancy between the low number of GCG+/INS+ and the much higher number of GCG+/PDX1+ cells indicates that reprogramming was initiated in a substantial sub-population of α-cells, but was nearly completed in only a few. The explanation for this incomplete reprogramming might be provided in the RNA-Seq analysis of Adox-treated α- and β-cells. Although the co-localization of INS or PDX1 and GCG could, a priori, be due to 13- to α-cell or α- to β-cell fate conversion, there are multiple lines of evidence indicating that it is caused by partial reprogramming of α- to β-cell fate: (1) the higher genomewide bivalency of α-cells, especially at developmental regulatory genes and transcriptional regulatory genes expressed specifically in β-cells, (2) the high overlap of bivalently marked transcriptional regulators of α-cells with hESC, (3) the fact that two of the three β-cell specific transcription factors are marked bivalently (PDX1 and MAFA) in α-cells, (4) the co-localization of insulin in lineage-labeled α-cells of GlucagonCre; Rosa26EYFP mice, and (5) the expression patterns in Adox-treated α- and β-cells, which is discussed in more detail below.

The conducted expression analysis of α- and β-cell specific hormones and transcription factors following Adox-treatment supports partial α- to β-cell conversion, provides evidence for the limited specificity of Adox, and explains the incomplete conversion of the α- to the β-cell fate. For example, although NKX6-1 is monovalently marked by H3K4me3 in α- and β-cells and therefore should not be affected by Adox-treatment, its expression is decreased in Adox-treated α- and β-cells. This is likely due to the unspecific nature of the histone methyltransferase inhibitors currently available (24). In addition, MAFA, a bivalently marked gene in α-cells, is not induced and maintains extremely low mRNA levels after Adox-treatment. The persistently low expression of NKX6-1 and MAFA, and possibly the increased expression of ARX in Adox-treated α-cells, is likely contributing to the incomplete reprogramming of α- to β-cells. In fact, Zhou and colleagues showed that enforced expression of Neurog3, Pdx1, and Mafa is necessary to achieve reprogramming of adult exocrine cells to P3-like cells in mice (31), which stresses the requirement of Mafa expression for the accrual of partial β-cell identity. Importantly, the consistently low ARX expression in Adox-treated β-cells, and the increased PDX1 expression in treated α-cells, supports the switch from α- to β-cell fate, and not vice versa. Given the limited specificity of currently available ‘epigenomic drugs’, and the absence of cell division in cultured islets, the fate conversion achieved was only partial. Nevertheless, these findings indicate that a more targeted manipulation of the histone methylation signature in a histone modification- and gene specific manner can be exploited to promote a more complete human α- to β-cell fate conversion.

The cell-type specific study of histone modification signatures and corresponding RNA-Seq based transcriptomes and the results described herein establish human cell-type-specific epigenomic plasticity, and indicate that epigenomic manipulation of fully differentiated human islet cells provides a path to cell reprogramming, which can be exploited for in vivo and in vitro differentiation protocols towards the β-cell fate and cell replacement-based therapy for diabetes.

### Example 2

### Targeting Differential DNA Methylation to “Rejuvenate” Old B-Cells

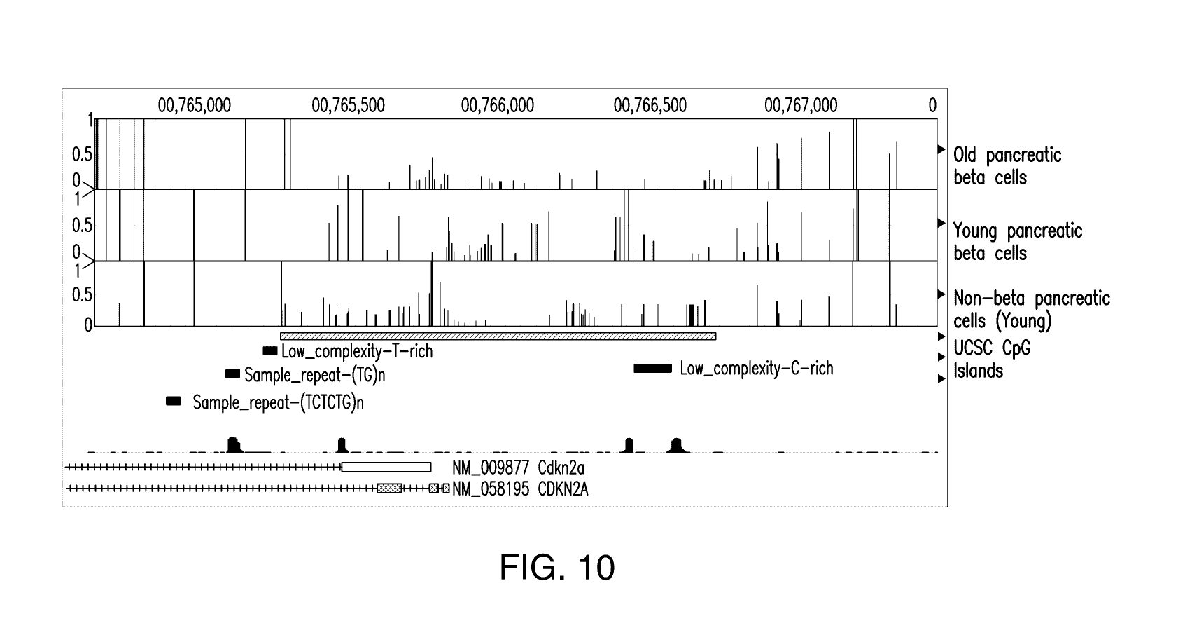

In this Example, epigenetic differences between embryonic stem cells and β-cells, at various developmental time points, were analyzed. Highly purified β-cell populations were sorted from seven day old, six week old, and twenty months old mice, representing β-cells with high, medium and near zero replication rates. Determination of the DNA methylome at base-resolution using whole genome was carried out using shotgun bisulite sequencing. Bisulfite converts all cytosines in the genome to uracil, which is read as T by DNA polymerase, with the exception of methylated cytosines. The methylome of the 3-cell differs dramatically from that of embryonic stem cells. In particular, multiple CpGs near the promoter of the p16/ARF locus, a locus known to be regulated in aging (Chen et al., 2009; Dhawan et al., 2011; Krishnamurthy et al., 2006; Tschen et al., 2009), are highly methylated in young β-cells, but become demethylated as the β-cell ages, indicative of reactivation of the locus (FIG. 10).

Integration of this methylome data with the histone marks at the three time points, allows the identification of the β-cell enhancers and promoters that are undergoing epigenetic regulation. Additionally, integration of the base-resolution, genomic analysis of DNA methylation marks with the altered gene expression and histone modifications observed in young (proliferative) versus old (quiescent) β-cells is used in order to identify potential targets for reversal of the quiescent phenotype of aged β-cells. The precise location of these relevant marks from the mouse is mapped back to the human genome, using β-cells from adult human donors.

TALs (Transcription activator-like), engineered to specific promoters and enhancers in human β-cells, is used to target DNA methylation (using DNMT3), demethylation (using TET enzymes), and histone H3K27me3 demethylation (using JMJD3). TALs are designed to uniquely and specifically recognize any 24 bp sequence in the human genome, and can be tethered to the catalytic domains of enzymes, such as transcriptional repressors or nucleases. Selected loci are epigenetically activated or repressed in human β-cells by fusing the catalytic domains of DNMT3, TET, or JMJD3 to specific TALs and transducing them into islets using lentiviral vectors, and the consequences on β-cell proliferation and function is assessed.

### Example 3

### Exploitation of the Epigenetic Plasticity of α-Cells for their Targeted Reprogramming into Functional β-Cells

As described above in Example 1, a general histone methyltransferase inhibitor was sufficient to activate insulin gene expression in a subset of human and mouse α-cells to promote partial conversion of α-cells to the β-cell fate.

A chemical screen of histone methylation inhibitors, including new H3K27me3-specific inhibitors, is performed to identify optimal compounds and conditions for the reprogramming of α-cells to β-cells. Glucagon-CreER, RosaYFP mice are used to confirm reprogramming of α- to β-cells by unequivocal genetic lineage tracing. Glucagon-CreER mice are created by developing a new Glucagon-CreER BAC transgenic line. Islets from tamoxifen-treated Glucagon-CreER, RosaYFP mice are exposed to histone methylation inhibitors and co-stained for insulin and YFP is performed to assess α- to β-cell reprogramming.

Additionally, Ezh2loxP/loxP, RosaYFP, Glucagon-CreER mice are generated to determine if polycomb-mediated repression via conditional gene ablation of the histone methyltransferase Ezh2 is sufficient to reprogram α-cells to β-cells. After tamoxifen treatment and activation of Cre in adult α-cells, these cells lose the repressive H3K27me3 mark and activate a bank of poised β-cell genes, causing cell fate conversion. This cell fate conversion is traced on the basis of RosaYFP expression. A novel single cell calcium imaging technology is employed to assess whether the reprogrammed β-cells have taken on the glucose responsiveness of true β-cells.

TAL effectors fused to the catalytic domain of the histone H3K27me3 demethylase JMJD3 targeted to the Pdx1 and MafA genes and additional regulatory genes that are bivalently marked in α-cells, but monovalently marked by HeK4me3 in β-cells, are generated. These targeted epigenetic modifiers are delivered to human and mouse islets using lentiviral vectors to stimulate reprogramming. These TAL-demethylases are delivered to α-cells using aptamers, to carry out ‘epigenomic cell therapy’ for diabetes.

### Example 4

### Targeting Differential DNA Methylation to “Rejuvenate” Old B-Cells

Aging produces dramatic changes in the epigenome of the β-cell. These epigenetic changes can be targeted to restore proliferative potential to mature β-cells. Studies on the p16 locus (Chen et al., 2009; Dhawan et al., 2011; Krishnamurthy et al., 2006; Tschen et al., 2009), indicate a change in the DNA methylation status as the β-cell ages and loses its replicative potential. All the relevant loci that undergo epigenetic changes in old versus young β-cells are mapped and a TAL-based approach is employed to specifically target these epigenetic changes, in an attempt to “epigenetically rejuvenate” old β-cells.

Integration of the base-resolution, genomic analysis of DNA methylation marks with altered gene expression (from RNAseq) and histone modifications (H3K4me3 and H3K27me3) observed in β-cells sorted from seven day old, six week old, and twenty months old MIP-GFP mice will allow the identification of potential target genes for reversal of the quiescent phenotype of old β-cells. The precise location of these relevant methylation and histone marks to the human genome is mapped back using β-cells from adult human donors (from the Integrated Islet Distribution Program). Sorting human β-cells is performed using the cell surface antibody panel previously described (Bramswig et al., 2012; Dorrell et al., 2011).

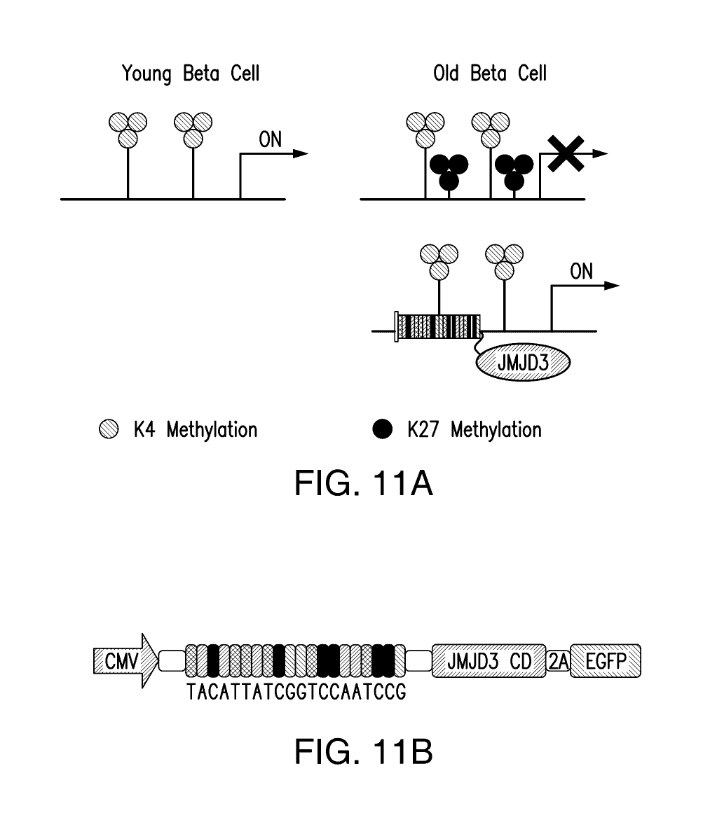

DNA methylation (using DNMT3), demethylation (by initial hydroxy-methylation using TET enzymes), and histone H3K27me3 demethylation (JMJD3) to the relevant loci is targeted using TALs (Transcription activator-like) engineered to specific promoters and enhancers in human β-cells. TALs can be designed to uniquely and specifically recognize any 24 bp sequence in the human genome, and can be tethered to the catalytic domains of enzymes, such as transcriptional repressors or nucleases (Boch, 2011; Scholze and Boch, 2011). Select loci is epigenetically activated or repressed in human β-cells, and the consequences on β-cell proliferation and function are determined. This is performed by fusing the catalytic domains of DNMT3, TET or JMJD3 to specific TALs targeting the cis-regulatory elements of a gene of interest, and transducing them into human islets using lentiviral vectors. Lentiviral vectors have been used for the transduction of human islets previously, with more than 50% infection efficiency for β-cells. The general approach is outlined in FIG. 11 for the case of the JMJD3 histone demethylase; analogous methods are used for DNA methylation and demethylation. All constructs can be bicistronic, allowing for the expression of the TAL together with eGFP, enabling us to stain or sort the transduced cell after completion of the experiment.

The efficacy of the epigenetic targeting of the TALs is analyzed by FACS sorting of the transduced islet cells, isolation of chromatin, and determination of the relevant epigenetic marks. For H3K27me3 and 5hmC, the initial product of the TET enzyme, ChIP followed by qPCR is employed. For DNA methylation, bisulfite sequencing of the relevant loci is employed.

Lentivirally-transduced human islets are assessed for their β-cell proliferation rate by in vitro culture in BrdU-containing medium and subsequent immunofluorescent detection of BrdU and eGFP, and after transplantation of human islets into immunodeficient NRG Akita-diabetic mice (Brehm et al., 2010). Islets are implanted under the kidney capsule and the grafts recovered after four weeks. Mice are given BrdU in the drinking water to capture all DNA-synthesis events. Recovered grafts are immunostained for β-cell markers, BrdU and eGFP to assess DNA replication, and also for γ-H2AX to exclude DNA synthesis as result of the DNA damage response, which we have found to be an essential control (Rieck et al., 2012).

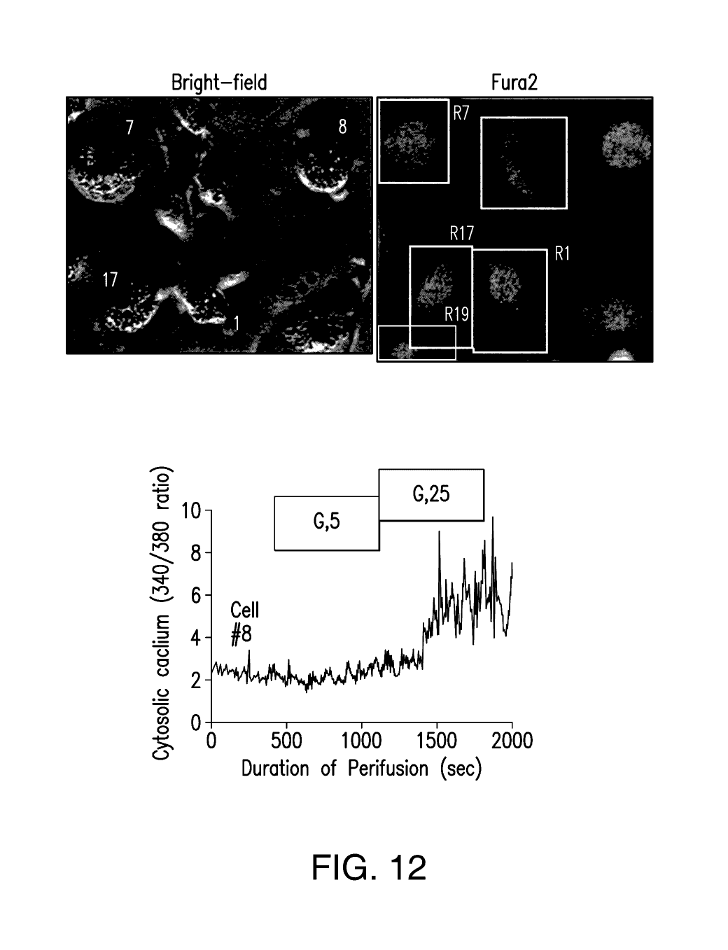

β-cells are tested for functionality and glucose responsive by determining intracellular calcium levels, which are the ultimate trigger of insulin granule fusion with the plasma membrane, in response to different glucose levels (see FIG. 12). The number of new-born β-cells that have maintained normal glucose responsiveness and thus function is determined by fixing the cover slip with the single islet cells post calcium imaging, and immunostaining them for BrdU and insulin. This allows direct comparison of the glucose responsiveness of old versus new β-cells.

### Example 5

### Exploitation of the Epigenetic Plasticity of A-Cells for their Targeted Reprogramming into Functional B-Cells

Recent experiments in transgenic mice have shown developmental plasticity of pancreatic α-cells, at least under extreme condition of total β-cell loss or over-expression of transcriptional master regulators (Collombat et al., 2009; Thorel et al., 2010; Yang et al., 2011). This plasticity is due, in part, to the retained bivalent state of thousands of genes in α-cells. Additionally, epigenetic drugs can be used for partial α- to β-cell fate conversion (Bramswig et al., 2012). Therefore, epigenetic manipulations, both pharmacologic and genetic, can be exploited for α- to β-cell reprogramming.

A chemical screen of histone methylation inhibitors, including new H3K27me3-specific inhibitors developed by GSK, is performed to identify optimal compounds and conditions for the reprogramming of α-cells to β-cells. Additionally, given the success with the first trial of a histone methyltransferase inhibitor (Adox), which was employed at one concentration and time point (see Example 1), the efficacy of this pharmacological intervention will be optimized using standard dose-response and time course experiments. Drugs tested are Adox, DZnep (another histone methyltransferase inhibitor), both alone and in combination with 3,5-disubstituted isoxazoles (Isx), the likely histone acetyl transferase activator recently shown to induce differentiation and increase insulin production in β-cells (Dioum et al., 2011). Pancreatic islets from GlucagonCre; Rosa26eYFP mice or Glucagon-CreER, Rosa26eYFP mice (once available, see Subaim 2.2), are co-stained for insulin and YFP after incubation with the drugs.

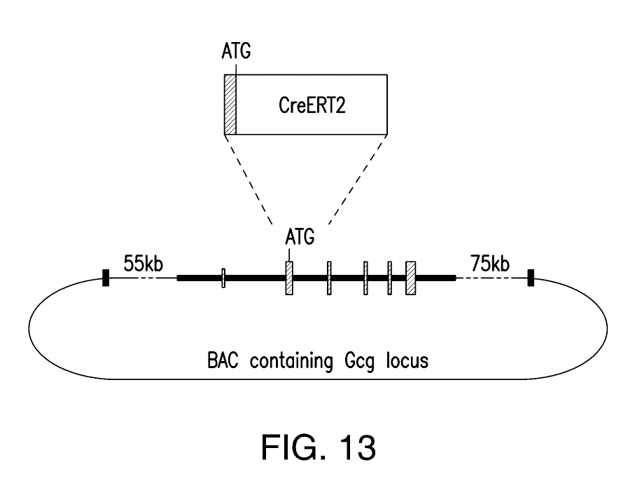

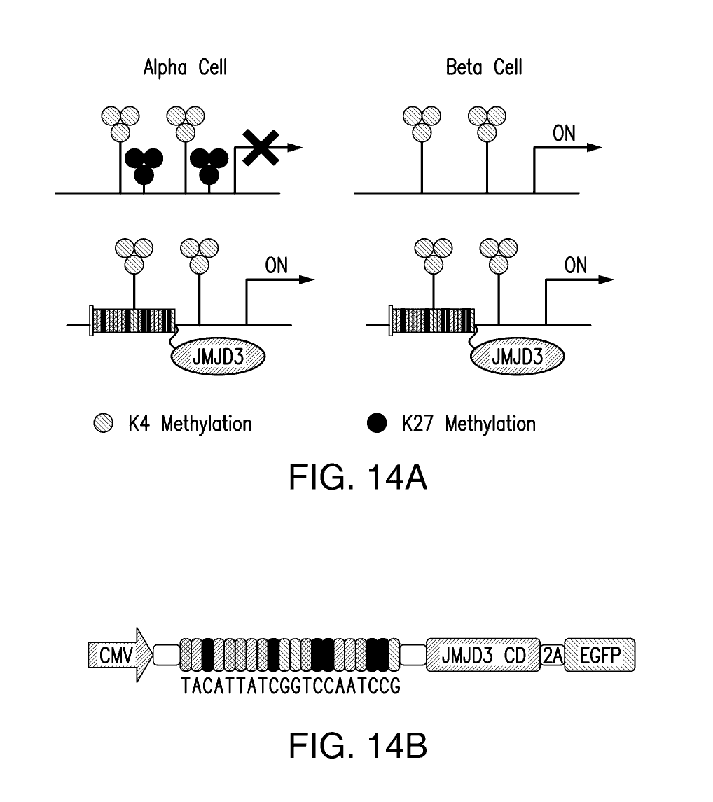

Glucagon-CreER, Rosa26eYFP mice are used to confirm reprogramming of α- to β-cells by unequivocal genetic lineage tracing. An inducible GlucagonCreER transgenic line is created, in which α-cells can be permanently labeled by tamoxifen-treatment prior to treatment with Adox or similar compounds. A BAC transgene with all upstream (55 kb) and downstream (75 kb) flanking regions of the preproglucagon gene is used (see FIG. 13). BAC DNA will be modified as outlined in FIG. 14 by BAC recombineering. Transgenic mice will be obtained after pronuclear injection of purified, modified BAC DNA, and transgenic offspring are identified by PCR of tail snip DNA. Transgenic lines are established and tested by breeding to RosaYFP mice. Glucagon-CreER, Rosa26eYFP mice are treated with tamoxifen either by intraperitoneal (i.p.) injection or by subcutaneous implantation of a slow-release tamoxifen pellet (Gao et al., 2007), and efficiency and specificity of CreER mediated target ablation are determined by dual-label immunofluorescence staining for YFP and glucagon and YFP and insulin, respectively. The best line, in terms of low background activity and high, α-cell specific induced gene ablation, are selected for further studies. Islets are isolated from tamoxifen-treated Glucagon-CreER, Rosa26eYFP mice and treated with Adox, or a combination of compounds, to evaluate if these agents induce true cell fate conversion of α-cells to β-cells.

Ezh2loxP/loxP, Rosa26eYFP, Glucagon-CreER mice are generated to assess whether removal of polycomb-mediated repression via conditional gene ablation of the histone methyl transferase Ezh2 is sufficient to reprogram α-cells to β-cells. After tamoxifen treatment and activation of Cre in adult α-cells following tamoxifen treatment, these cells lose the repressive H3K27me3 mark and activate a bank of poised β-cell genes, causing cell fate conversion, which can be traced on the basis of RosaYFP expression.

Cell fate conversion from α-cells to β-cells as consequence of Ezh2 ablation in α-cells is assessed by dual-label immunofluorescence staining for YFP and insulin as described above. In order to assess whether the reprogrammed β-cells have taken on the glucose responsiveness of true β-cells, single cell calcium imaging technology as described above is employed. Islets are collected from tamoxifen-treated Ezh2loxP/loxP, Rosa26eYFP, Glucagon-CreER mice, and YFP+ cells sorted by FACS and plated on cover-slips for calcium imaging in response to glucose. After recording of the calcium-response, the cells are fixed and immunostained for insulin and YFP. Alignment of the images with the calcium data allows determination if reprogrammed former α-cells show the glucose-response behavior of normal j-cells. Sorted, reprogrammed cells are employed for genome-wide analysis of H3K27me3 marks by ChIPSeq, to determine at which loci the histone modification pattern was altered during the conversion from α- to β-cell.

TAL effectors fused to the catalytic domain of the histone H3K27me3 demethylase JMJD3 targeted to the Pdx1 and MafA genes and additional relevant regulatory genes that are shown to be bivalently marked in α-cells are generated (Bramswig et al., 2012). These targeted epigenetic modifiers to human and mouse islets are delivered using lentiviral vectors in order to stimulate reprogramming. The general strategy is outlined in FIG. 14. All constructs can be bicistronic, allowing the expression of the TAL together with eGFP, thereby allowing the staining or sorting of the transduced cell at the end of the experiment for further analyses. Separate constructs are designed to target the relevant cis-regulatory elements in human and mouse cells. Human and mouse islets (from Glucagon-CreER, Rosa26eYFP mice) are transduced with one or more lentiviral vectors targeting TAL-JMJD3 to the relevant cis-regulatory elements. After in vitro culture for up to one week, islets are harvested and analyzed for insulin/glucagon co-expressing cells in case of human islets, and for insulin/YFP co-expression in case of mouse islets. The percentage of re-programmed cells are determined, and the degree of cell fate conversion to proper glucose-responsiveness assays by single cell calcium imaging are performed as outlined above. Transduced cells are sorted based on the eGFP co-expression of the successfully infected cells, and the extent of epigenetic modification of the relevant loci assayed by ChIP-qPCR or ChIPSeq for H3K27me3. TAL-demethylases are delivered to α-cells, in a clinical setting, using aptamers in order to carry out ‘epigenomic cell therapy’ for diabetes.

### REFERENCES

1. Ashcroft, F. M., and Rorsman, P. 2012. Diabetes mellitus and the beta cell: the last ten years. Cell 148:1160-1171.
2. Unger, R. H., and Orci, L. 1975. The essential role of glucagon in the pathogenesis of diabetes mellitus. Lancet 1:14-16.
3. Haumaitre, C., Lenoir, O., and Scharfmann, R. 2008. Histone deacetylase inhibitors modify pancreatic cell fate determination and amplify endocrine progenitors. Molecular and cellular biology 28:6373-6383.
4. Lenoir, O., Flosseau, K., Ma, F. X., Blondeau, B., Mai, A., Bassel-Duby, R., Ravassard, P., Olson, E. N., Haumaitre, C., and Scharfmann, R. 2011. Specific Control of Pancreatic Endocrine {beta}- and {delta}-Cell Mass by Class IIa Histone Deacetylases HDAC4, HDAC5, and HDAC9. Diabetes 60:2861-2871.
5. Collombat, P., Xu, X., Ravassard, P., Sosa-Pineda, B., Dussaud, S., Billestrup, N., Madsen, O. D., Serup, P., Heimberg, H., and Mansouri, A. 2009. The ectopic expression of Pax4 in the mouse pancreas converts progenitor cells into alpha and subsequently 3-cells. Cell 138:449-462.
6. Thorel, F., Nepote, V., Avril, I., Kohno, K., Desgraz, R., Chera, S., and Herrera, P. L. 2010. Conversion of adult pancreatic alpha-cells to beta-cells after extreme beta-cell loss. Nature 464:1149-1154.
7. Yang, Y. P., Thorel, F., Boyer, D. F., Herrera, P. L., and Wright, C. V. 2011. Context-specific alpha-to-beta-cell reprogramming by forced Pdx1 expression. Genes & development 25:1680-1685.
8. Bhandare, R., Schug, J., Le Lay, J., Fox, A., Smirnova, O., Liu, C., Naji, A., and Kaestner, K. H. 2010. Genome-wide analysis of histone modifications in human pancreatic islets. Genome research 20:428-433.
9. Gaulton, K. J., Nammo, T., Pasquali, L., Simon, J. M., Giresi, P. G., Fogarty, M. P., Panhuis, T. M., Mieczkowski, P., Secchi, A., Bosco, D., et al. 2010. A map of open chromatin in human pancreatic islets. Nature genetics 42:255-259.
10. Stitzel, M. L., Sethupathy, P., Pearson, D. S., Chines, P. S., Song, L., Erdos, M. R., Welch, R., Parker, S. C., Boyle, A. P., Scott, L. J., et al. 2010. Global epigenomic analysis of primary human pancreatic islets provides insights into type 2 diabetes susceptibility loci. Cell metabolism 12:443-455.
11. Dorrell, C., Abraham, S. L., Lanxon-Cookson, K. M., Canaday, P. S., Streeter, P. R., and Grompe, M. 2008. Isolation of major pancreatic cell types and long-term culture-initiating cells using novel human surface markers. Stem cell research 1:183-194.
12. Bernstein, B. E., Mikkelsen, T. S., Xie, X., Kamal, M., Huebert, D. J., Cuff, J., Fry, B., Meissner, A., Wernig, M., Plath, K., et al. 2006. A bivalent chromatin structure marks key developmental genes in embryonic stem cells. Cell 125:315-326.
13. Mikkelsen, T. S., Ku, M., Jaffe, D. B., Issac, B., Lieberman, E., Giannoukos, G., Alvarez, P., Brockman, W., Kim, T. K., Koche, R. P., et al. 2007. Genome-wide maps of chromatin state in pluripotent and lineage-committed cells. Nature 448:553-560.
14. Lien, W. H., Guo, X., Polak, L., Lawton, L. N., Young, R. A., Zheng, D., and Fuchs, E. 2011. Genome-wide maps of histone modifications unwind in vivo chromatin states of the hair follicle lineage. Cell stem cell 9:219-232.
15. Guz, Y., Montminy, M. R., Stein, R., Leonard, J., Gamer, L. W., Wright, C. V., and Teitelman, G. 1995. Expression of murine STF-1, a putative insulin gene transcription factor, in β-cells of pancreas, duodenal epithelium and pancreatic exocrine and endocrine progenitors during ontogeny. Development 121:11-18.
16. Wu, K. L., Gannon, M., Peshavaria, M., Offield, M. F., Henderson, E., Ray, M., Marks, A., Gamer, L. W., Wright, C. V., and Stein, R. 1997. Hepatocyte nuclear factor 3beta is involved in pancreatic beta-cell-specific transcription of the pdx-1 gene. Molecular and cellular biology 17:6002-6013.
17. McCarthy, M. I. 2010. Genomics, type 2 diabetes, and obesity. N Engl J Med 363:2339-2350.
18. Ponting, C. P., Oliver, P. L., and Reik, W. 2009. Evolution and functions of long noncoding RNAs. Cell 136:629-641.
19. Moran, I., Akerman, I., van de Bunt, M., Xie, R., Benazra, M., Nammo, T., Arnes, L., Nakic, N., Garcia-Hurtado, J., Rodriguez-Segui, S., et al. 2012. Human beta Cell Transcriptome Analysis Uncovers lncRNAs That Are Tissue-Specific, Dynamically Regulated, and Abnormally Expressed in Type 2 Diabetes. Cell metabolism 16:435-448.
20. Huang da, W., Sherman, B. T., and Lempicki, R. A. 2009. Systematic and integrative analysis of large gene lists using DAVID bioinformatics resources. Nature protocols 4:44-57.
21. Huang da, W., Sherman, B. T., and Lempicki, R. A. 2009. Bioinformatics enrichment tools: paths toward the comprehensive functional analysis of large gene lists. Nucleic acids research 37:1-13.
22. Pan, G., Tian, S., Nie, J., Yang, C., Ruotti, V., Wei, H., Jonsdottir, G. A., Stewart, R., and Thomson, J. A. 2007. Whole-genome analysis of histone H3 lysine 4 and lysine 27 methylation in human embryonic stem cells. Cell stem cell 1:299-312.
23. Barski, A., Cuddapah, S., Cui, K., Roh, T. Y., Schones, D. E., Wang, Z., Wei, G., Chepelev, I., and Zhao, K. 2007. High-resolution profiling of histone methylations in the human genome. Cell 129:823-837.
24. Miranda, T. B., Cortez, C. C., Yoo, C. B., Liang, G., Abe, M., Kelly, T. K., Marquez, V. E., and Jones, P. A. 2009. DZNep is a global histone methylation inhibitor that reactivates developmental genes not silenced by DNA methylation. Molecular cancer therapeutics 8:1579-1588.
25. Brissova, M., Fowler, M. J., Nicholson, W. E., Chu, A., Hirshberg, B., Harlan, D. M., and Powers, A. C. 2005. Assessment of human pancreatic islet architecture and composition by laser scanning confocal microscopy. The journal of histochemistry and cytochemistry: official journal of the Histochemistry Society 53:1087-1097.
26. Campbell, I. L., Colman, P. G., and Harrison, L. C. 1985. Adult human pancreatic islet cells in tissue culture: function and immunoreactivity. J Clin Endocrinol Metab 61:681-685.
27. Herrera, P. L. 2000. Adult insulin- and glucagon-producing cells differentiate from two independent cell lineages. Development 127:2317-2322.
28. Srinivas, S., Watanabe, T., Lin, C. S., William, C. M., Tanabe, Y., Jessell, T. M., and Costantini, F. 2001. Cre reporter strains produced by targeted insertion of EYFP and ECFP into the ROSA26 locus. BMC developmental biology 1:4.
29. Petronis, A. 2010. Epigenetics as a unifying principle in the aetiology of complex traits and diseases. Nature 465:721-727.
30. van Arensbergen, J., Garcia-Hurtado, J., Moran, I., Maestro, M. A., Xu, X., Van de Casteele, M., Skoudy, A. L., Palassini, M., Heimberg, H., and Ferrer, J. 2010. Derepression of Polycomb targets during pancreatic organogenesis allows insulin producing beta-cells to adopt a neural gene activity program. Genome research 20:722-732.
31. Zhou, Q., Brown, J., Kanarek, A., Rajagopal, J., and Melton, D. A. 2008. In vivo reprogramming of adult pancreatic exocrine cells to beta-cells. Nature 455:627-632.
32. Tuteja, G., White, P., Schug, J., and Kaestner, K. H. 2009. Extracting transcription factor targets from ChIP-Seq data. Nucleic acids research 37:e113.
33. Lefterova, M. I., Steger, D. J., Zhuo, D., Qatanani, M., Mullican, S. E., Tuteja, G., Manduchi, E., Grant, G. R., and Lazar, M. A. 2010. Cell-specific determinants of peroxisome proliferator-activated receptor gamma function in adipocytes and macrophages. Molecular and cellular biology 30:2078-2089.
34. Malone, B. M., Tan, F., Bridges, S. M., and Peng, Z. 2011. Comparison of four ChIP-Seq analytical algorithms using rice endosperm H3K27 trimethylation profiling data. PLoS One 6:e25260.

### Example 6

### Tale-Mediated Epigenetic Supression of Cdkn2a (P16) Increases Replication in Primary Human Fibroblasts Introduction

Epigenetic modifications are a major determinant of gene expression programs, and inappropriate changes in these modifications can lead to a wide spectrum of diseases. Cancer is perhaps the most widely recognized disease area associated with aberrant epigenetic changes, and more recently epigenetic changes have been implicated in neurological, metabolic, and cardiovascular diseases (1). These modifications are known to be reversible, making them attractive drug targets. To date, clinicians have relied exclusively on general inhibitors of globally expressed epigenetic regulators, which are responsible for maintaining integrity of the entire genome (2). Thus, unintended effects of such epigenetic inhibitors may be particularly pervasive and deleterious. Therefore, there is a need for novel tools for interrogating specific epigenetic changes in the laboratory to enable novel therapeutic strategies.

DNA methylation has emerged as an important mechanism governing cellular reprogramming processes such as cell differentiation, cellular senescence, and disease. In mammalian cells, DNA methylation is most abundant on cytosine residues in the context of cytosine guanine dinucleotides, or CpG's, and when occurring at enhancers and promoters, is frequently associated with gene repression (3). DNA methylation patterns are established by the de novo DNA methyltransferases, DNMT3a and DNMT3b, and propagated across cell divisions by the maintenance DNA methyltransferase, DNMT1 (4,5).

In an experimental or therapeutic setting, targeted de novo DNA methylation may be accomplished by tethering the catalytic domain of a DNA methyltransferase (DNMT) to DNA binding proteins designed to bind specific gene loci, thereby affecting gene expression. Siddique and colleagues have pioneered this strategy by fusing DNMT catalytic subunits to an artificial zinc finger protein targeting the promoter of vascular endothelial cell growth factor A (VEGF-A) in a human cancer cell line, SOKV3 (6). However, challenges in designing artificial zinc fingers have limited the widespread use of this technology (7). Transcription activator-like effectors (TALEs) are a newer technology that is extremely modular, easy to assemble, and therefore a more efficient choice for targeted epigenome editing.

TALEs are DNA binding proteins endogenous to bacterial plant pathogens including the genus Xanthomonas. This class of proteins binds to specific regulatory regions in the host genome to modulate gene expression and promote bacteria survival. The central DNA binding domain of TALE proteins consists of a series of approximately 34-amino acid repeats, or monomers, which are polymorphic only at positions 12 and 13. These polymorphic residues, termed the repeat-variable-di-residue (RVD), determine DNA binding specificity, as each amino acid pair preferentially binds to one of the four nucleotides (8). Consequently, by assembling monomers in a particular order, TALEs can be engineered to bind specific DNA sequences.

Customized TALEs have been used to modulate transcription through conjugation to activator domains, such as VP64, and repressor domains, such as the mSin interaction domain (SID) (9,10). The potential for implementing TALEs to direct targeted epigenetic modifications has become increasingly recognized, as shown in recent studies targeting DNA and histone demethylation (11,12). However, this approach has not yet been used to repress gene expression by targeted DNA methylation. A key hurdle in widespread use of TALEs is that they are incompatible with lentivirus technology, a common approach in stable transmission of genes into host genomes, particularly with primary cells. The highly repetitive sequences of the TALE modules have a strong tendency to recombine (13), and we addressed this issue by reengineering the TALE repeat moieties to minimize direct DNA repeats without altering the coding sequence.

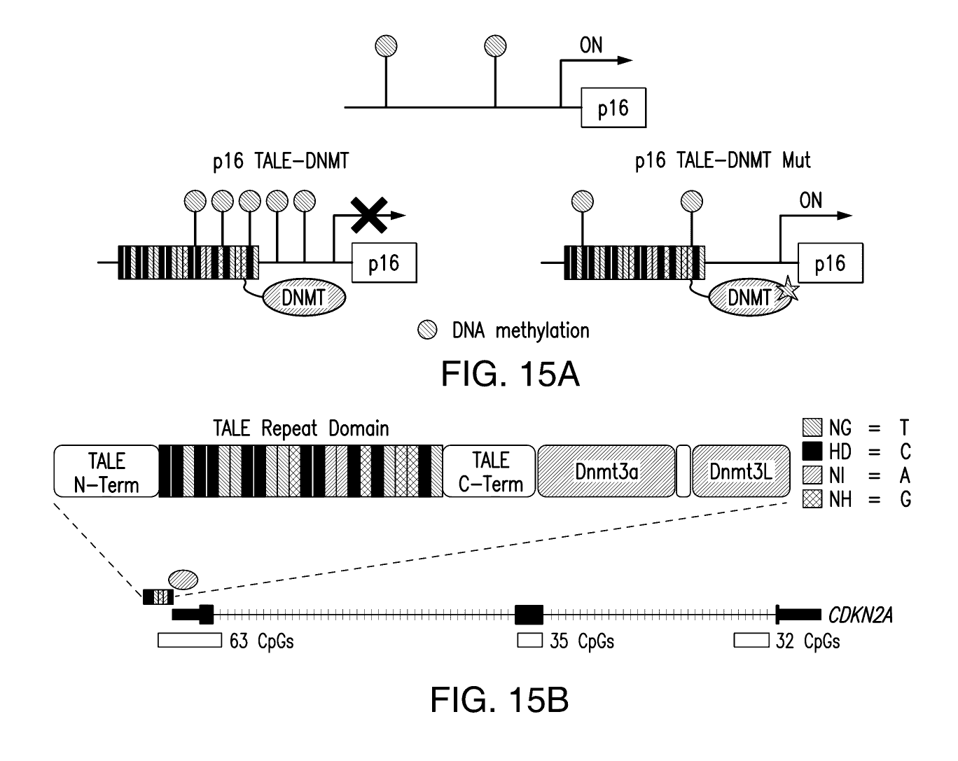

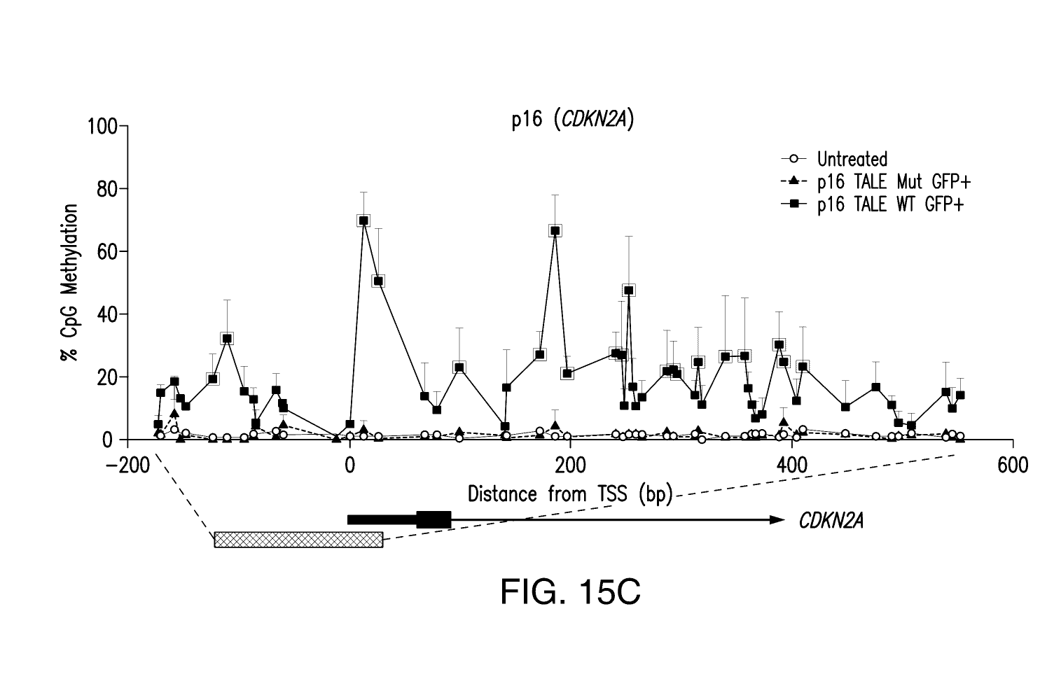

Our primary target for development of this novel “TALE-DNMT” strategy was the cell cycle inhibitor, p16, which is encoded by the CDKN2A gene (FIG. 15a). p16 is a universal regulator of cellular senescence, and CDKN2A was found to be the most common locus associated with age-related disease in a meta-analysis of GWAS conducted by Jeck and colleagues (14,15). Prior studies have shown that CDKN2A is regulated by DNA methylation, and decreasing p16 levels might aid in coaxing terminally differentiated cells back into the cell cycle, allowing for cell expansion for experimental or cell therapy uses (16,17).

Thus, we sought to target DNA methylation to the p16 (CDKN2A) locus using lentiviral delivery of TALE-DNMT fusion proteins to repress gene expression and thereby increase cellular proliferation. We also characterized the specificity of TALE-mediated epigenetic modifications, which to date has not been reported. Here, we show that epigenetic targeting of a single locus can indeed alter its gene expression and cellular functions without appreciable “off target” effects, thus demonstrating that the phenotype of primary human cells can be altered using epigenetic tools.

### Methods

TALE Target Selection and Construction

Twenty-four base pair TALE target sequences within the CDKN2A promoter were selected using the web-based tool, TAL Effector-Nucleotide Targeter (TALE-NT) 2.0, which optimizes unique binding sites within a specified target region (8,25). The sequence targeted in the present work was 5′-CCTCCTTCCTTGCCAACGCTGGCT-3′. Cloning vectors and TALE repeat monomer plasmids were obtained from the TALE Toolbox kit (Addgene). pTALETF vectors were modified to replace the VP64 domain with flag-tagged DNMT3α-3L by cloning. Following target selection, the 24 corresponding monomers were assembled into a modified TALE backbone containing either the DNMT3a-3L catalytic subunit or mutant DNMT3a-3L catalytic subunit through a series of golden gate digestion-ligation reactions, as described by Sanjana and colleagues (18). The mutant DNMT catalytic domain contained a catalytically inactive DNMT3a subunit due to a point mutation, E752A (19). The DNMT3α-3L DNA sequence was synthesized by Eurofins MWG Operon and cloned into the TALE backbone by restriction enzyme digestion followed by ligation. PCR primers including the E752A point mutation were used to amplify a DNA fragment from the TALE-DNMT plasmid containing the point mutation, and the fragment was subsequently cloned into the TALE-DNMT backbone by restriction enzyme digestion followed by ligation to generate the mutant construct. The point mutation is underlined in the reverse primer (PCR primers, Forward: 5′-CAAGCCCCAAGAAGAAGAGA-3′ and Reverse: 5′-CCCATGGCCACCACATTGGCAAAG AG-3′).

Jumbled TALE Lentivirus Design and Construction

Jumbled TALEs were designed by disrupting direct repeats greater than eleven nucleotides utilizing the degeneracy of the genetic code to change DNA sequence without altering protein coding sequence. In order to accommodate the size limitation of the lentiviral genome, the TALE target sequence was decreased to 18 bases instead of 24, a minimal CMV promoter was used, and eGFP was removed. The 18 base pair sequence targeted with the jumbled TALE was <5′-TCCTTGCCAACGCTGGCT-3′>. Series of plasmids containing jumbled sequences for 6 monomers were ordered from Eurofins MWG Operon with appropriate restriction sites such that these hexamers could be incorporated into the protocol described by Sanjana and colleagues at a Golden-Gate digestion-ligation step. TALE-DNMT constructs were then cloned into a lentiviral vector obtained from The Wistar Institute Protein Expression Core (Philadelphia, Pa.). Lentiviruses were also prepared by The Wistar Institute Protein Expression Core (Philadelphia, Pa.).

Transfection of HeLa Cells

HeLa cells (American Type Culture Collection) were seeded at a density of 5×10⁶ cells per 10 cm³. After 24 hours, cells were transfected with 12.5 μg of plasmid DNA using Lipofectamine® 2000 reagent (Life Technologies™) according to manufacturer's protocol. Forty-eight hours post-transfection, cells were trypsinized with 0.25% trypsin+EDTA and dispersed for FACS sorting for GFP. FACS sorting was performed by the Flow Cytometry and Cell Sorting Resource Laboratory (FCCSRL) at the University of Pennsylvania (Philadelphia, Pa.). Sorted populations were harvested using the Qiagen AllPrep DNA/RNA Mini Kit.

DNA Methylation Analysis

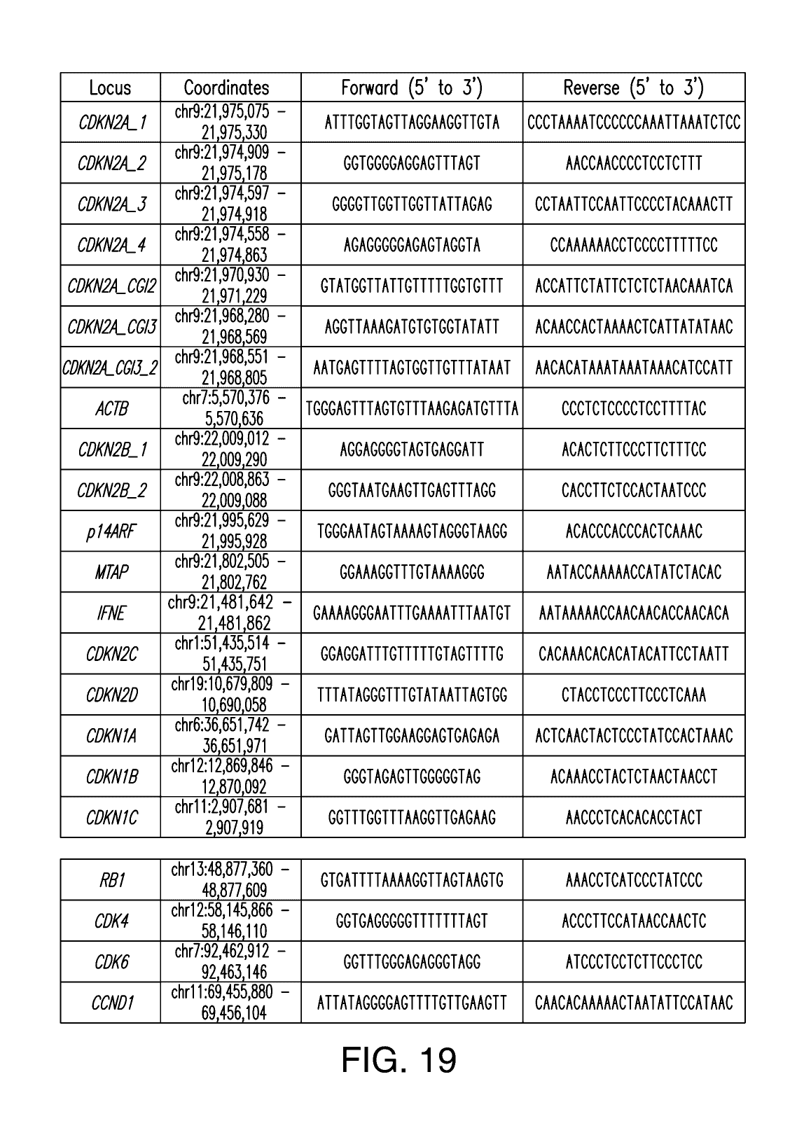

Genomic DNA was bisulfite-converted using the Qiagen EpiTect Bisulfite Kit (Qiagen GmbH) and target loci were PCR amplified using the PyroMark PCR Kit (Qiagen GmbH). Four primer pairs were designed to PCR amplify across the entire CDKN2A CpG island within the promoter region. Additional primer pairs were also designed to amplify regions within each control locus. Primers were designed to amplify approximately 250-300 base pair regions at the CpG island closest to the transcription start site of each gene. In instances when no CpG island was present, a sequence within the gene promoter was chosen. All primer sequences and genomic coordinates for each amplicon are listed in FIG. 19. Pyromark PCR reactions were carried out per manufacturer's instructions. DNA sequencing libraries were prepared with the automated Ovation® SP+ Ultralow DR Multiplex System (NuGEN Technologies Inc.) and subsequently sequenced on an Illumina MiSeq with 150 base pair paired-end reads. DNA sequences were aligned to an in silico bisulfite converted human genome using the BS Seeker program and analyzed by the Next Generation Sequencing Core at the University of Pennsylvania (Philadelphia, Pa.) (26). Only CpGs with sequence coverage greater than 1000 reads were considered. Average DNA methylation across regions was analyzed by one-way ANOVA with Tukey's correction for multiple comparisons when comparing more than two groups, and by a two-tailed t-test when comparing two groups. Individual CpGs were compared by multiple t-tests (P<0.05).

Quantitative Real-Time PCR for Gene Expression

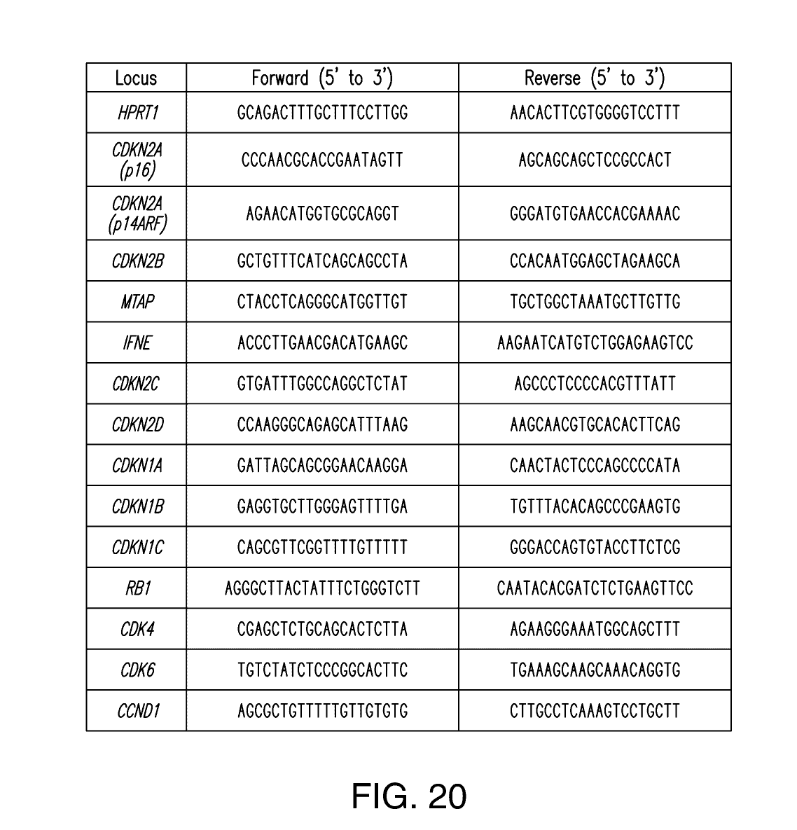

To assess mRNA levels, RNA extracted with the Qiagen AllPrep DNA/RNA Mini Kit was reverse-transcribed using SuperScript® II Reverse Transcriptase (Life Technologies™) to synthesize cDNA. qRT-PCR was performed on the Agilent Technologies Strategene Mx3000P using 2× Brilliant III SYBR® Green qPCR master mix plus ROX reference dye (Agilent Technologies). Thermal profiles were set according to manufacturer's protocol. mRNA levels were normalized to HPRT1. qPCR primer sequences are listed in FIG. 20. Differences in mRNA levels were compared by two-tailed t-tests (P<0.05).

PCR Assay

To demonstrate integration and transcription of full-length TALE DNA in lentiviral infections of HeLa, the TALE repeat moiety was amplified from genomic DNA and cDNA, respectively, with Herculase II Fusion DNA polymerase. (TALE repeat primers, Forward: 5′-CCAGTTGCTGAAGATCGCGAAGC-3′ and Reverse: 5′-TGCCACTCGATGTGATGTCCTC-3′). The Woodchuck hepatitis virus posttranscriptional regulatory element (WPRE) present in lentiviral constructs was used as a control (Forward: 5′-AGCGTCGACAATCAACCTCT-3′ and Reverse: 5′-GGCATTAAAGCAGCGTATCC-3′). PCR products were purified using the QIAquick® PCR Purification Kit (Qiagen GmbH), and 100 ng of each product was run on a 0.8% agarose gel.

HeLa Cell Lentivirus Transduction

HeLa cells were seeded at a density of 1×10⁶ cells per 10 cm³ in DMEM culture medium supplemented with 10% fetal bovine serum and penicillin/streptomycin. Twenty-four hours postseeding, cells were treated with 1×10⁷ titration units of lentivirus. Polybrene was added at a concentration of 8 ng/μl to enhance viral infection. Cells were harvested four days post-infection for either DNA/RNA extraction or preparation of protein lysate as described.

Western Blotting

Following transduction, cells were sedimented and homogenized in RIPA buffer. Cell lysates were sonicated, sedimented to remove cellular debris, and protein concentration was measured by the Millipore Direct Detect system. 50 μg of protein lysate was denatured with DTT and separated by size on a NuPAGE 4-12% Bis-Tris gel (Life Technologies™). Samples were transferred onto a PVDF membrane (Novex by Life Technologies™) and blocked with PBST 5% non-fat dry milk. Membranes were incubated with anti-flag antibody (F1804-200UG monoclonal ANTI-FLAG® M2 antibody, SIGMA Life Sciences) and anti-β-actin antibody (Cell Signaling Technology, Inc.), and then with HRP-conjugated secondary antibodies. Blots were developed using ECL™ Prime Western Blotting Detection Reagent (GE Healthcare).

Primary Human Fibroblast Transduction

Primary human foreskin fibroblasts purchased from ATCC, CCD-1112Sk (ATCC® CRL-2429™), were seeded at a density of 1×106 cells per 10 cm³ in IMDM culture medium supplemented with 10% fetal bovine serum and penicillin/streptomycin. After 24 hours cells were infected with 1×10⁷ titration units of lentivirus in complete medium with polybrene at a concentration of 8 ng/μl and harvested 4 days later using the Qiagen AllPrep DNA/RNA Mini Kit.

Primary Human Coronary Artery Smooth Muscle Cell (hCASMC) Transduction

Primary hCASMCs were purchased from Lonza (CC-2583) and plated in 6-well plates at a density of 75,000 cells per well in culture media prepared from the Lonza Clonetics™ SmGM™-2 BulletKit™ (CC-3182). After 24 hours in culture, cells were infected with lentivirus as described for human fibroblasts.

EdU Incorporation

Primary human fibroblasts were plated in 8-well chamber slides and infected with either p16 jTALE WT or Mut lentivirus as described above. After 72 hours in culture, cells were incubated with 5-ethynyl-2′-deoxyuridine (EdU) for 1 hour. EdU incorporation was visualized by immunofluorescence staining using the Click-iT® Plus EdU Imaging Kit, Alexa Fluor® 555 picolyl azide (Molecular Probes® by Life Technologies™). Percent EdU incorporation was calculated as the number of EdU positive cells divided by the total number of cells (n=7). Differences in percent EdU incorporation were compared in a two-tailed t-test (P<0.05).

p16 Re-Expression

For p16 re-expression experiments, a lentiviral vector incorporating the human p16 cDNA clone under a CMV promoter was purchased from OriGene Technologies, Inc. (Catalog No. RC220937L1). Lentivirus was prepared by the Wistar Institute Protein Expression Core (Philadelphia, Pa.). In separate EdU Incorporation experiments, CMV-p16 lentivirus was added in combination with either p16 jTALE WT or Mut lentivirus. Titers for CMV-p16 lentivirus were reduced to approximately 1×10⁶ titration units per 1 million cells. Percent EdU incorporation was calculated as the number of EdU positive cells divided by the total number of cells (n=4). Differences in percent EdU incorporation were compared in a two-tailed t-test.

Population Doubling Time Assay

Human fibroblasts were infected as described above, except that a lower seeding density of 4×10⁵ cells per 10 cm³ plate was used to prevent contact growth inhibition during the 4 day infection period. Cells were counted prior to plating and at the end of the experiment. Population doubling time (DT) as described by ATCC was calculated as DT=T ln 2/ln(Xf/Xi) where T is incubation time (days), Xi is initial cell number and Xf is final cell number.

Statistics

Data is displayed as mean±SEM, and at least three replicates were conducted for each experiment. Data was assessed by one-way ANOVA with Tukey's correction for multiple comparisons or by two-tailed t-test, as appropriate. Significance was defined as P<0.05.

### Results

Custom TALE-DNMT Fusion Proteins Direct DNA Methylation to Target Loci

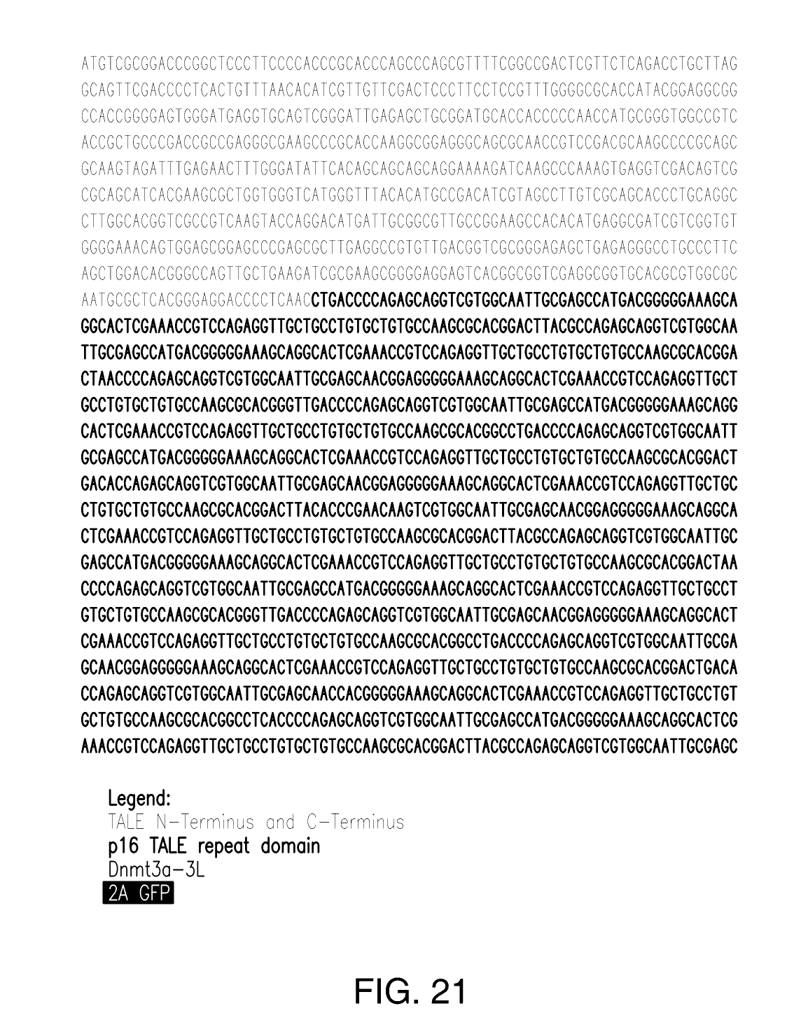

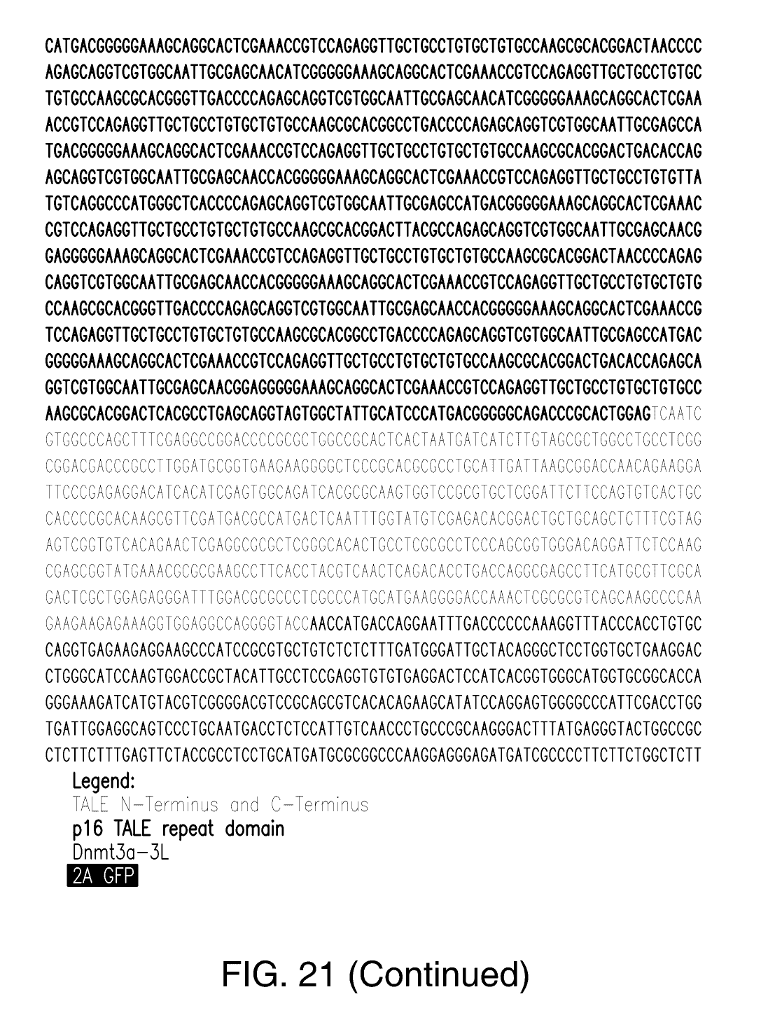

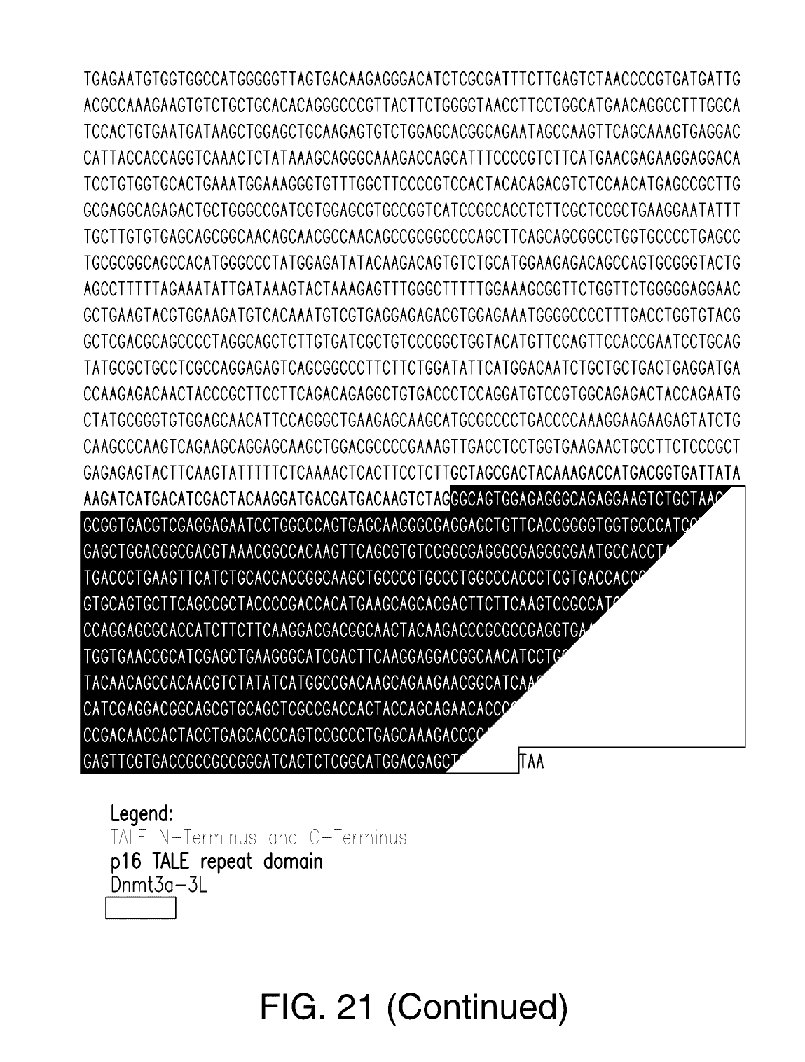

Sanjana and colleagues have described a protocol for assembly of custom TALEs using monomer templates and TALE cloning backbones (18). We modified these TALE constructs by conjugating a DNA methyltransferase catalytic subunit consisting of the C-termini of DNMT3a and DNMT3L (6) to the C-terminus of the TALE protein. The cloning backbone contains eGFP and was further modified to include a 3× Flag-tag, modifications which we employed for cell sorting and protein detection, respectively. We also constructed a catalytically inactive TALEDNMT as a negative control by introducing a point mutation in the DNMT3a subunit at the E752A position (19). TALE monomers were assembled into the backbone-cloning vector through a series of Golden Gate digestion-ligation reactions (18). Following this protocol, we constructed TALE-DNMTs targeting 24-base pair sequences within the CDKN2A promoter (20). The TALE-DNMT illustrated in FIG. 15b is engineered to bind the target sequence 5′-CCTCCTTCCTTGCCAACGCTGGCT-3′, at position −28 to −4 upstream of the p16 (CDKN2A) transcription start site. The complete coding sequence of the p16 TALE-DNMT is provided in FIG. 21.

To test our strategy, we transfected HeLa cells, a human cervical adenocarcinoma cell line, with p16 TALE-DNMT wild-type and mutant expression constructs and compared DNA methylation of the CDKN2A locus between the two transfected populations, and also to untreated HeLa cells. Cells were collected 48 hours post-transfection, and FACS-sorted for GFP to isolate transfected populations. Average transfection efficiency was 12.1% and 14.6% for the wild-type and mutant constructs, respectively. DNA methylation was then evaluated using bisulfite conversion of genomic DNA followed by PCR amplification and high throughput sequencing. Strikingly, we found that transfection of a single TALE-DNMT construct is sufficient to dramatically alter DNA methylation across the entire CpG island within the CDKN2A promoter (FIG. 15c). DNA methylation was significantly elevated in the p16 TALE-DNMT GFP positive cells compared to both mutant GFP positive and untreated cells (P<0.0001). On average, DNA methylation increased by 17% across the entire CpG island, and by as much as 66.5% at individual CpGs when comparing wild-type and mutant transfected populations (FIG. 15c). There was no difference between p16 TALE-DNMT mutant transfected and untreated populations, confirming that the E752A mutation completely eliminates the catalytic activity of the enzyme.

Despite the dramatic increase in DNA methylation at the CDKN2A promoter, p16 expression decreased by only a small amount in p16 TALE-DNMT wild-type transfected HeLa cells compared to mutant and uninfected populations (data not shown). This is likely due to the fact that p16 expression is often upregulated in cervical cancers, and may be subject to aberrant regulatory mechanisms in these transformed cells (21,22). Thus, we hypothesized that a primary human cell line might be a more suitable system to study functionality at this particular target.

Minimizing Direct Repeats in TALE Modules Permits Lentiviral Delivery

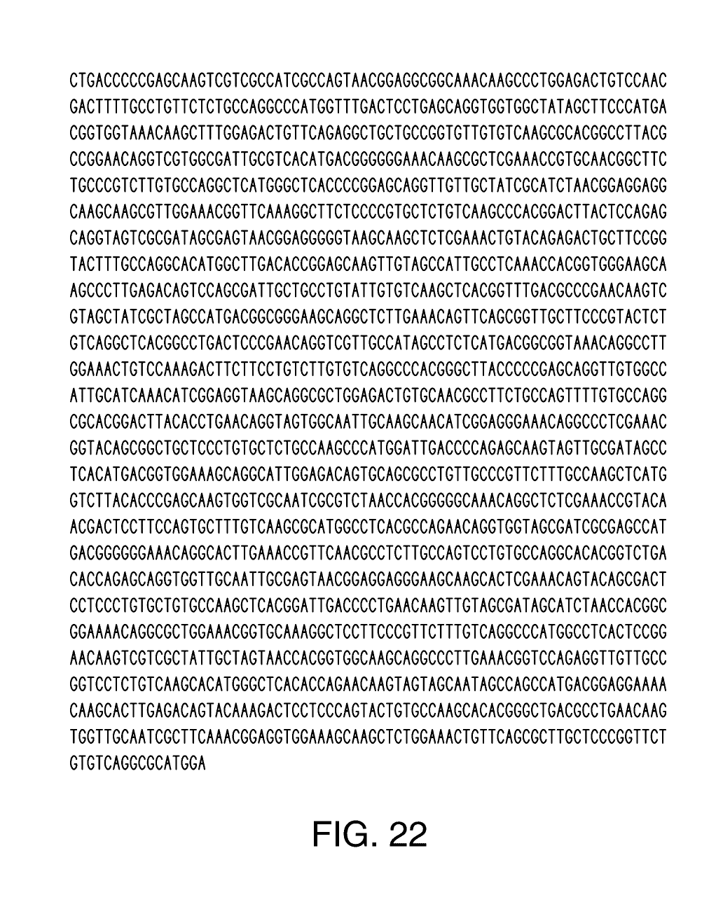

We next considered alternate strategies for gene delivery that might be more suitable to targeting primary human cells, which are often difficult to transfect even ex vivo. Lentiviral vectors provide an efficient method for stably introducing genes into host genomes of multiple cell types. However, standard TALE technology is incompatible with lentiviral delivery due to the large number of tandem repeats in the TALE moiety that lead to sequence loss by DNA recombination (13). In order to promote efficient integration of intact TALEs into host genomes using lentivirus, we utilized the degeneracy of the genetic code to minimize direct repeats across the TALE DNA binding domain to build “jumbled” TALE-DNMTs (jTALE-DNMTs—see FIG. 22), similar to the strategy pursued by Yang and colleagues (23). Furthermore, in order to accommodate the size limitation of the lentiviral genome, we made several modifications to the TALE-DNMT constructs by decreasing the number of repeats from 24 to 18, such that the revised TALE targets the sequence 5′-TCCTTGCCAACGCTGGCT-3′. We also truncated the promoter and removed the eGFP sequence.

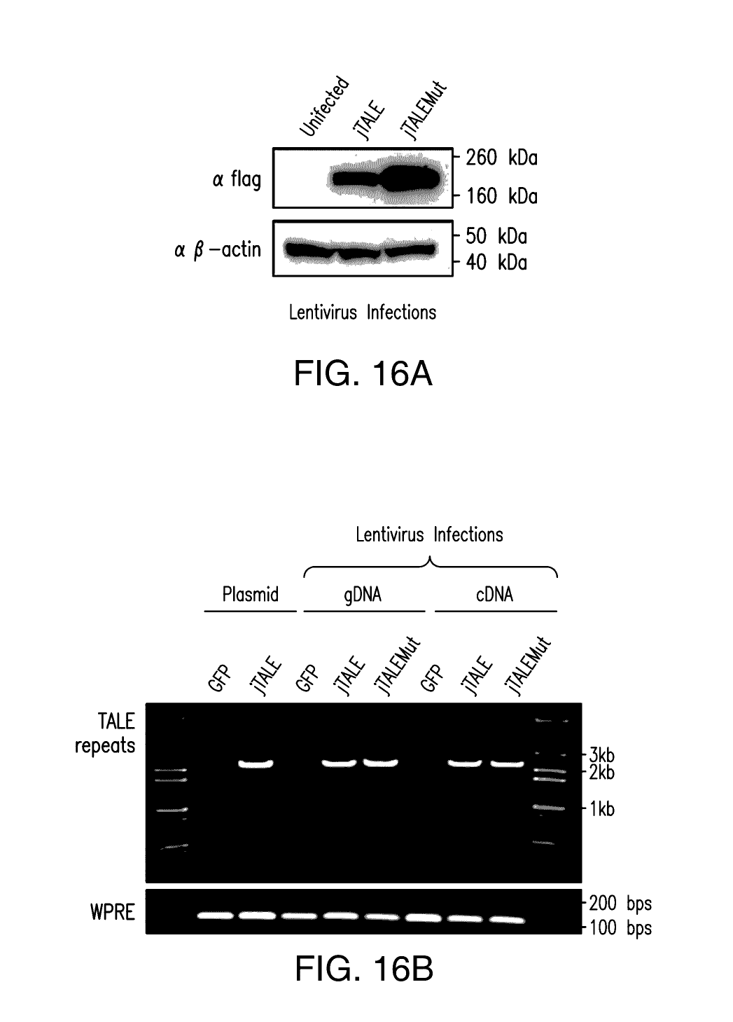

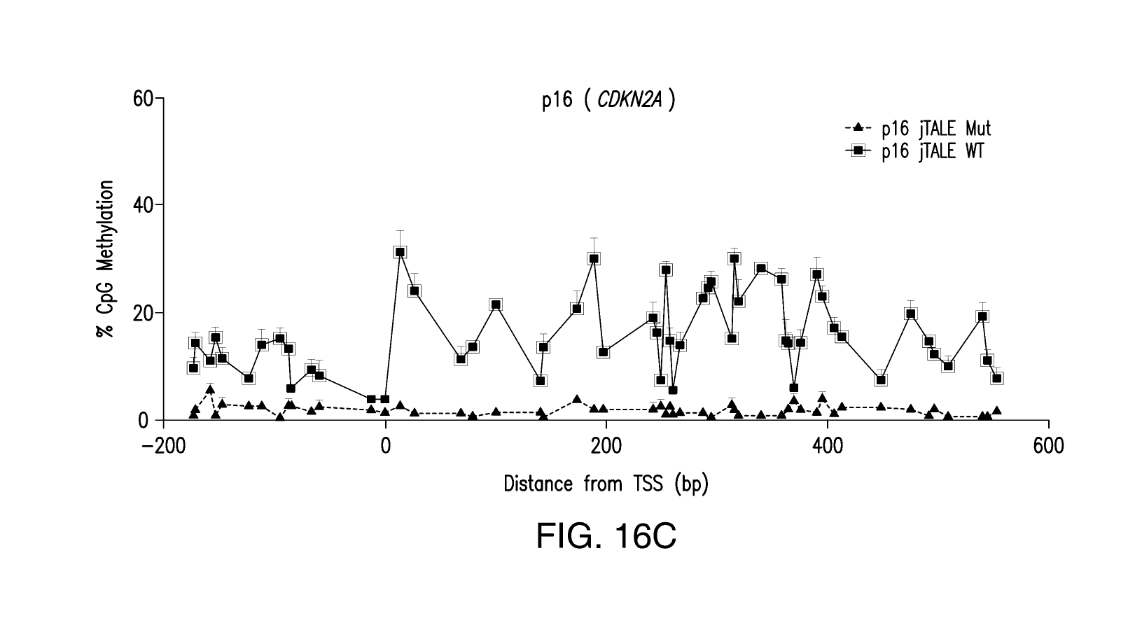

We prepared lentivirus for wild-type and mutant p16 jTALE-DNMT constructs and tested their functionality in HeLa cells. Western blots confirmed that full-length jTALE-DNMT protein was indeed produced following infection (FIG. 16a). In order to demonstrate that the entire, unrecombined jumbled TALE repeat moiety was integrated into the host genome, we PCRamplified the repeat region from genomic DNA of transduced cells. We also amplified the jTALE repeat region from cDNA to determine that the full-length repeat region was transcribed (FIG. 16b). Together, these assays confirmed integration and expression of full-length TALE constructs. We then established that p16 jTALE-DNMT lentiviruses target CpG methylation at the CDKN2A locus, with an average increase of 13.8% in the p16 jTALE-DNMT infected population compared to the mutant (FIG. 16c). The lesser extent of DNA methylation in the lentivirus infections compared to transfected cells is likely due to the fact that cells could not be sorted into infected populations. These data demonstrate convincingly that jumbled TALE repeats can be administered stably using lentiviral vectors, removing a significant obstacle for wide-spread application of the TALE technology.

Targeted DNA Methylation in Primary Human Cells Results in Decreased Gene Expression of the Target Gene

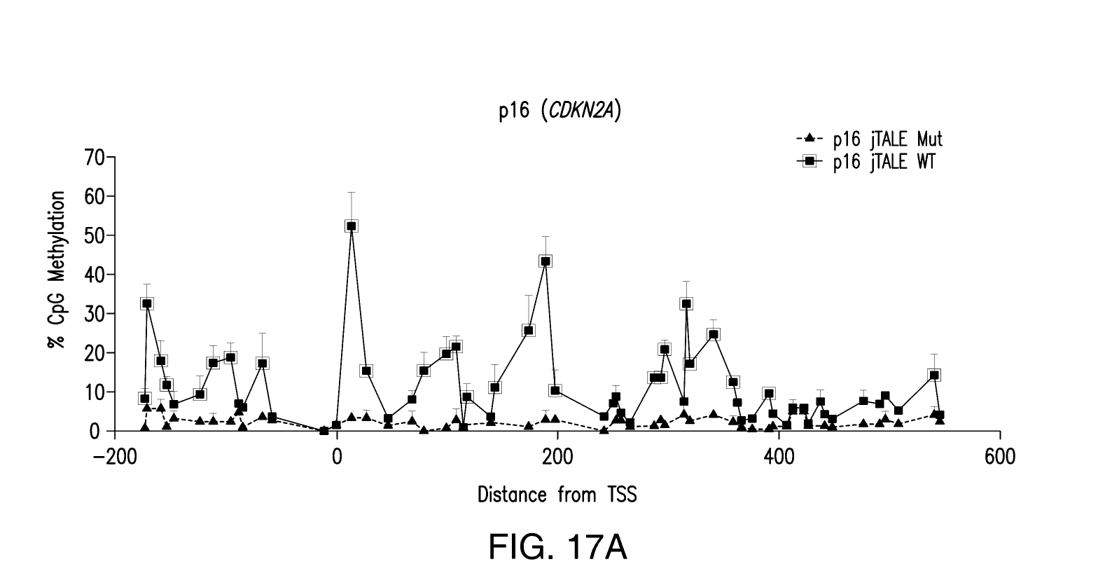

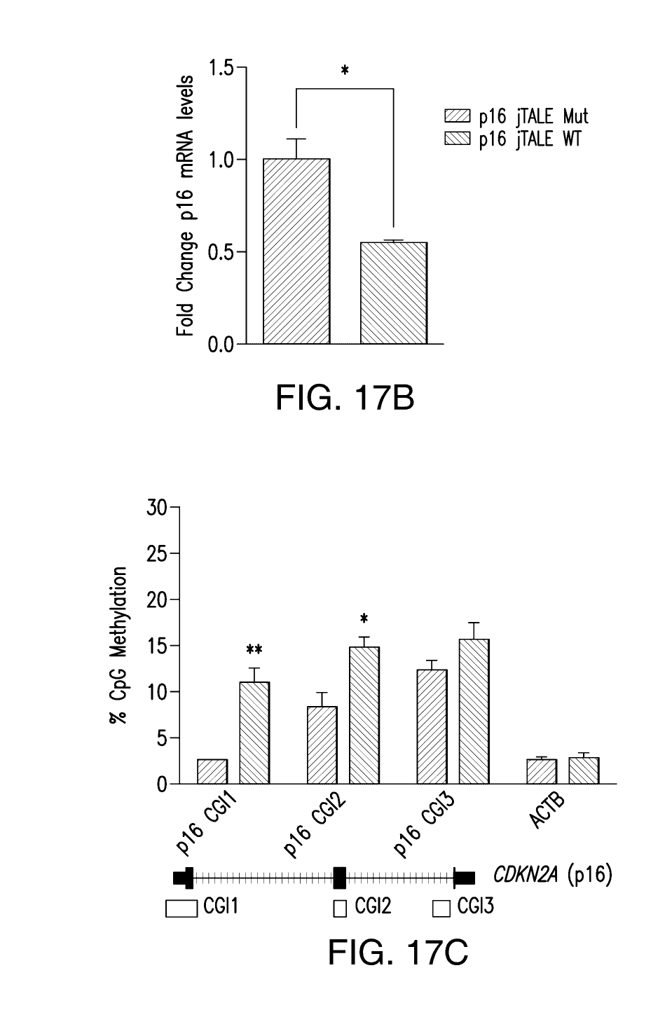

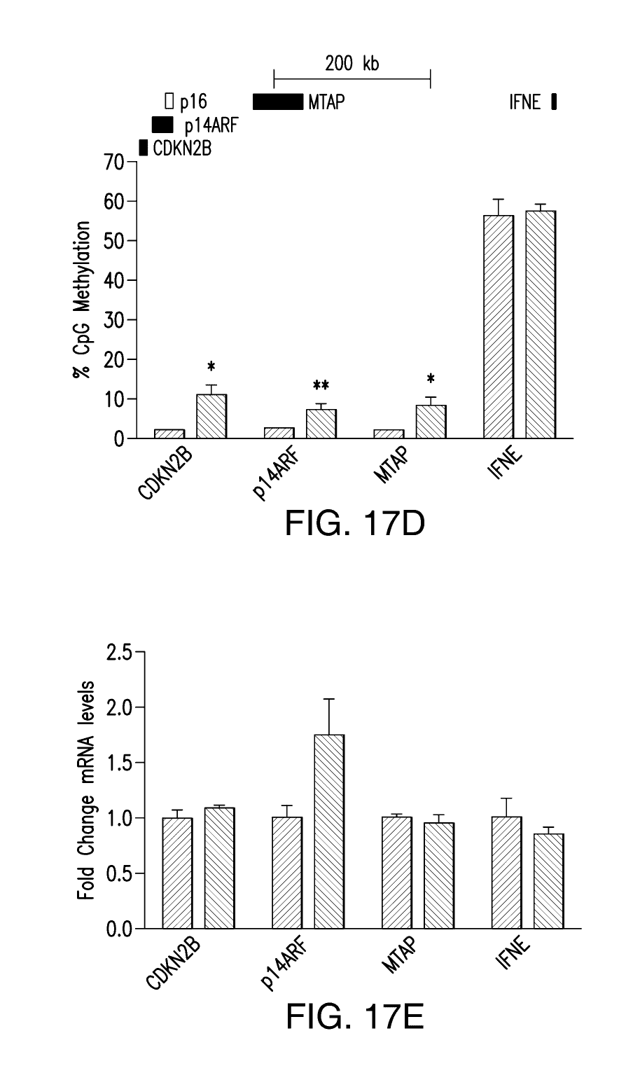

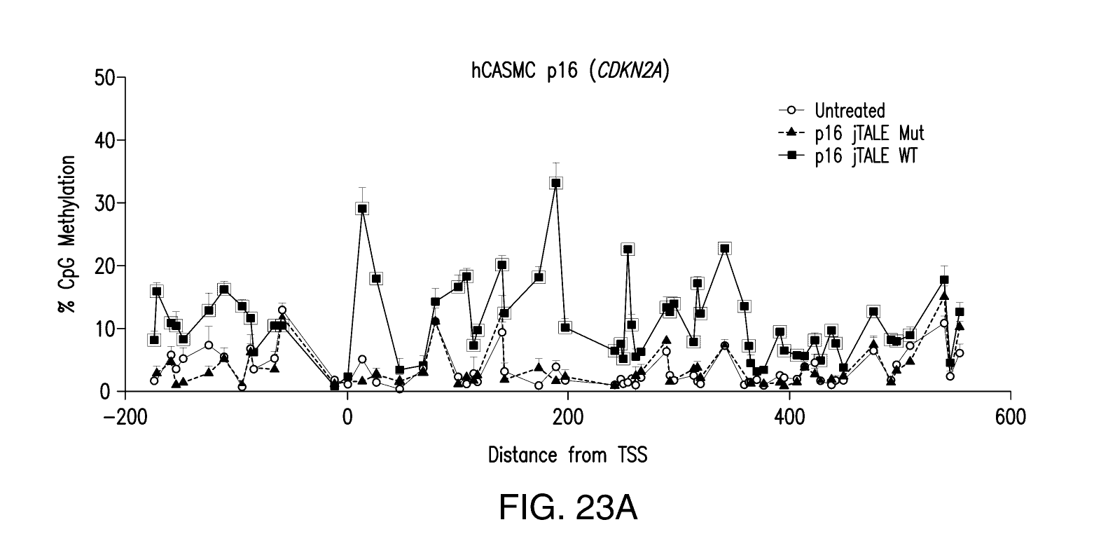

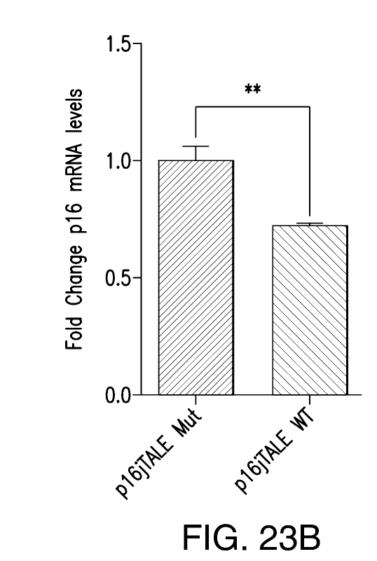

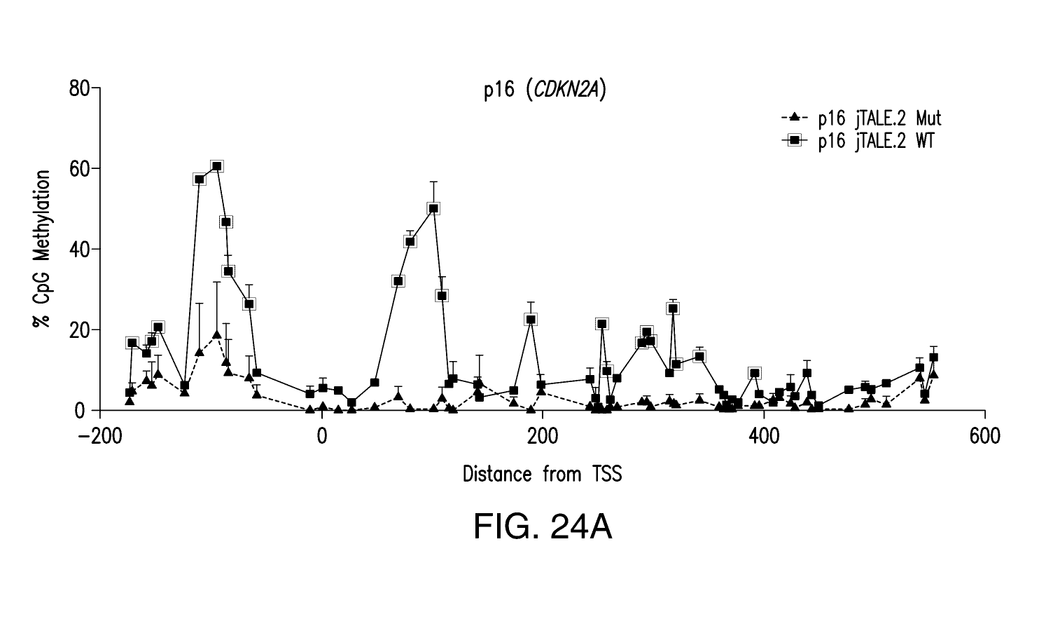

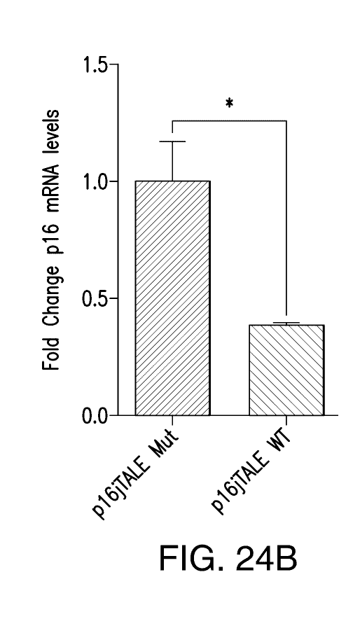

Having successfully developed jTALE-DNMTs for lentiviral delivery, we next tested if TALE-DNMTs can methylate the p16 (CDKN2A) locus in primary human fibroblasts. After infection with wild-type and mutant p16 jTALE-DNMT constructs, DNA methylation was again evaluated by sodium bisulfite conversion followed by PCR amplification and high throughput sequencing. Average DNA methylation was significantly increased by approximately 10% across the CDKN2A CpG island when comparing fibroblasts infected with wild-type and mutant p16 jTALE-DNMTs (P<0.005), with several CpGs showing increases in methylation of 30 to 50% (FIG. 17a). To evaluate the functional consequence of increased CpG methylation at this locus, we measured p16 mRNA levels and found an approximately 50% decrease in p16 mRNA levels in p16 jTALE-DNMT infected fibroblasts relative to mutant infected fibroblasts (P<0.05) (FIG. 17b). In order to validate the broad applicability of the TALE-DNMT strategy, we also tested the p16 TALE-DNMT lentiviruses in primary human coronary artery smooth muscle cells (hCASMCs), and showed that p16 jTALE-DNMT wild-type infected cells had increased DNA methylation and a corresponding decrease in p16 (CDKN2A) expression compared to mutant infected cells (see FIG. 23). As further validation of our strategy we also designed a new p16 TALE-DNMT targeted to the p16 (CDKN2A) promoter 118 to 136 base pairs upstream from the transcription start site (5′-TAACAGAGTGAACGCACT-3′). This additional TALE-DNMT, p16 TALE-DNMT.2, also increased DNA methylation and led to even stronger repression of p16 transcription when comparing wild-type and mutant infected cells (see FIG. 24). Remarkably, this stronger gene repression was associated with fewer affected CpGs, suggesting that specific CpGs are more relevant than others. In sum, the overall TALE-DNMT strategy can be extended to multiple primary human cell types and multiple target DNA binding sites.

To further characterize the effects of the p16 TALE-DNMT locally and genome-wide, we measured DNA methylation at the two other CpG islands within the p16 (CDKN2A) locus, and at β-actin, a housekeeping gene located on a different chromosome (FIG. 17c). Interestingly, we found that the effect of the TALE-DNMT on average DNA methylation decreased with distance from the transcription start site at the p16 (CDKN2A) locus, as DNA methylation increased significantly by 6% at CGI2, but not significantly at CGI3. The β-actin locus on chromosome 7 was completely unaffected.

Given the increase in DNA methylation observed at the adjacent CpG island, we considered the possibility that other nearby genes might display altered levels of DNA methylation (FIG. 17d). Indeed, we measured increased methylation in several genes adjacent to p16 (CDKN2A), including p14ARF, another transcript within the CDKN2A locus, CDKN2B, and MTAP. No changes were observed in IFNE. We next measured mRNA levels at these genes to determine the functional consequence of the increased DNA methylation and observed no changes in the mRNA levels of CDKN2B, or MTAP and IFNE (FIG. 17e). We also analyzed expression of the three other transcripts at the CDKN2A locus and did not observe a significant change in expression of p14ARF (FIG. 17e), while p12 and p16γ, were not detectable by qPCR in human fibroblasts.

DNA Methylation of the p16 (CDKN2A) Locus Results in Increased Replication in Primary Human Cells

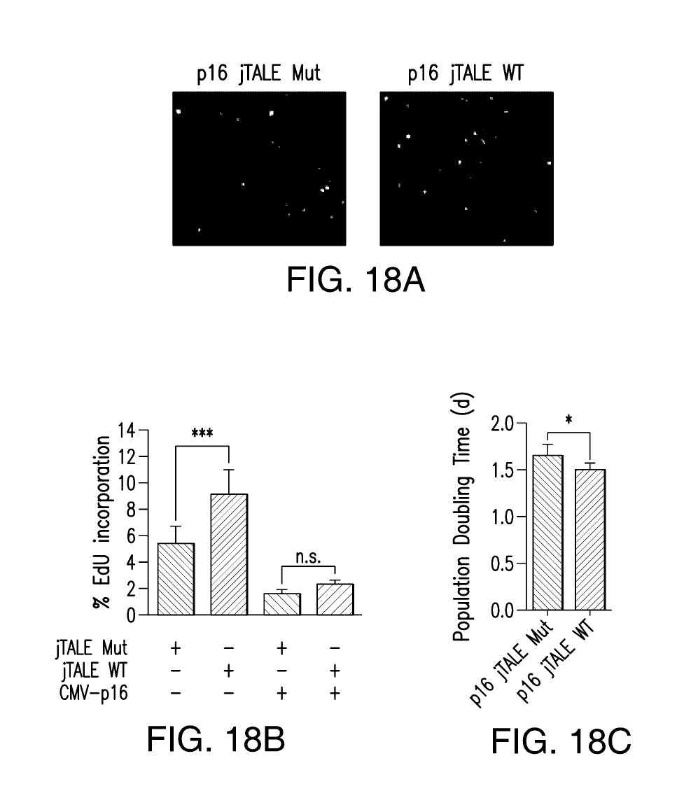

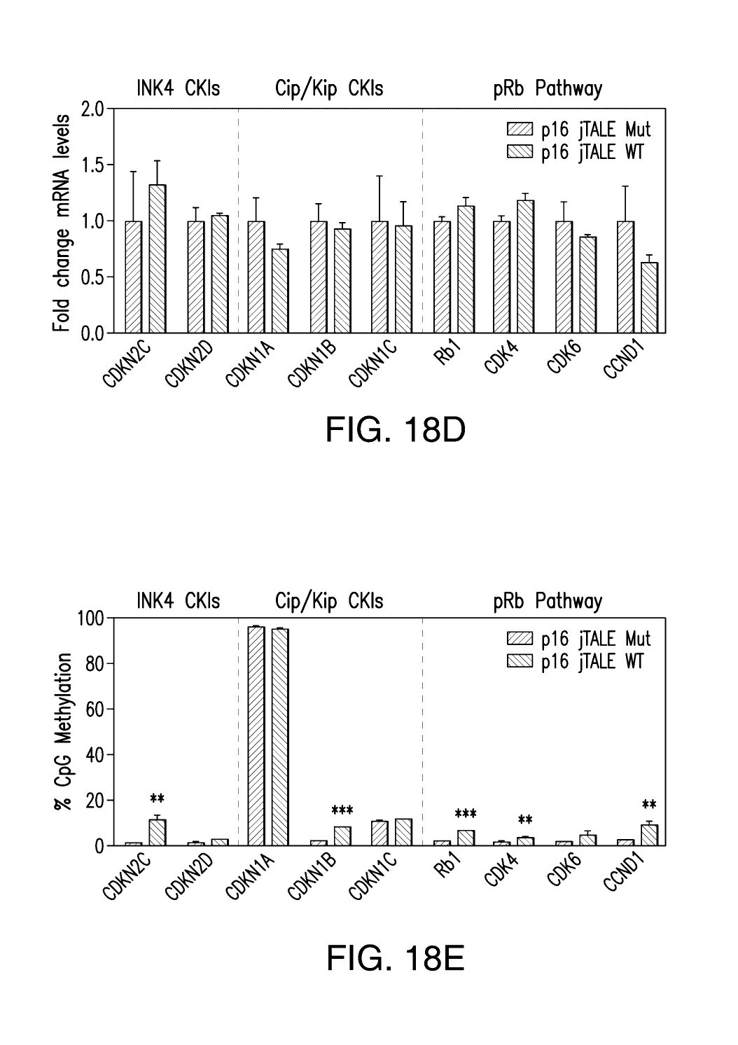

Our goal in decreasing p16 expression is to more readily permit entry into the cell cycle. Therefore, we next evaluated rates of DNA replication in primary human fibroblasts infected with wild-type and mutant p16 jTALE-DNMTs. Following infection and subsequent incubation for 72 hours, we measured incorporation of the thymidine analogue 5-ethynyl-2′-deoxyuridine (EdU) by immunofluorescence staining after a 60-minute incubation period. p16jTALE-DNMTs increased DNA replication by nearly two-fold in the wild-type compared to mutant infected populations (FIGS. 18a and b). In order to confirm that the altered proliferation observed in p16 jTALEDNMT-transduced human fibroblasts was specifically due to suppression of p16 transcription, and not some unknown off-target effect, we restored p16 levels by co-infection with a lentivirus driving p16 expression under control of the CMV promoter. As shown in FIG. 18b, reexpression of p16 completely ablated the pro-proliferative effect of p16 jTALE-DNMT activity, providing strong evidence that increased replication in human fibroblasts was indeed caused by reduced p16 levels. Since p16 mediates progression through the G1 phase of the cell cycle, we also wanted to confirm that increased DNA replication rates translated into cellular proliferation. We therefore calculated population-doubling time of infected human fibroblasts and found that this was reduced by 10% in the p16 jTALE-DNMT wild-type infected cells compared to mutant (FIG. 18c).

To further eliminate the possibility that increased proliferation of epigenetically targeted human fibroblasts might be impacted by other cell cycle regulators, we determined the expression levels of a panel of cyclin-dependent kinase inhibitors, cyclin-dependent kinases and other molecules related to the p16 inhibitory pathway. As shown in FIG. 18d, none of these cell cycle regulators were affected by p16 jTALE-DNMT infection comparing wild-type and mutant infected populations, attesting to the specificity of our strategy. Although we demonstrated that p16 TALE-DNMT infection does not affect expression of other cell cycle regulators, we also assessed DNA methylation at these loci. We found that some genes showed increased methylation while others did not (FIG. 18e). Since changes across cell cycle regulators are inconsistent, we cannot say for certain whether or not these changes are a direct consequence of p16 TALE-DNMT activity, or an indirect consequence of decreased p16 expression and accelerated proliferation. In summary, targeting DNA methylation to the p16 (CDKN2A) promoter led to changes in expression exclusively of the p16 transcript, resulting in increased cell proliferation, without altering the activity of other cell cycle regulator genes.

### Discussion

We have demonstrated that customized TALEs can be employed to direct DNA methylation to specific gene loci, and thereby decrease gene expression. We selected p16 as our primary target due to its role in mediating cellular senescence with the idea that epigenetic suppression of p16 might facilitate cell cycle entry in terminally differentiated cells in the context of regenerative medicine. In developing this novel technology, we employed a primary human fibroblast cell line that is not transformed, in contrast to the majority of human cells lines, which are often subject to dysfunctional cell cycle regulation. Importantly, even in this readily dividing cell population we have shown that increasing DNA methylation at a single cell cycle inhibitor gene locus is sufficient to increase cellular proliferation. Further, the observed changes in replication rate were entirely dependent on suppression of the intended gene product, p16. We have also proven that these targeted epigenetic modifiers can be delivered using lentiviral vectors, which will dramatically expand the breadth of their application in different biological systems. We validated our findings of TALE-DNMT mediated targeted DNA methylation and p16 suppression in primary human coronary artery smooth muscle cells, and with an independent TALE-DNMT construct targeting the p16 (CDKN2A) locus, attesting to the broad applicability of this technology. We have shown that the TALE-DNMT strategy is a powerful and robust tool that can be utilized to change cell fate.

We also found that nearby loci are more likely to be susceptible to off-target effects by TALE-DNMTs than distal loci, which until now has been an underreported consequence of TALE-mediated epigenetic modifiers. These “near-target” effects appear to be distance dependent, and in our case functionally irrelevant as altered gene expression was detected only at the target gene, CDKN2A. We also observed small increases in DNA methylation at some but not all cell cycle inhibitors and other cell cycle regulators implicated in the p16/pRb pathway. Critically, these changes did not impact gene activity. Nevertheless, future efforts will be directed towards further improving specificity by strategies such as attenuating DNMT subunit catalytic activity, or splitting the DNMT domain, similar to the TALEN system, such that two separate TALE fusion proteins are required for methyltransferase activity. Also, the TALE-DNMT strategy may be further optimized through large-scale screens of TALEs to determine if certain target binding sites are more effective than others.

While there are several techniques being developed for directing epigenetic modifiers to specific genomic loci, they differ in their DNA targeting strategies. Current approaches include zinc fingers, TALEs, and clustered regularly interspaced short palindromic repeat (CRISPR)/CRISPR associated protein 9 (Cas9). Zinc fingers represent one of the earliest examples of engineered DNA binding proteins, and have been coupled to a wide range of effector domains, including DNA methyltransferases, as discussed earlier. Zinc finger DNA binding modules interact with a series of three base pairs, somewhat limiting potential target sequences. Therefore, TALEs have largely replaced zinc fingers in these efforts since they are extremely modular, inexpensive, and quick to make. The CRISPR/Cas9 system has emerged as an exciting new tool for genome editing, as the Cas9 nuclease can be directed to target DNA sequences by 20-base pair small-guide RNAs (sgRNAs), eliminating the need for engineering sequence-specific DNA binding proteins. While most work to date has focused on genomic engineering, the field is rapidly evolving into the gene expression space, using a catalytically inactive or “dead” variant of the Cas9 nuclease, dCas9. In fact, the concept of using Cas9 towards targeted epigenetic modifications has already been proposed, although not yet demonstrated (24). Despite differences in underlying mechanisms, any of these systems are subject to potential off-target effects, and improving specificity is an ongoing challenge in the field. The preferred method can be determined in a context-dependent manner.

Here, we demonstrate the utility of TALE-directed DNA methylation as a strategy for altering the epigenetic state in a targeted, locus-specific fashion. We have amended this strategy to accommodate lentiviral delivery to primary human cells, and have shown that this system can be used to alter cellular behavior. We also shed light on the specificity of TALE mediated epigenetic targeting, which is an ongoing area of future investigation. This study may have widespread implications for investigating gene regulation, and in developing novel therapeutic strategies for correcting aberrant gene expression in disease.

### REFERENCES

1. Heerboth S, Lapinska K, Snyder N, Leary M, Rollinson S, Sarkar S. Use of epigenetic drugs in disease: An overview. Genetics & Epigenetics. 2014; 6:9-19.
2. Cacabelos R. Epigenomic networking in drug development: From pathogenic mechanisms to pharmacogenomics. Drug Dev Res. 2014; 75(6):348-365.
3. Jones P A. Functions of DNA methylation: Islands, start sites, gene bodies and beyond. Nat Rev Genet. 2012; 13(7):484-492.
4. Bergman Y, Cedar H. DNA methylation dynamics in health and disease. Nature Structural & Molecular Biology. 2013; 20(3):274-281.
5. Sheaffer K L, Kim R, Aoki R, et al. DNA methylation is required for the control of stem cell differentiation in the small intestine. Genes Dev. 2014; 28(6):652-664.
6. Siddique A N, Nunna S, Rajavelu A, et al. Targeted methylation and gene silencing of VEGFA in human cells by using a designed Dnmt3α-Dnmt3L single-chain fusion protein with increased DNA methylation activity. J Mol Biol. 2013; 425(3):479-491.
7. Bogdanove A J, Voytas D F. TAL effectors: Customizable proteins for DNA targeting. Science. 2011; 333(6051):1843-1846.
8. Cermak T, Doyle E L, Christian M, et al. Efficient design and assembly of custom TALEN and other TAL effector-based constructs for DNA targeting (vol 39, pg e82, 2011). Nucleic Acids Res. 2011; 39(17):7879-7879.
9. Morbitzer R, Romer P, Boch J, Lahaye T. Regulation of selected genome loci using de novo engineered transcription activator-like effector (TALE)-type transcription factors. PNAS. 2010; Volume: 107(Issue: 50):21617-21622.
10. Cong L, Zhou R, Kuo Y, Cunniff M, Zhang F. Comprehensive interrogation of natural TALE DNA-binding modules and transcriptional repressor domains. Nature Communications. 2012; 3:968.
11. Maeder M L, Angstman J F, Richardson M E, et al. Targeted DNA demethylation and activation of endogenous genes using programmable TALE-TET1 fusion proteins. Nat Biotechnol. 2013; 31(12):1137.
12. Mendenhall E M, Williamson K E, Reyon D, et al. Locus-specific editing of histone modifications at endogenous enhancers. Nat Biotechnol. 2013; 31(12): 1133-+.
13. Holkers M, Maggio I, Liu J, et al. Differential integrity of TALE nuclease genes following adenoviral and lentiviral vector gene transfer into human cells. Nucleic Acids Res. 2013; 41(5):e63.
14. Martin N, Beach D, Gil J. Ageing as developmental decay: Insights from p16INK4a. Trends in Molecular Medicine. 2014; 20(12):667-674.
15. Jeck W R, Siebold A P, Sharpless N E. Review: A meta-analysis of GWAS and ageassociated diseases. Aging Cell. 2012; 11(5):727-731.
16. Herman J G, Merlo A, Mao L, et al. Inactivation of the CDKN2/p16/MTS1 gene is frequently associated with aberrant DNA methylation in all common human cancers. Cancer Res. 1995; 55((20)):4525-30. Krishnamurthy J, Ramsey M R, Ligon K L, et al. p16(INK4a) induces an age-dependent decline in islet regenerative potential. Nature. 2006; 443(7110):453-457.
18. Sanjana N E, Cong L, Zhou Y, Cunniff M M, Feng G, Zhang F. A transcription activator-like effector toolbox for genome engineering. Nature Protocols. 2012; 7(1):171-192.
19. Rivenbark A G, Stolzenburg S, Beltran A S, et al. Epigenetic reprogramming of cancer cells via targeted DNA methylation. Epigenetics. 2012; 7(4):350-360.
20. Kent W J, Sugnet C W, Furey T S, et al. The human genome browser at UCSC. Genome Res. 2002; 12(6):996-1006.
21. Ivanova T A, Golovina D A, Zavalishina L E, et al. Up-regulation of expression and lack of 5′ CpG island hypermethylation of p16 INK4a in HPV-positive cervical carcinomas. BMC Cancer. 2007; 7:47.
22. McLaughlin-Drubin M, Park D, Munger K. Tumor suppressor p16INK4A is necessary for survival of cervical carcinoma cell lines. Proc Natl Acad Sci USA. 2013; 110(40):16175-16180.
23. Yang L, Guell M, Byrne S, et al. Optimization of scarless human stem cell genome editing. Nucleic Acids Res. 2013; 41(19):9049-9061.
24. Sander J D, Joung J K. CRISPR-cas systems for editing, regulating and targeting genomes. Nat Biotechnol. 2014; 32(4):347-355.
25. Doyle E L, Booher N J, Standage D S, et al. TAL effector-nucleotide targeter (TALE-NT) 2.0: Tools for TAL effector design and target prediction. Nucleic Acids Res. 2012; 40(W1):W117-W122.
26. Chen P, Cokus S J, Pellegrini M. B S seeker: Precise mapping for bisulfite sequencing. BMC Bioinformatics. 2010; 11:203.

Various publications, patents, patent application, and GenBank Accession Nos. are cited herein, the contents of which are hereby incorporated by reference in their entireties.

1. A method of treating or preventing diabetes in a subject, comprising administering to the subject a therapeutically effective amount of a pharmaceutical composition comprising an epigenetic modifier capable of reprogramming an α-cell cell into a functional β-cell.

2. The method of claim 1, wherein the subject is a human.

3. The method of claim 1, wherein the epigenetic modifier is a small molecule.

4. The method of claim 1, wherein the epigenetic modifier is a nucleic acid molecule.

5. The method of claim 4, wherein the epigenetic modifier nucleic acid molecule is expressed by a therapeutic vector.

6. The method of claim 5, wherein a pancreatic cell from the subject is transduced with the therapeutic vector in vitro and reintroduced into the subject.

7. The method of claim 1, wherein the epigenetic modifier is a polypeptide complex.

8. The method of claim 1, wherein the epigenetic modifier is administered in conjunction with one or more additional agents for the treatment or prevention of diabetes.

9. The method of claim 4, wherein the nucleic acid molecule comprises:
a. one or more coding sequences operably linked to a promoter sequence,
b. wherein the one or more coding sequences encode at least a first polypeptide domain comprising a TAL effector DNA binding domain and at least a second polypeptide domain having epigenetic modifying activity.

10. The method of claim 10, wherein the first polypeptide domain is specifically directed towards binding to one or more nucleic acid sequences in a target gene that are involved in the control of gene expression.

11. The method of claim 10, wherein the second polypeptide domain is a catalytic domain of a histone-modifying protein.

12. The method of claim 10, wherein the second polypeptide domain is a catalytic domain selected from the group consisting of: a histone methyltransferase; a histone demethylase; a histone acetyltransferase; a histone deacetylase; a nucleic acid methyltransferase; and a nucleic acid demethylase.

13. The method of claim 7, wherein the polypeptide complex comprises:
a. at least a first domain comprising a TAL effector DNA binding domain; and
b. at least a second domain having epigenetic modifying activity.

14. The method of claim 14, wherein the first domain is specifically directed towards binding to one or more nucleic acid sequences in a target gene that are involved in the control of gene expression.

15. The method of claim 14, wherein the second domain is a catalytic domain selected from the group consisting of: a histone methyltransferase; a histone demethylase; a histone acetyltransferase; a histone deacetylase; a nucleic acid methyltransferase; and a nucleic acid demethylase.

16. The method of claim 14, wherein the second domain is a catalytic domain of a histone-modifying protein.

17. The method of claim 14, wherein the second domain is capable of methylating a lysine residue located at position 27 in the tail region of histone H3 (H3K27me3).

18. The method of claim 10 or 14, wherein the target gene is selected from the group consisting of: Pdx1, Pax4, Arx, Dpp4, Ptprd, and MafA.

19. A method for directing DNA methylation to a specific genetic locus in a cell comprising administering to said cell, a transcription activator-like effector (TALE)-methylation domain conjugate.

20. The method of claim 19, wherein the cell is a human cell.

21. The method of claim 19, wherein the TALE-methylation domain conjugate is a TALE-DMNT conjugate.

22. The method of claim 1, wherein the TALE-methylation domain conjugate is expressed by a therapeutic vector.

23. The method of claim 22, wherein the TALE-methylation domain conjugate expressed by a therapeutic vector is a TALE-DMNT conjugate.

24. The method of claim 23, wherein a cell is transduced with the therapeutic vector in vitro and reintroduced into a subject.

25. The method of claim 19, wherein the TALE-methylation domain conjugate is administered in conjunction with one or more additional agents.

26. The method of claim 22, wherein the TALE-methylation domain conjugate is encoded by a nucleic acid molecule comprising:
a. one or more coding sequences operably linked to a promoter sequence,
b. wherein the one or more coding sequences encode at least a first polypeptide domain comprising a TAL effector DNA binding domain and at least a second polypeptide domain having methylation activity.

27. The method of claim 26, wherein the first polypeptide domain is specifically directed towards binding to one or more nucleic acid sequences in a target gene that are involved in the control of gene expression.


SEQUENCE LISTING (86 sequences)

SEQ ID NO: 1 - DNA, 24 bp, Artificial Sequence
  Description of Artificial Sequence Synthetic oligonucleotide
cctccttccttgccaacgctggct

SEQ ID NO: 2 - DNA, 20 bp, Artificial Sequence
  Description of Artificial Sequence Synthetic primer
caagccccaagaagaagaga

SEQ ID NO: 3 - DNA, 26 bp, Artificial Sequence
  Description of Artificial Sequence Synthetic primer
cccatggccaccacattggcaaagag

SEQ ID NO: 4 - DNA, 18 bp, Artificial Sequence
  Description of Artificial Sequence Synthetic oligonucleotide
tccttgccaacgctggct

SEQ ID NO: 5 - DNA, 23 bp, Artificial Sequence
  Description of Artificial Sequence Synthetic primer
ccagttgctgaagatcgcgaagc

SEQ ID NO: 6 - DNA, 22 bp, Artificial Sequence
  Description of Artificial Sequence Synthetic primer
tgccactcgatgtgatgtcctc

SEQ ID NO: 7 - DNA, 20 bp, Artificial Sequence
  Description of Artificial Sequence Synthetic primer
agcgtcgacaatcaacctct

SEQ ID NO: 8 - DNA, 20 bp, Artificial Sequence
  Description of Artificial Sequence Synthetic primer
ggcattaaagcagcgtatcc

SEQ ID NO: 9 - DNA, 18 bp, Artificial Sequence
  Description of Artificial Sequence Synthetic oligonucleotide
taacagagtgaacgcact

SEQ ID NO: 10 - DNA, 20 bp, Artificial Sequence
  Description of Artificial Sequence Synthetic oligonucleotide
tacattatcggtccaatccg

SEQ ID NO: 11 - DNA, 23 bp, Artificial Sequence
  Description of Artificial Sequence Synthetic primer
atttggtagttaggaaggttgta

SEQ ID NO: 12 - DNA, 28 bp, Artificial Sequence
  Description of Artificial Sequence Synthetic primer
ccctaaaatccccccaaattaaatctcc

SEQ ID NO: 13 - DNA, 18 bp, Artificial Sequence
  Description of Artificial Sequence Synthetic primer
ggtggggaggagtttagt

SEQ ID NO: 14 - DNA, 18 bp, Artificial Sequence
  Description of Artificial Sequence Synthetic primer
aaccaacccctcctcttt

SEQ ID NO: 15 - DNA, 21 bp, Artificial Sequence
  Description of Artificial Sequence Synthetic primer
ggggttggttggttattagag

SEQ ID NO: 16 - DNA, 26 bp, Artificial Sequence
  Description of Artificial Sequence Synthetic primer
cctaattccaattcccctacaaactt

SEQ ID NO: 17 - DNA, 18 bp, Artificial Sequence
  Description of Artificial Sequence Synthetic primer
agagggggagagtaggta

SEQ ID NO: 18 - DNA, 22 bp, Artificial Sequence
  Description of Artificial Sequence Synthetic primer
ccaaaaaacctcccctttttcc

SEQ ID NO: 19 - DNA, 24 bp, Artificial Sequence
  Description of Artificial Sequence Synthetic primer
gtatggttattgtttttggtgttt

SEQ ID NO: 20 - DNA, 26 bp, Artificial Sequence
  Description of Artificial Sequence Synthetic primer
accattctattctctctaacaaatca

SEQ ID NO: 21 - DNA, 23 bp, Artificial Sequence
  Description of Artificial Sequence Synthetic primer
aggttaaagatgtgtggtatatt

SEQ ID NO: 22 - DNA, 26 bp, Artificial Sequence
  Description of Artificial Sequence Synthetic primer
acaaccactaaaactcattatataac

SEQ ID NO: 23 - DNA, 26 bp, Artificial Sequence
  Description of Artificial Sequence Synthetic primer
aatgagttttagtggttgtttataat

SEQ ID NO: 24 - DNA, 26 bp, Artificial Sequence
  Description of Artificial Sequence Synthetic primer
aacacataaataaataaacatccatt

SEQ ID NO: 25 - DNA, 28 bp, Artificial Sequence
  Description of Artificial Sequence Synthetic primer
tgggagtttagtgtttaagagatgttta

SEQ ID NO: 26 - DNA, 19 bp, Artificial Sequence
  Description of Artificial Sequence Synthetic primer
ccctctcccctccttttac

SEQ ID NO: 27 - DNA, 19 bp, Artificial Sequence
  Description of Artificial Sequence Synthetic primer
aggaggggtagtgaggatt

SEQ ID NO: 28 - DNA, 19 bp, Artificial Sequence
  Description of Artificial Sequence Synthetic primer
acactcttcccttctttcc

SEQ ID NO: 29 - DNA, 22 bp, Artificial Sequence
  Description of Artificial Sequence Synthetic primer
gggtaatgaagttgagtttagg

SEQ ID NO: 30 - DNA, 19 bp, Artificial Sequence
  Description of Artificial Sequence Synthetic primer
caccttctccactaatccc

SEQ ID NO: 31 - DNA, 25 bp, Artificial Sequence
  Description of Artificial Sequence Synthetic primer
tgggaatagtaaaagtagggtaagg

SEQ ID NO: 32 - DNA, 18 bp, Artificial Sequence
  Description of Artificial Sequence Synthetic primer
acacccacccactcaaac

SEQ ID NO: 33 - DNA, 19 bp, Artificial Sequence
  Description of Artificial Sequence Synthetic primer
ggaaaggtttgtaaaaggg

SEQ ID NO: 34 - DNA, 23 bp, Artificial Sequence
  Description of Artificial Sequence Synthetic primer
aataccaaaaaccatatctacac

SEQ ID NO: 35 - DNA, 26 bp, Artificial Sequence
  Description of Artificial Sequence Synthetic primer
gaaaagggaatttgaaaatttaatgt

SEQ ID NO: 36 - DNA, 25 bp, Artificial Sequence
  Description of Artificial Sequence Synthetic primer
aataaaaaccaacaacaccaacaca

SEQ ID NO: 37 - DNA, 24 bp, Artificial Sequence
  Description of Artificial Sequence Synthetic primer
ggaggatttgtttttgtagttttg

SEQ ID NO: 38 - DNA, 25 bp, Artificial Sequence
  Description of Artificial Sequence Synthetic primer
cacaaacacacatacattcctaatt

SEQ ID NO: 39 - DNA, 25 bp, Artificial Sequence
  Description of Artificial Sequence Synthetic primer
tttatagggtttgtataattagtgg

SEQ ID NO: 40 - DNA, 19 bp, Artificial Sequence
  Description of Artificial Sequence Synthetic primer
ctacctcccttccctcaaa

SEQ ID NO: 41 - DNA, 23 bp, Artificial Sequence
  Description of Artificial Sequence Synthetic primer
gattagttggaaggagtgagaga

SEQ ID NO: 42 - DNA, 26 bp, Artificial Sequence
  Description of Artificial Sequence Synthetic primer
actcaactactccctatccactaaac

SEQ ID NO: 43 - DNA, 18 bp, Artificial Sequence
  Description of Artificial Sequence Synthetic primer
gggtagagttgggggtag

SEQ ID NO: 44 - DNA, 22 bp, Artificial Sequence
  Description of Artificial Sequence Synthetic primer
acaaacctactctaactaacct

SEQ ID NO: 45 - DNA, 22 bp, Artificial Sequence
  Description of Artificial Sequence Synthetic primer
ggtttggtttaaggttgagaag

SEQ ID NO: 46 - DNA, 18 bp, Artificial Sequence
  Description of Artificial Sequence Synthetic primer
aaccctcacacacctact

SEQ ID NO: 47 - DNA, 24 bp, Artificial Sequence
  Description of Artificial Sequence Synthetic primer
gtgattttaaaaggttagtaagtg

SEQ ID NO: 48 - DNA, 18 bp, Artificial Sequence
  Description of Artificial Sequence Synthetic primer
aaacctcatccctatccc

SEQ ID NO: 49 - DNA, 20 bp, Artificial Sequence
  Description of Artificial Sequence Synthetic primer
ggtgagggggtttttttagt

SEQ ID NO: 50 - DNA, 19 bp, Artificial Sequence
  Description of Artificial Sequence Synthetic primer
acccttccataaccaactc

SEQ ID NO: 51 - DNA, 18 bp, Artificial Sequence
  Description of Artificial Sequence Synthetic primer
ggtttgggagagggtagg

SEQ ID NO: 52 - DNA, 18 bp, Artificial Sequence
  Description of Artificial Sequence Synthetic primer
atccctcctcttccctcc

SEQ ID NO: 53 - DNA, 25 bp, Artificial Sequence
  Description of Artificial Sequence Synthetic primer
attataggggagttttgttgaagtt

SEQ ID NO: 54 - DNA, 26 bp, Artificial Sequence
  Description of Artificial Sequence Synthetic primer
caacacaaaaactaatattccataac

SEQ ID NO: 55 - DNA, 20 bp, Artificial Sequence
  Description of Artificial Sequence Synthetic primer
gcagactttgctttccttgg

SEQ ID NO: 56 - DNA, 20 bp, Artificial Sequence
  Description of Artificial Sequence Synthetic primer
aacacttcgtggggtccttt

SEQ ID NO: 57 - DNA, 19 bp, Artificial Sequence
  Description of Artificial Sequence Synthetic primer
cccaacgcaccgaatagtt

SEQ ID NO: 58 - DNA, 18 bp, Artificial Sequence
  Description of Artificial Sequence Synthetic primer
agcagcagctccgccact

SEQ ID NO: 59 - DNA, 18 bp, Artificial Sequence
  Description of Artificial Sequence Synthetic primer
agaacatggtgcgcaggt

SEQ ID NO: 60 - DNA, 20 bp, Artificial Sequence
  Description of Artificial Sequence Synthetic primer
gggatgtgaaccacgaaaac

SEQ ID NO: 61 - DNA, 20 bp, Artificial Sequence
  Description of Artificial Sequence Synthetic primer
gctgtttcatcagcagccta

SEQ ID NO: 62 - DNA, 20 bp, Artificial Sequence
  Description of Artificial Sequence Synthetic primer
ccacaatggagctagaagca

SEQ ID NO: 63 - DNA, 20 bp, Artificial Sequence
  Description of Artificial Sequence Synthetic primer
ctacctcagggcatggttgt

SEQ ID NO: 64 - DNA, 20 bp, Artificial Sequence
  Description of Artificial Sequence Synthetic primer
tgctggctaaatgcttgttg

SEQ ID NO: 65 - DNA, 20 bp, Artificial Sequence
  Description of Artificial Sequence Synthetic primer
acccttgaacgacatgaagc

SEQ ID NO: 66 - DNA, 23 bp, Artificial Sequence
  Description of Artificial Sequence Synthetic primer
aagaatcatgtctggagaagtcc

SEQ ID NO: 67 - DNA, 20 bp, Artificial Sequence
  Description of Artificial Sequence Synthetic primer
gtgatttggccaggctctat

SEQ ID NO: 68 - DNA, 19 bp, Artificial Sequence
  Description of Artificial Sequence Synthetic primer
agccctccccacgtttatt

SEQ ID NO: 69 - DNA, 20 bp, Artificial Sequence
  Description of Artificial Sequence Synthetic primer
ccaagggcagagcatttaag

SEQ ID NO: 70 - DNA, 20 bp, Artificial Sequence
  Description of Artificial Sequence Synthetic primer
aagcaacgtgcacacttcag

SEQ ID NO: 71 - DNA, 20 bp, Artificial Sequence
  Description of Artificial Sequence Synthetic primer
gattagcagcggaacaagga

SEQ ID NO: 72 - DNA, 20 bp, Artificial Sequence
  Description of Artificial Sequence Synthetic primer
caactactcccagccccata

SEQ ID NO: 73 - DNA, 20 bp, Artificial Sequence
  Description of Artificial Sequence Synthetic primer
gaggtgcttgggagttttga

SEQ ID NO: 74 - DNA, 20 bp, Artificial Sequence
  Description of Artificial Sequence Synthetic primer
tgtttacacagcccgaagtg

SEQ ID NO: 75 - DNA, 20 bp, Artificial Sequence
  Description of Artificial Sequence Synthetic primer
cagcgttcggttttgttttt

SEQ ID NO: 76 - DNA, 20 bp, Artificial Sequence
  Description of Artificial Sequence Synthetic primer
gggaccagtgtaccttctcg

SEQ ID NO: 77 - DNA, 23 bp, Artificial Sequence
  Description of Artificial Sequence Synthetic primer
agggcttactatttctgggtctt

SEQ ID NO: 78 - DNA, 23 bp, Artificial Sequence
  Description of Artificial Sequence Synthetic primer
caatacacgatctctgaagttcc

SEQ ID NO: 79 - DNA, 20 bp, Artificial Sequence
  Description of Artificial Sequence Synthetic primer
cgagctctgcagcactctta

SEQ ID NO: 80 - DNA, 20 bp, Artificial Sequence
  Description of Artificial Sequence Synthetic primer
agaagggaaatggcagcttt

SEQ ID NO: 81 - DNA, 20 bp, Artificial Sequence
  Description of Artificial Sequence Synthetic primer
tgtctatctcccggcacttc

SEQ ID NO: 82 - DNA, 20 bp, Artificial Sequence
  Description of Artificial Sequence Synthetic primer
tgaaagcaagcaaacaggtg

SEQ ID NO: 83 - DNA, 20 bp, Artificial Sequence
  Description of Artificial Sequence Synthetic primer
agcgctgtttttgttgtgtg

SEQ ID NO: 84 - DNA, 20 bp, Artificial Sequence
  Description of Artificial Sequence Synthetic primer
cttgcctcaaagtcctgctt

SEQ ID NO: 85 - DNA, 6291 bp, Artificial Sequence
  Description of Artificial Sequence Synthetic polynucleotide
atgtcgcggacccggctcccttccccacccgcacccagcccagcgttttcggccgactcgttctcagacctgcttaggcagttcgacccctcactgtttaacacatcgttgttcgactcccttcctccgtttggggcgcaccatacggaggcggccaccggggagtgggatgaggtgcagtcgggattgagagctgcggatgcaccacccccaaccatgcgggtggccgtcaccgctgcccgaccgccgagggcgaagcccgcaccaaggcggagggcagcgcaaccgtccgacgcaagccccgcagcgcaagtagatttgagaactttgggatattcacagcagcagcaggaaaagatcaagcccaaagtgaggtcgacagtcgcgcagcatcacgaagcgctggtgggtcatgggtttacacatgcccacatcgtagccttgtcgcagcaccctgcagcccttggcacggtcgccgtcaagtaccaggacatgattgcggcgttgccggaagccacacatgaggcgatcgtcggtgtggggaaacagtggagcggagcccgagcgcttgaggccctgttgacggtcgcgggagagctgagagggcctccccttcagctggacacgggccagttgctgaagatcgcgaagcggggaggagtcacggcggtcgaggcggtgcacgcgtggcgcaatgcgctcacgggagcacccctcaacctgaccccagagcaggtcgtggcaattgcgagccatgacgggggaaagcaggcactcgaaaccgtccagaggttgctgcctgtgctgtgccaagcgcacggacttacgccagagcaggtcgtggcaattgcgagccatgacgggggaaagcaggcactcgaaaccgtccagaggttgctgcctgtgctgtgccaagcgcacggactaaccccagagcaggtcgtggcaattgcgagcaacggagggggaaagcaggcactcgaaaccgtccagaggttgctgcctgtgctgtgccaagcgcacgggttgaccccagagcaggtcgtggcaattgcgagccatgacgggggaaagcaggcactcgaaaccgtccagaggttgctgcctgtgctgtgccaagcgcacggcctgaccccagagcaggtcgtggcaattgcgagccatgacgggggaaagcaggcactcgaaaccgtccagaggttgctgcctgtgctgtgccaagcgcacggactgacaccagagcaggtcgtggcaattgcgagcaacggagggggaaagcaggcactcgaaaccgtccagaggttgctgcctgtgctgtgccaagcgcacggacttacacccgaacaagtcgtggcaattgcgagcaacggagggggaaagcaggcactcgaaaccgtccagaggttgctgcctgtgctgtgccaagcgcacggacttacgccagagcaggtcgtggcaattgcgagccatgacgggggaaagcaggcactcgaaaccgtccagaggttgctgcctgtgctgtgccaagcgcacggactaaccccagagcaggtcgtggcaattgcgagccatgacgggggaaagcaggcactcgaaaccgtccagaggttgctgcctgtgctgtgccaagcgcacgggttgaccccagagcaggtcgtggcaattgcgagcaacggagggggaaagcaggcactcgaaaccgtccagaggttgctgcctgtgctgtgccaagcgcacggcctgaccccagagcaggtcgtggcaattgcgagcaacggagggggaaagcaggcactcgaaaccgtccagaggttgctgcctgtgctgtgccaagcgcacggactgacaccagagcaggtcgtggcaattgcgagcaaccacgggggaaagcaggcactcgaaaccgtccagaggttgctgcctgtgctgtgccaagcgcacggcctcaccccagagcaggtcgtggcaattgcgagccatgacgggggaaagcaggcactcgaaaccgtccagaggttgctgcctgtgctgtgccaagcgcacggacttacgccagagcaggtcgtggcaattgcgagccatgacgggggaaagcaggcactcgaaaccgtccagaggttgctgcctgtgctgtgccaagcgcacggactaaccccagagcaggtcgtggcaattgcgagcaacatcgggggaaagcaggcactcgaaaccgtccagaggttgctgcctgtgctgtgccaagcgcacgggttgaccccagagcaggtcgtggcaattgcgagcaacatcgggggaaagcaggcactcgaaaccgtccagaggttgctgcctgtgctgtgccaagcgcacggcctgaccccagagcaggtcgtggcaattgcgagccatgacgggggaaagcaggcactcgaaaccgtccagaggttgctgcctgtgctgtgccaagcgcacggactgacaccagagcaggtcgtggcaattgcgagcaaccacgggggaaagcaggcactcgaaaccgtccagaggttgctgcctgtgttatgtcaggcccatgggctcaccccagagcaggtcgtggcaattgcgagccatgacgggggaaagcaggcactcgaaaccgtccagaggttgctgcctgtgctgtgccaagcgcacggacttacgccagagcaggtcgtggcaattgcgagcaacggagggggaaagcaggcactcgaaaccgtccagaggttgctgcctgtgctgtgccaagcgcacggactaaccccagagcaggtcgtggcaattgcgagcaaccacgggggaaagcaggcactcgaaaccgtccagaggttgctgcctgtgctgtgccaagcgcacgggttgaccccagagcaggtcgtggcaattgcgagcaaccacgggggaaagcaggcactcgaaaccgtccagaggttgctgcctgtgctgtgccaagcgcacggcctgaccccagagcaggtcgtggcaattgcgagccatgacgggggaaagcaggcactcgaaaccgtccagaggttgctgcctgtgctgtgccaagcgcacggactgacaccagagcaggtcgtggcaattgcgagcaacggagggggaaagcaggcactcgaaaccgtccagaggttgctgcctgtgctgtgccaagcgcacggactcacgcctgagcaggtagtggctattgcatcccatgacgggggcagacccgcactggagtcaatcgtggcccagctttcgaggccggaccccgcgctggccgcactcactaatgatcatcttgtagcgctggcctgcctcggcggacgacccgccttggatgcggtgaagaaggggctcccgcacgcgcctgcattgattaagcggaccaacagaaggattcccgagaggacatcacatcgagtggcagatcacgcgcaagtggtccgcgtgctcggattcttccagtgtcactcccaccccgcacaagcgttcgatgacgccatgactcaatttggtatgtcgagacacggactgctgcagctctttcgtagagtcggtgtcacagaactcgaggcccgctcgggcacactgcctcccgcctcccagcggtgggacaggattctccaagcgagcggtatgaaacgcgcgaagccttcacctacgtcaactcagacacctgaccaggcgagccttcatgcgttcgcagactcgctggagagggatttggacgcgccctcgcccatgcatgaaggggaccaaactcgcgcgtcagcaagccccaagaagaagagaaaggtggaggccagcggtaccaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgagcagcggcaacagcaacgccaacagccgcggccccagcttcagcagcggcctggtgcccctgagcctgcgcggcagccacatgggccctatggagatatacaagacagtgtctgcatggaagagacagccagtgcgggtactgagcctttttagaaatattgataaagtactaaagagtttgggctttttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccgaatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttgctagcgactacaaagaccatgacggtgattataaagatcatgacatcgactacaaggatgacgatgacaagtctagggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgaatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 86 - DNA, 1836 bp, Artificial Sequence
  Description of Artificial Sequence Synthetic polynucleotide
ctgacccccgagcaagtcgtcgccatcgccagtaacggaggcggcaaacaagccctggagactgtccaacgacttttgcctgttctctgccaggcccatggtttgactcctgagcaggtggtggctatagcttcccatgacggtggtaaacaagctttggagactgttcagaggctgctgccggtgttgtgtcaagcgcacggccttacgccggaacaggtcgtggcgattgcgtcacatgacggggggaaacaagcgctcgaaaccgtgcaacggcttctgcccgtcttgtgccaggctcatgggctcaccccggagcaggttgttgctatcgcatctaacggaggaggcaagcaagcgttggaaacggttcaaaggcttctccccgtgctctgtcaagcccacggacttactccagagcaggtagtcgcgatagcgagtaacggagggggtaagcaagctctcgaaactgtacagagactgcttccggtactttgccaggcacatggcttgacaccggagcaagttgtagccattgcctcaaaccacggtgggaagcaagcccttgagacagtccagcgattgctgcctgtattgtgtcaagctcacggtttgacgcccgaacaagtcgtagctatcgctagccatgacggcgggaagcaggctcttgaaacagttcagcggttgcttcccgtactctgtcaggctcacggcctgactcccgaacaggtcgttgccatagcctctcatgacggcggtaaacaggccttggaaactgtccaaagacttcttcctgtcttgtgtcaggcccacgggcttacccccgagcaggttgtggccattgcatcaaacatcggaggtaagcaggcgctggagactgtgcaacgccttctgccagttttgtgccaggcgcacggacttacacctgaacaggtagtggcaattgcaagcaacatcggagggaaacaggccctcgaaacggtacagcggctgctccctgtgctctgccaagcccatggattgaccccagagcaagtagttgcgatagcctcacatgacggtggaaagcaggcattggagacagtgcagcgcctgttgcccgttctttgccaagctcatggtcttacacccgagcaagtggtcgcaatcgcgtctaaccacgggggcaaacaggctctcgaaaccgtacaacgactccttccagtgctttgtcaagcgcatggcctcacgccagaacaggtggtagcgatcgcgagccatgacggggggaaacaggcacttgaaaccgttcaacgcctcttgccagtcctgtgccaggcacacggtctgacaccagagcaggtggttgcaattgcgagtaacggaggagggaagcaagcactcgaaacagtacagcgactcctccctgtgctgtgccaagctcacggattgacccctgaacaagttgtagcgatagcatctaaccacggcggaaaacaggcgctggaaacggtgcaaaggctccttcccgttctttgtcaggcccatggcctcactccggaacaagtcgtcgctattgctagtaaccacggtggcaagcaggcccttgaaacggtccagaggttgttgccggtcctctgtcaagcacatgggctcacaccagaacaagtagtagcaatagccagccatgacggaggaaaacaagcacttgagacagtacaaagactcctcccagtactgtgccaagcacacgggctgacgcctgaacaagtggttgcaatcgcttcaaacggaggtggaaagcaagctctggaaactgttcagcgcttgctcccggttctgtgtcaggcgcatgga